(19) United States
(12) Patent Application Publication
(10) Pub. No.: US 2026/0258295 A1
(43) Pub. Date: Sep. 3, 2026

(54) AZA[3.2.1]BRIDGED RING SUBSTITUTED FLUORESCENT DYE AND PREPARATION METHOD AND USE THEREOF

(71) Applicants: PEKING UNIVERSITY, Beijing (CN); NANJING GENVIVO BIOTECH CO., LTD., Jiangsu (CN)

(72) Inventors: Zhixing Chen, Beijing (CN); Junwei Zhang, Beijing (CN); Mingqiao Liu, Beijing (CN); Peng Chen, Beijing (CN); Jingfu Sun, Beijing (CN)

(21) Appl. No.: 18/726,470

(22) PCT Filed: Sep. 6, 2023
(86) PCT No.: PCT/CN2023/117328

§ 371 Date: Jul. 3, 2024

(30) Foreign Application Priority Data
Jul. 3, 2023 (CN) 202310809062.6

(51) Int. Cl. C09K 11/06; C09B 11/02; C09B 11/28; G01N 21/64; G01N 33/58

(52) U.S. Cl. CPC C09K 11/06; C09B 11/02; C09B 11/28; G01N 21/6428; G01N 33/582; G01N 2021/6439

(57) ABSTRACT

The present disclosure provides an aza[3.2.1]bridged ring substituted fluorescent dye and a preparation method and use thereof. According to the fluorescent dye, by adopting an aza[3.2.1]bridged ring as a substituent of the fluorescent dye, not only are the brightness and photostability of the fluorescent dye obviously improved, but also the phenomenon of spectral blue shift caused by irradiation of the dye for a long time is completely avoided. Meanwhile, the water solubility of the fluorescent dye can be effectively improved, and good membrane permeability of the fluorescent dye is maintained. The present disclosure further provides embodiments of functionalized derivatives of the dye in single molecule imaging, confocal imaging and super-resolution imaging of biological samples.

### CROSS-REFERENCE TO RELATED APPLICATION

The present disclosure is a U.S. national phase patent application of PCT/CN2023/117328 filed Sep. 6, 2023 which claims the priority of a Chinese patent application No. 2023108090626, filed to China National Intellectual Property Administration on Jul. 3, 2023, with the title of “Aza[3.2.1]bridged Ring Substituted Fluorescent Dye and Preparation Method and Use thereof”, the entire contents of which are incorporated herein by reference.

### TECHNICAL FIELD

The present disclosure relates to the field of fluorescent dyes in biofluorescence analysis, and specifically relates to an aza[3.2.1]bridged ring substituted fluorescent dye and a preparation method and use thereof.

### BACKGROUND

In recent years, fluorescent dyes have been widely used in the fields of biological macromolecular labeling, ion detection, single molecule imaging, super-resolution imaging and the like, and have become an irreplaceable important tool in life science research. Thus, higher requirements on physical and chemical properties of the fluorescent dyes have been put forward. Traditional fluorescent dyes, such as fluorescein, tetramethylrhodamine and coumarin, are usually in lack of sufficient brightness, water solubility and photostability. In the past three decades, different novel substituents have been introduced into matrices of the fluorescent dyes by chemists to improve the fluorescence quantum yield, photostability, resistance to photooxidation dealkylation (spectral blue shift), water solubility and membrane permeability of the dyes. However, the challenge is that currently reported strategies for optimizing the fluorescent dyes can be used for only optimizing a few properties separately and cannot be used for comprehensively improving all the core properties of the dyes.

According to the document Nature Methods. 2015, 12:244-250, the brightness of a fluorescent dye is systematically improved by an azacyclobutane substitution method, but the dye has poor photostability and no resistance to photooxidation dealkylation (spectral blue shift). According to the document J. Am. Chem. Soc. 2008, 130, 52:17652-17653, the brightness, photostability and resistance to photooxidation dealkylation (spectral blue shift) of a dye are improved by a method of substituting rhodamine with azabicyclo[2.2.1]heptane, but the dye has poor water solubility and membrane permeability, so that use of the dye in living cell imaging is hindered. By introducing a sulfonic acid group into a matrix of a fluorescent dye, the water solubility of the dye can be greatly improved, the antibody labeling efficiency is improved, and non-specific dyeing is reduced, but the dye is also prevented from permeating a cell membrane, so that the sulfonated dye cannot be used in living cells.

Therefore, it is urgent for persons skilled in the art to develop a class of fluorescent dyes capable of simultaneously improving the fluorescence quantum yield, the photostability, the resistance to photooxidation dealkylation (spectral blue shift), the water solubility and the membrane permeability.

### SUMMARY

The main purpose of the present disclosure is to provide an aza[3.2.1]bridged ring substituted fluorescent dye and a preparation method and use thereof, so as to solve the problems that the fluorescence quantum yield, photostability, resistance to photooxidation dealkylation (spectral blue shift), water solubility and membrane permeability of fluorescent dyes in the prior art cannot be optimized simultaneously.

In order to achieve the above purpose, an aza[3.2.1]bridged ring substituted fluorescent dye is provided according to one aspect of the present disclosure. The aza[3.2.1]bridged ring substituted fluorescent dye has a structure shown in Formula (I), Formula (II) or Formula (III) below:

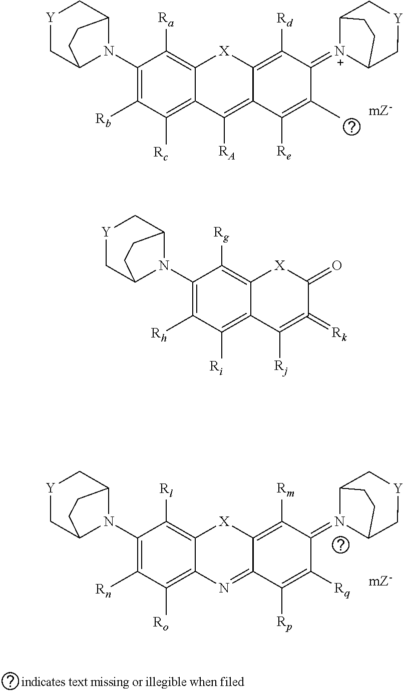

Rₐ, R_{b}, R_{c}, R_{d}, Rₑ, R_{f}, R_{g}, Rₕ, Rᵢ, Rⱼ, Rₖ, Rₗ, Rₘ, Rₙ, Rₒ, Rₚ and R_{q} are independently selected from hydrogen, halogen, carboxyl, substituted or unsubstituted C₁-C₂₀ alkyl, substituted or unsubstituted C₃-C₂₀ cycloalkyl, substituted or unsubstituted C₄-C₂₀ cycloalkylalkyl, C₄-C₂₀ alkylcycloalkyl, substituted or unsubstituted C₁-C₂₀ alkoxyl, substituted or unsubstituted C₁-C₂₀ ester group, and substituted or unsubstituted C₁-C₂₀ acylamino;
R_{A} is selected from substituted or unsubstituted C₆-C₄₀ aryl, substituted or unsubstituted C₆-C₄₀ aralkyl, and substituted or unsubstituted C₅-C₄₀ heteroaryl;
X is selected from oxygen, sulfur, substituted or unsubstituted C₁-C₂₀ alkylene,

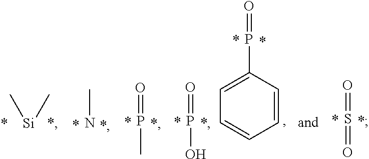

Y is selected from oxygen, sulfur, nitrogen, substituted or unsubstituted C₁-C₂₀ alkylene, substituted or unsubstituted C₁-C₂₀ alkylenoxyl, substituted or unsubstituted C₁-C₂₀ acyl, substituted or unsubstituted C₁-C₂₀ ester group,

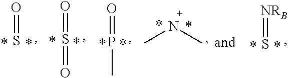

where R_{B} is selected from substituted or unsubstituted C₁-C₂₀ alkyl, substituted or unsubstituted C₁-C₂₀ alkoxyl, substituted or unsubstituted C₁-C₂₀ acyl, and substituted or unsubstituted C₁-C₂₀ ester group;
Z⁻ is selected from F⁻, Cl⁻, Br⁻, I⁻, OAc⁻, HSO₄⁻, H₂PO₄⁻, ClO₄⁻, CF₃COO⁻, CF₃SO₃⁻, CH₃SO₃⁻, NO₃⁻, C₅H₇O₅COO⁻, and PF₆⁻; and m is an integer ranged from 1 to 3.

Further, the Rₐ, the R_{b}, the R_{c}, the Rₐ, the Rₑ, the R_{f}, the R_{g}, the Rₕ, the Rᵢ, the Rⱼ, the Rₖ, the Rₗ, the Rₘ, the Rₙ, the Rₒ, the Rₚ and the R_{q} are independently selected from hydrogen, halogen, carboxyl, substituted or unsubstituted C₁-C₁₀ alkyl, substituted or unsubstituted C₃-C₁₀ cycloalkyl, substituted or unsubstituted C₄-C₁₀ cycloalkylalkyl, C₄-C₁₀ alkylcycloalkyl, substituted or unsubstituted C₁-C₁₀ alkoxyl, substituted or unsubstituted C₁-C₁₀ ester group, and substituted or unsubstituted C₁-C₁₀ acylamino; and/or, the R_{A} is selected from substituted or unsubstituted C₆-C₃₀ aryl, substituted or unsubstituted C₆-C₃₀ aralkyl, and substituted or unsubstituted C₅-C₃₀ heteroaryl; and/or, the X is selected from oxygen, sulfur, substituted or unsubstituted C₁-C₁₀ alkyl,

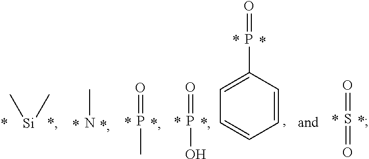

and/or, the Y is selected from oxygen, sulfur, nitrogen, substituted or unsubstituted C₁-C₁₀ alkyl, substituted or unsubstituted C₁-C₁₀ alkoxyl, substituted or unsubstituted C₁-C₁₀ acyl, substituted or unsubstituted C₁-C₁₀ ester group,

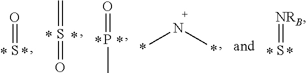

where the R_{B} is selected from substituted or unsubstituted C₁-C₂₀ alkyl, substituted or unsubstituted C₁-C₂₀ alkoxyl, substituted or unsubstituted C₁-C₂₀ acyl, and substituted or unsubstituted C₁-C₂₀ ester group; and/or, the Z is selected from F⁻, Cl⁻, Br⁻, I⁻, OAc⁻, HSO₄⁻, H₂PO₄⁻, ClO₄⁻, CF₃COO⁻, CF₃SO₃⁻, CH₃SO₃⁻, NO₃⁻, C₅H₇O₅COO⁻, and PF₆⁻; and/or, the m is an integer ranged from 1 to 3.

Further, the aza[3.2.1]bridged ring substituted fluorescent dye shown in Formula (I) has a structure shown in Formula (IV) below:

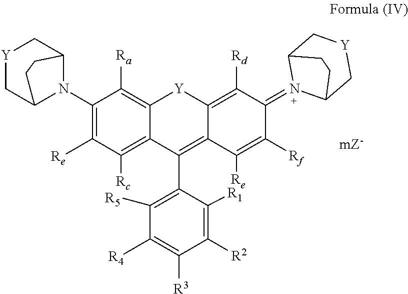

R₁ and R₅ are independently selected from hydrogen, carboxyl, halogen, substituted or unsubstituted C₁-C₆ alkyl, substituted or unsubstituted C₁-C₆ alkoxyl, nitro, amino, hydroxyl,

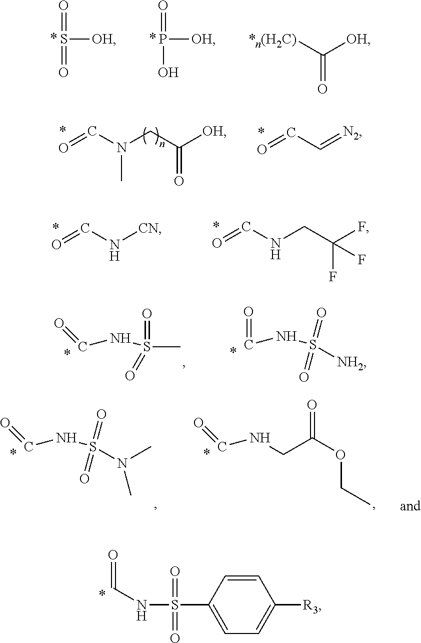

where R_{D} is selected from hydrogen, halogen, cyano, substituted or unsubstituted C₁-C₆ alkyl, and substituted or unsubstituted C₁-C₆ alkoxyl, and n is an integer ranged from 1 to 6; and R₂, R₃ and R₄ are independently selected from hydrogen, halogen, carboxyl, substituted or unsubstituted C₁-C₁₀ alkyl, substituted or unsubstituted C₃-C₁₀ cycloalkyl, substituted or unsubstituted C₄-C₁₀ cycloalkylalkyl, C₄-C₁₀ alkylcycloalkyl, substituted or unsubstituted C₁-C₁₀ alkoxyl, substituted or unsubstituted C₁-C₁₀ ester group,

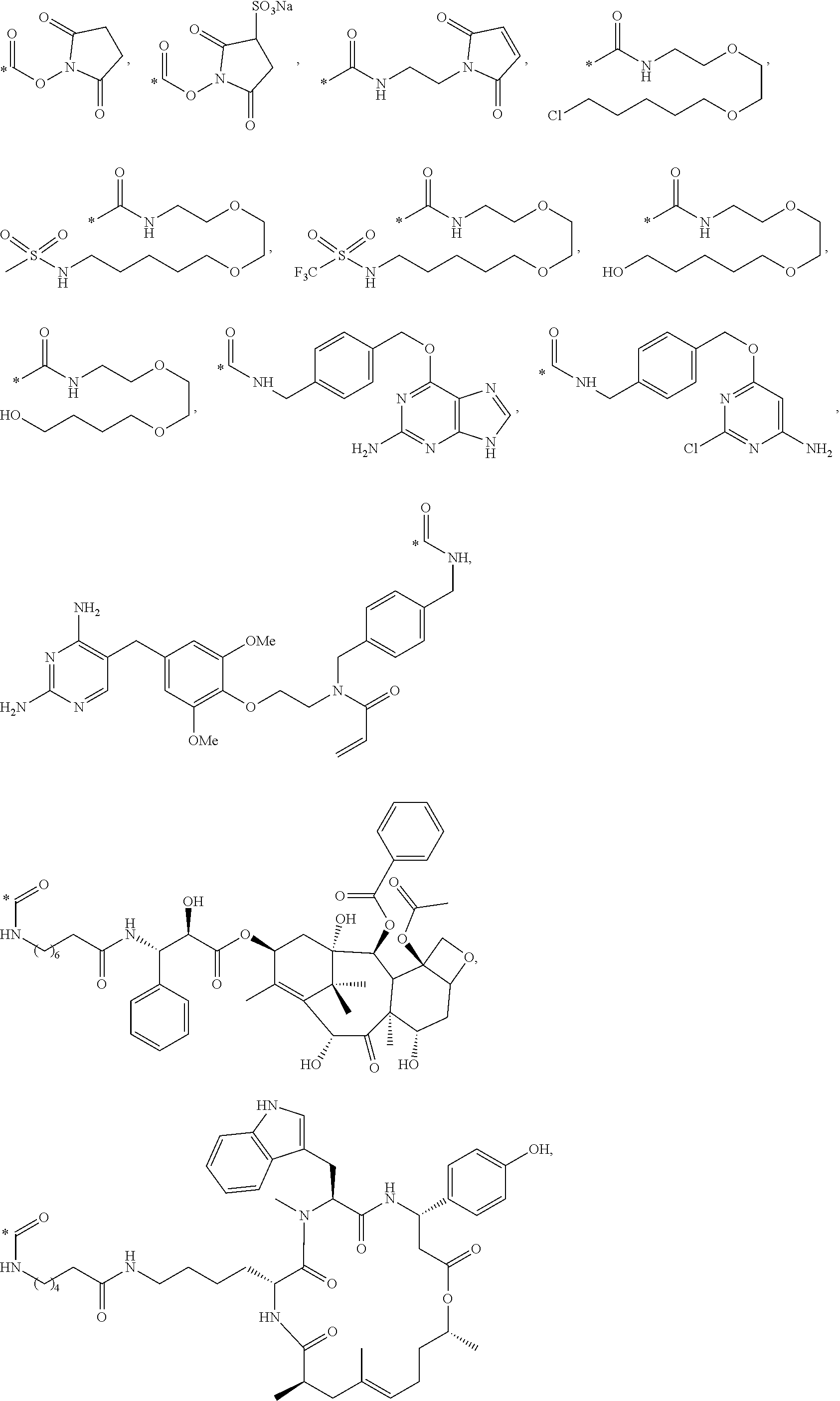

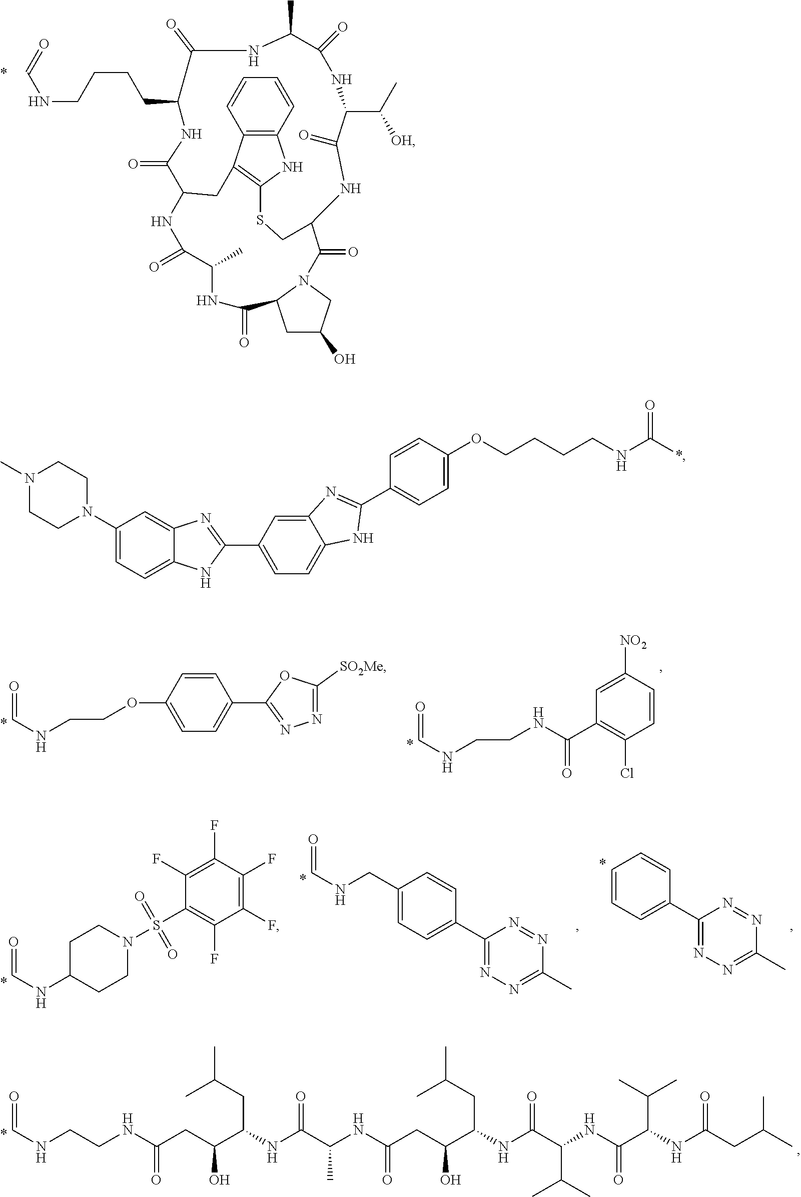

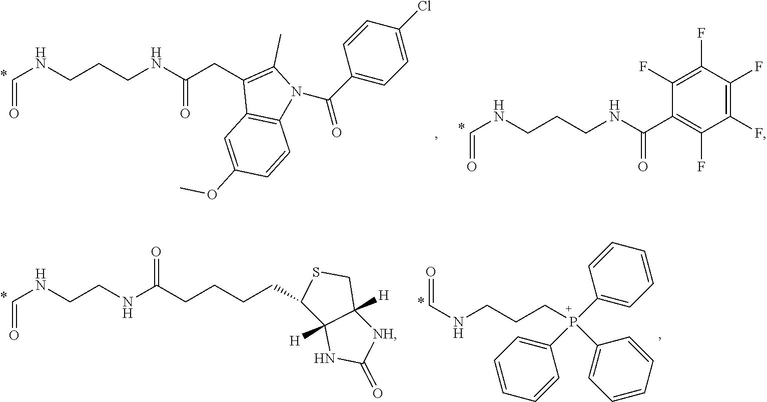

or any other functional groups used for biological coupling or subcellular localization.

Further, the aza[3.2.1]bridged ring substituted fluorescent dye is selected from at least one of the following formulas IA1-IA3, IIA1-IIA3, IIIA1-IIIA3, IB1-IB3, IC1-IC3, ID1-ID3, IE1-IE3, IF1-IF3, and IG1-IG3:

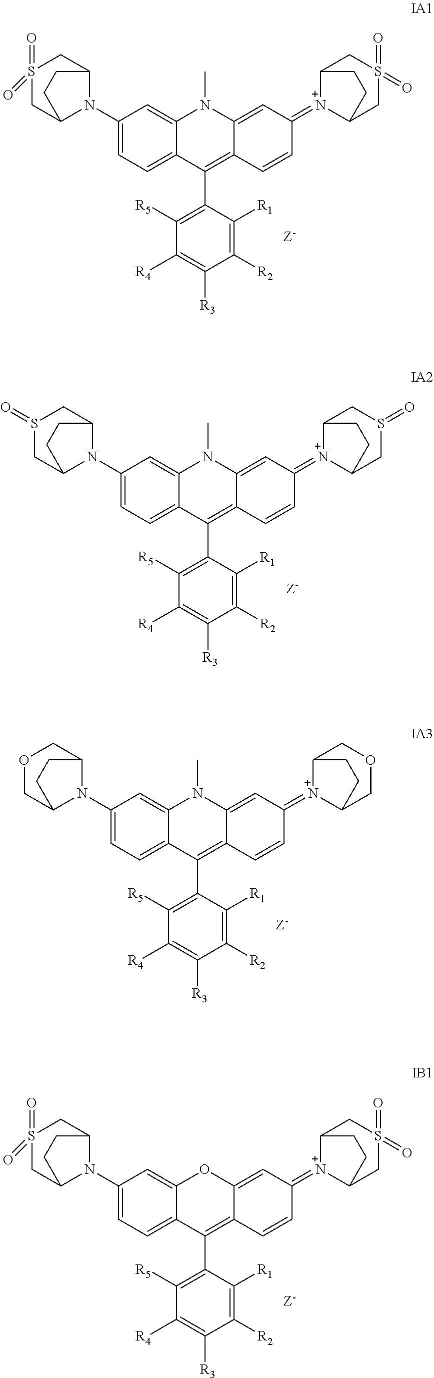

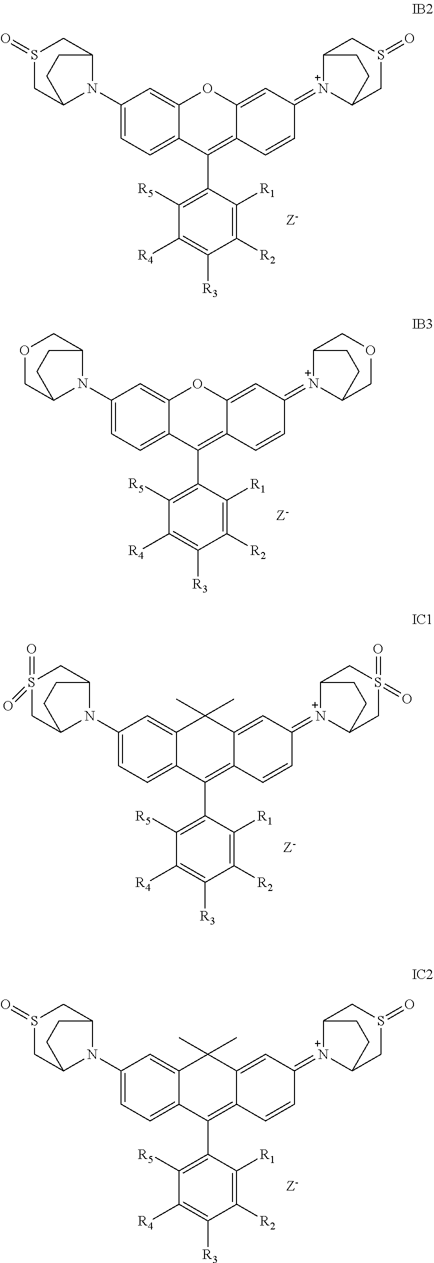

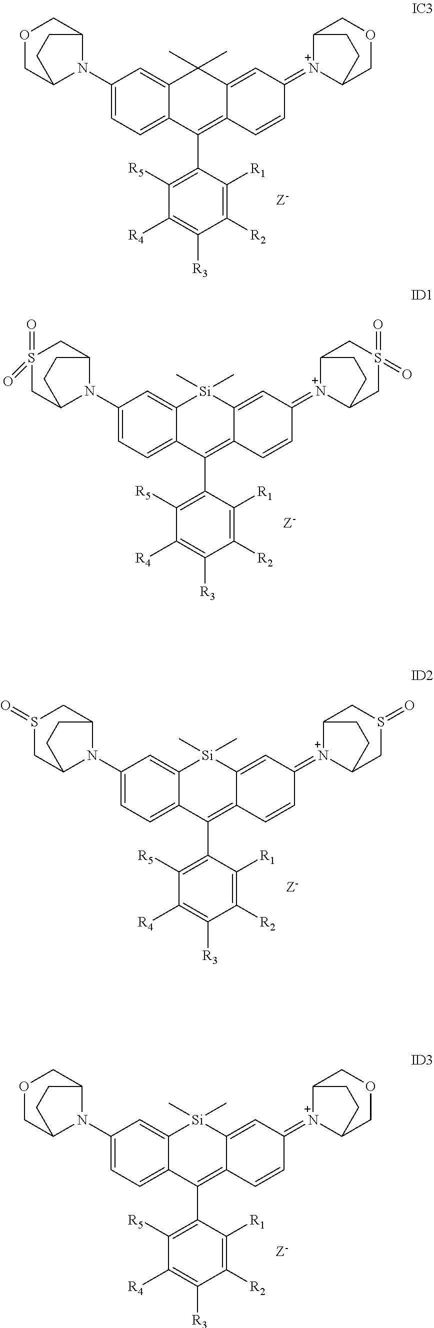

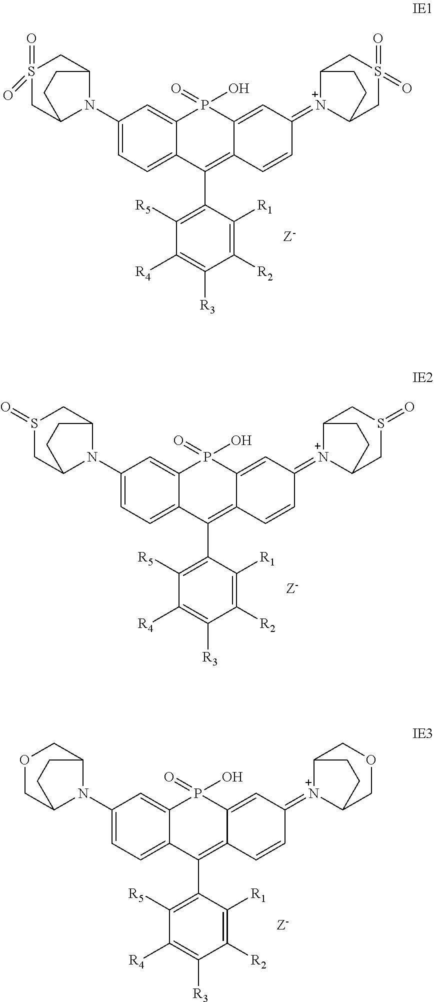

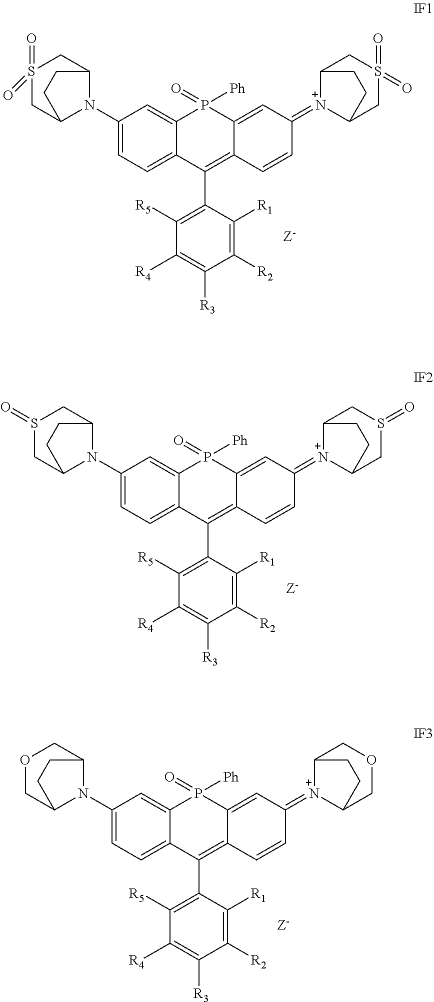

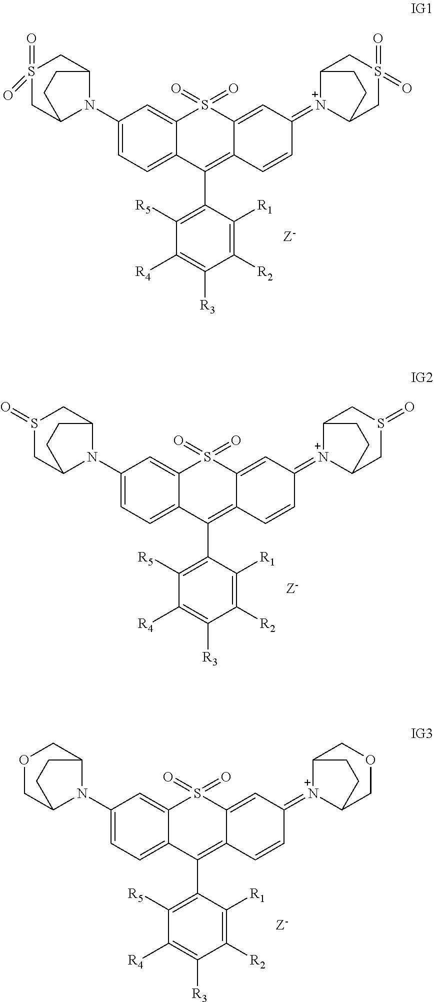

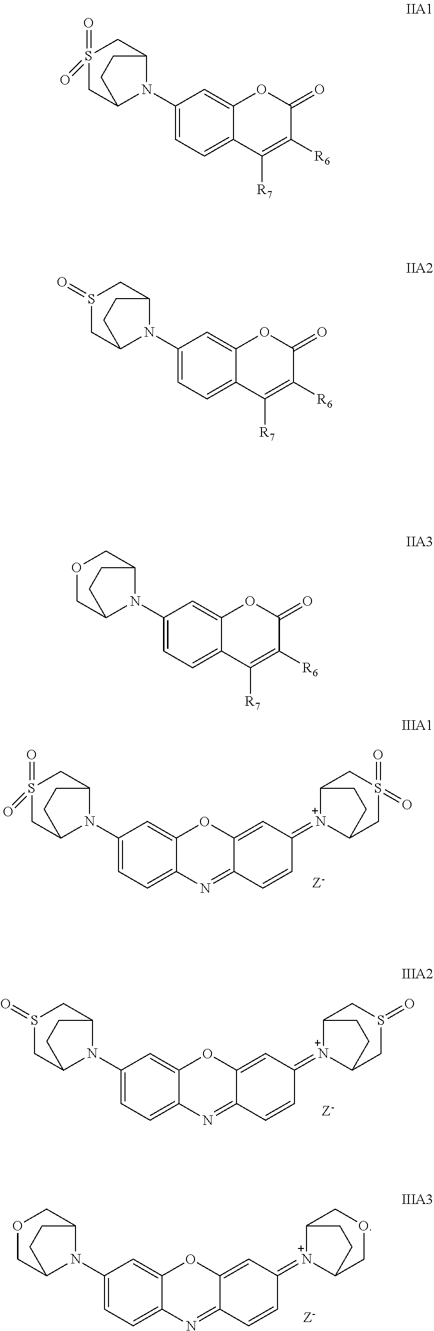

Further, the aza[3.2.1]bridged ring substituted fluorescent dye is selected from at least one of the following formulas A528, A532, A555, A586, A591, A620, A634, A640, A655, A720, A586-2Me, A586-2Me-5-COOH), A528-6-COOH), A532-6-COOH), A555-6-COOH), A586-6-COOH), A591-6-COOH), A620-6-COOH), A634-5-COOH), A640-6-COOH), A655-6-COOH), A528-HTL, A532-HTL, A555-HTL, A586-HTL, A620-HTL, A655-HTL, A555-STL, A528-NHS, A620-STL, A655-STL, A634-NHS, A586-NHS, A591-HTL, A640-HTL, A555-TMP3, A620-TMP3, A655-TMP3, A655-4-Hoechst, A591-TMP3, A640-TMP3, A591-STL, A640-STL, A528-NCN-HTL, A555-NCN-HTL, A620-NCN-HTL, B350, B356, C622, and C654, and Me in the following formulas represents methyl:

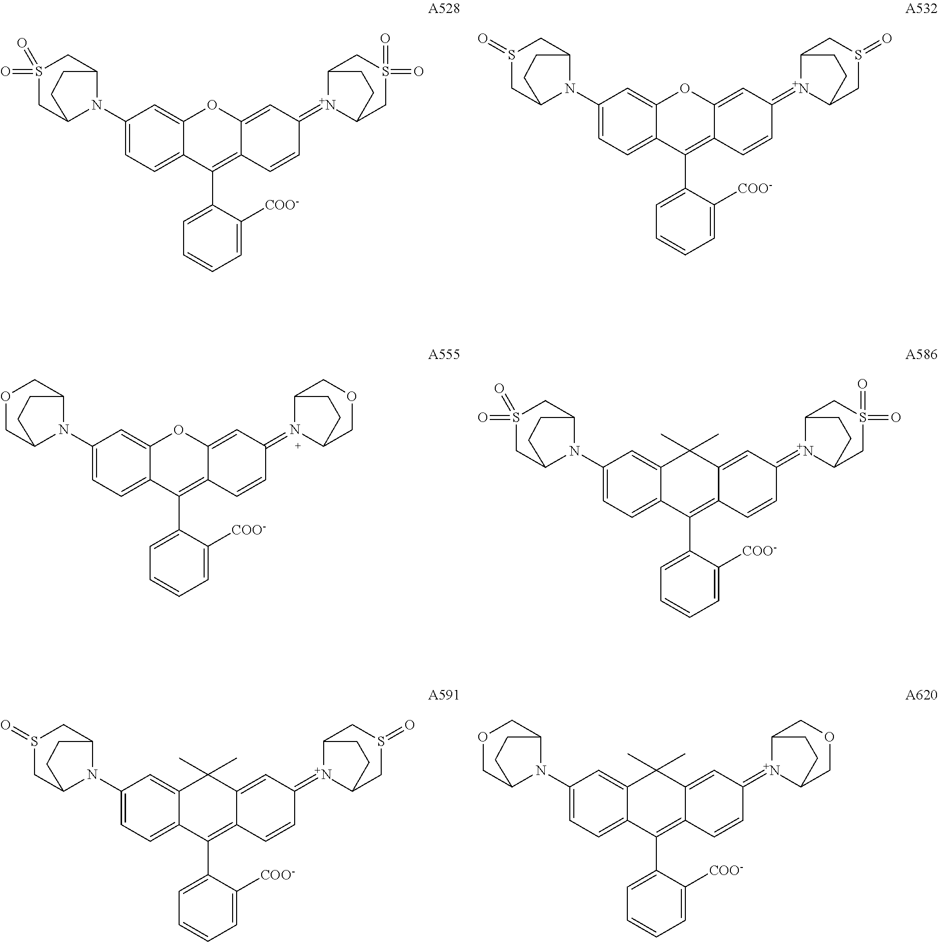

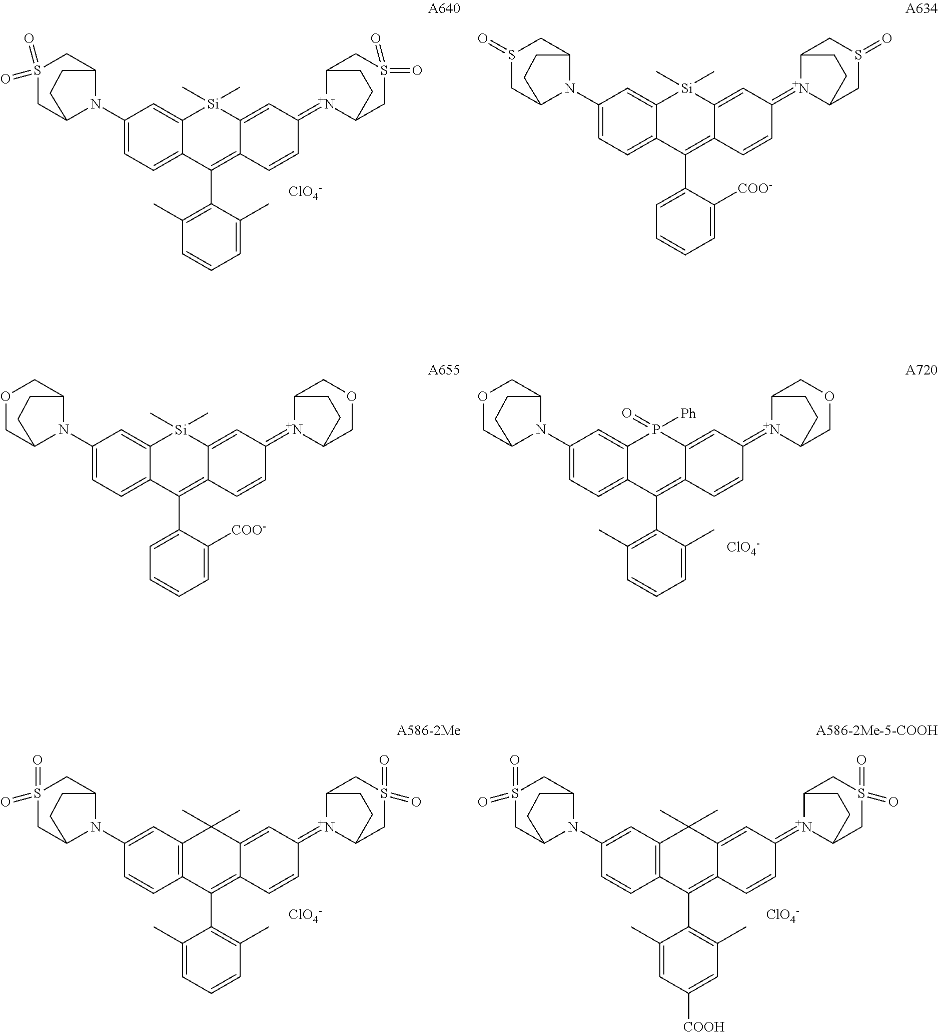

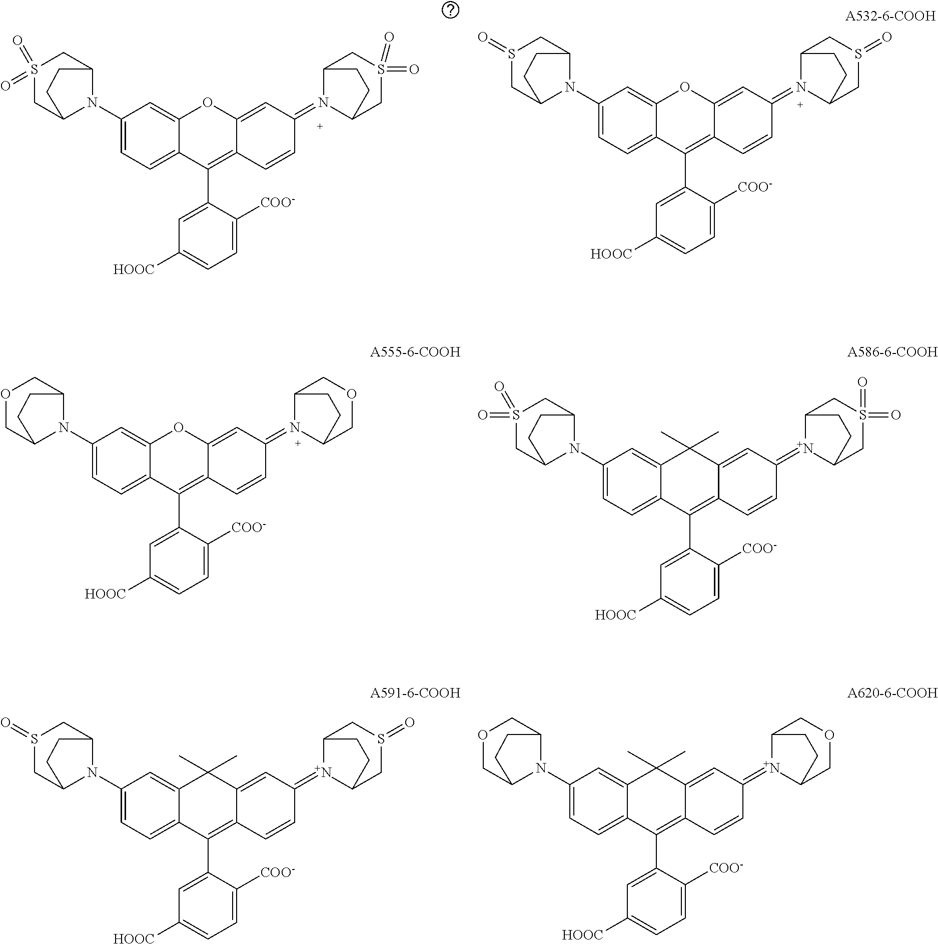

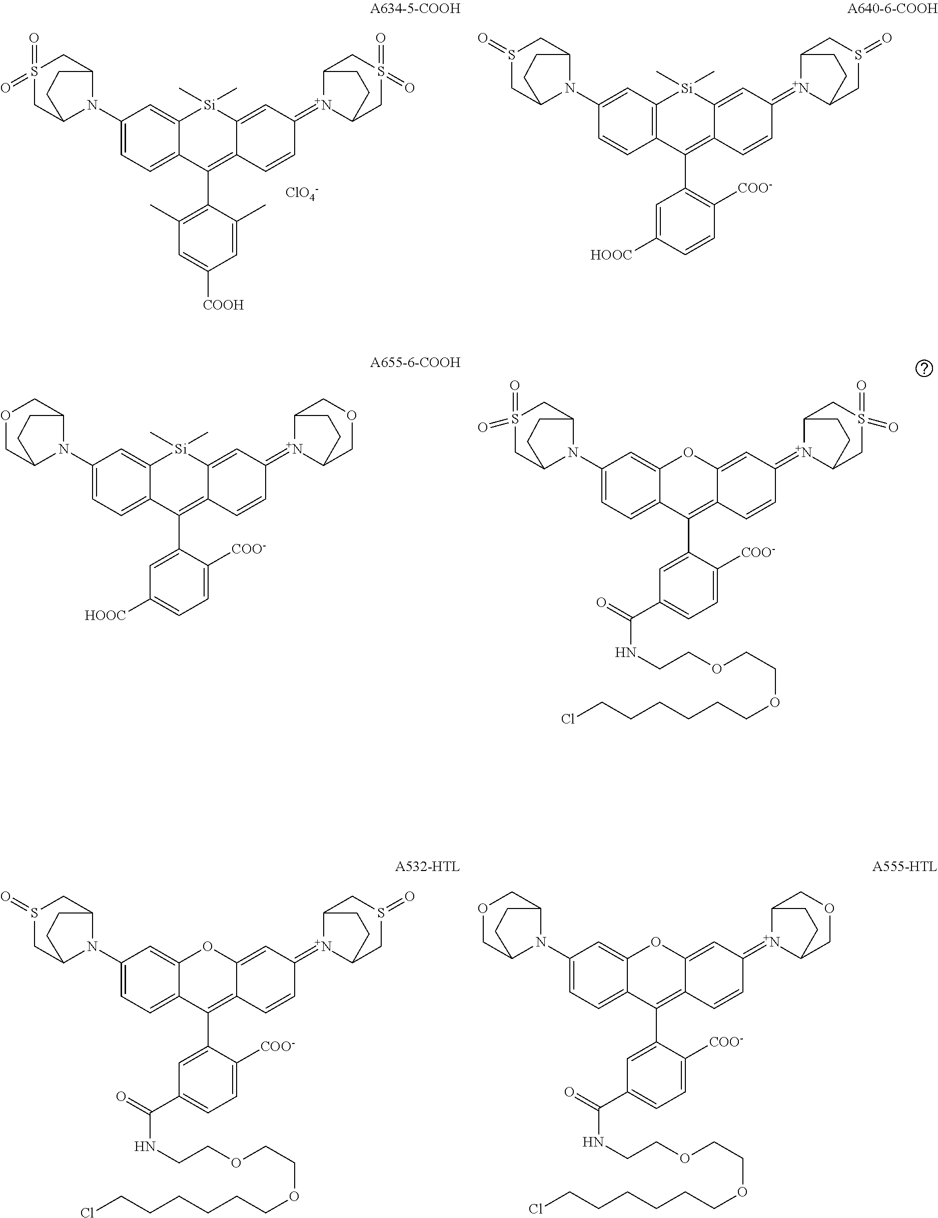

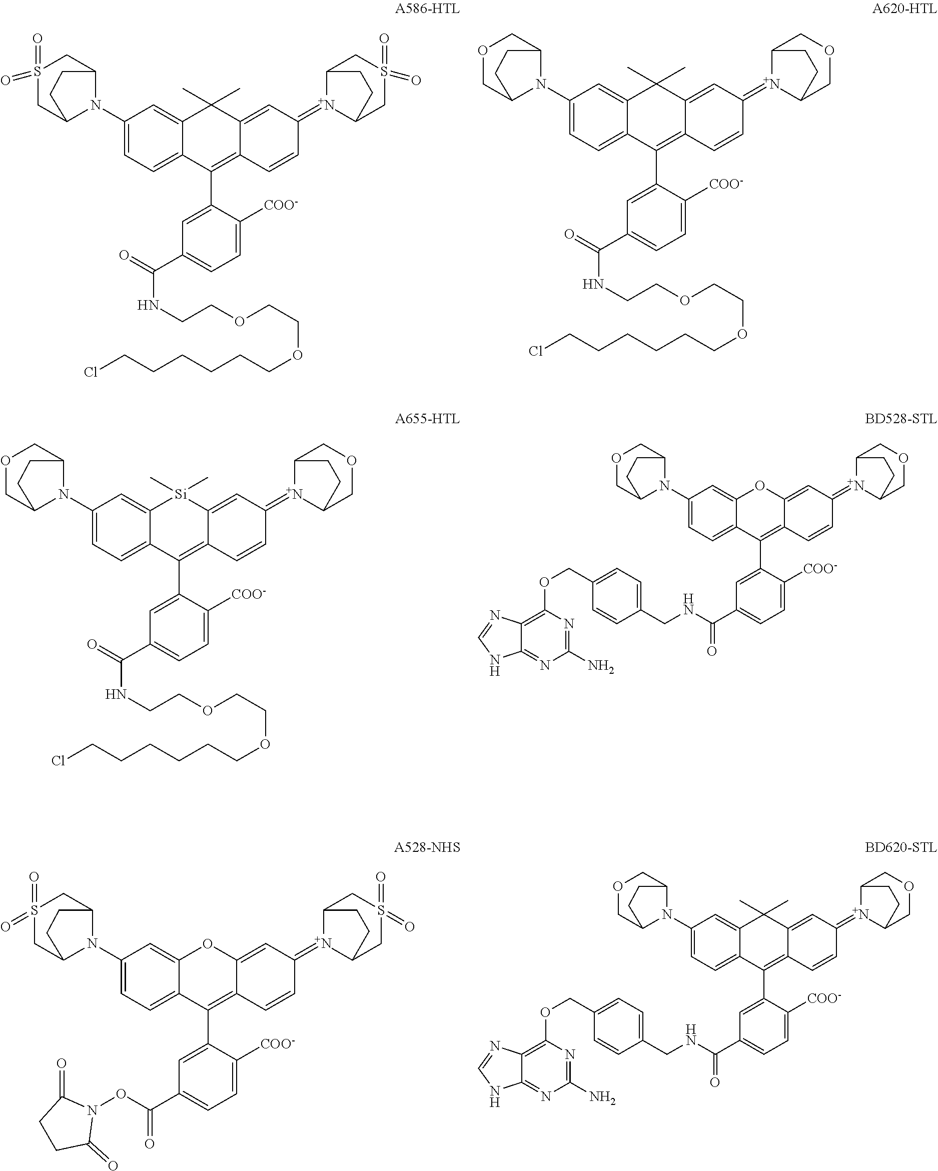

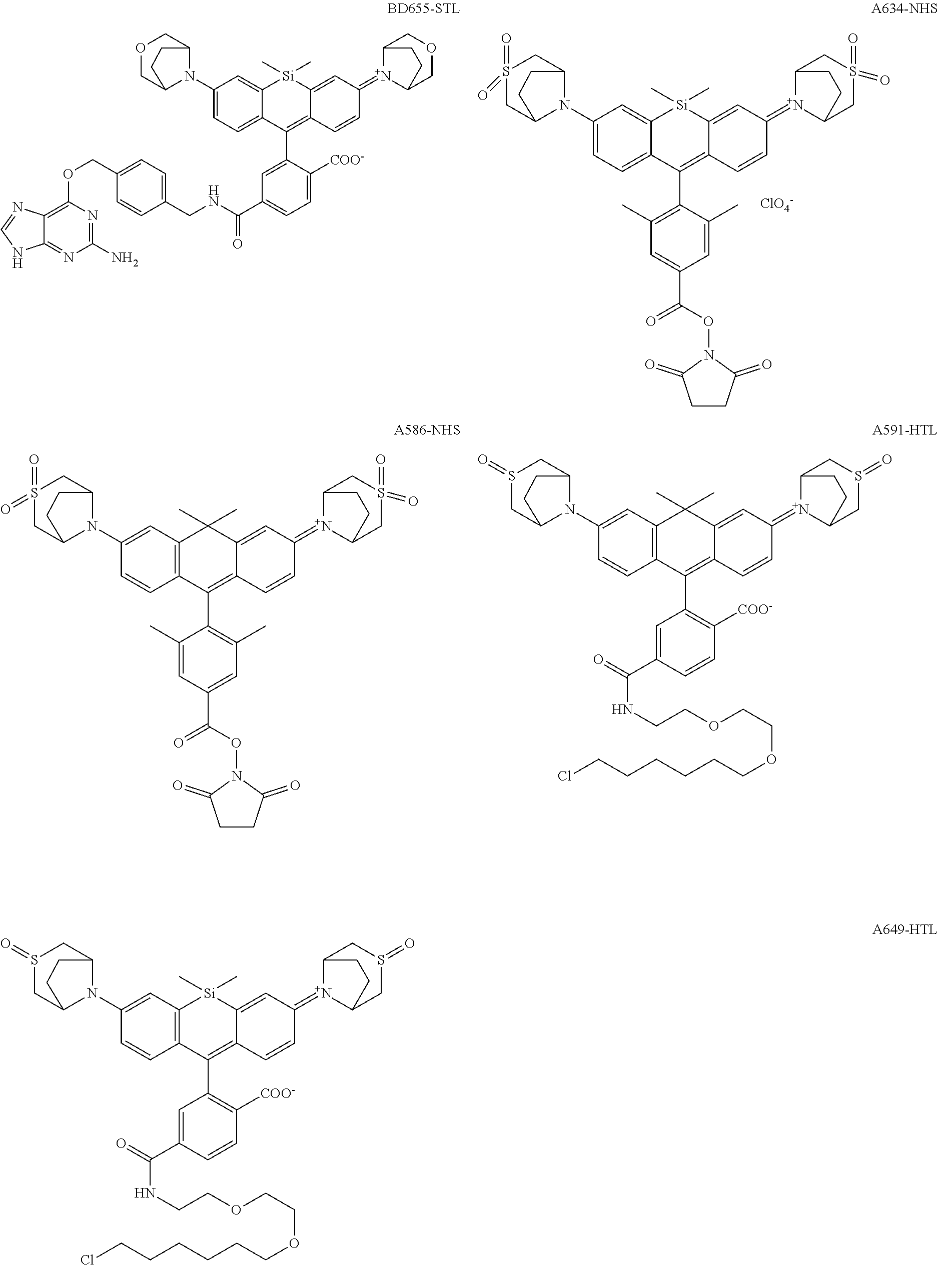

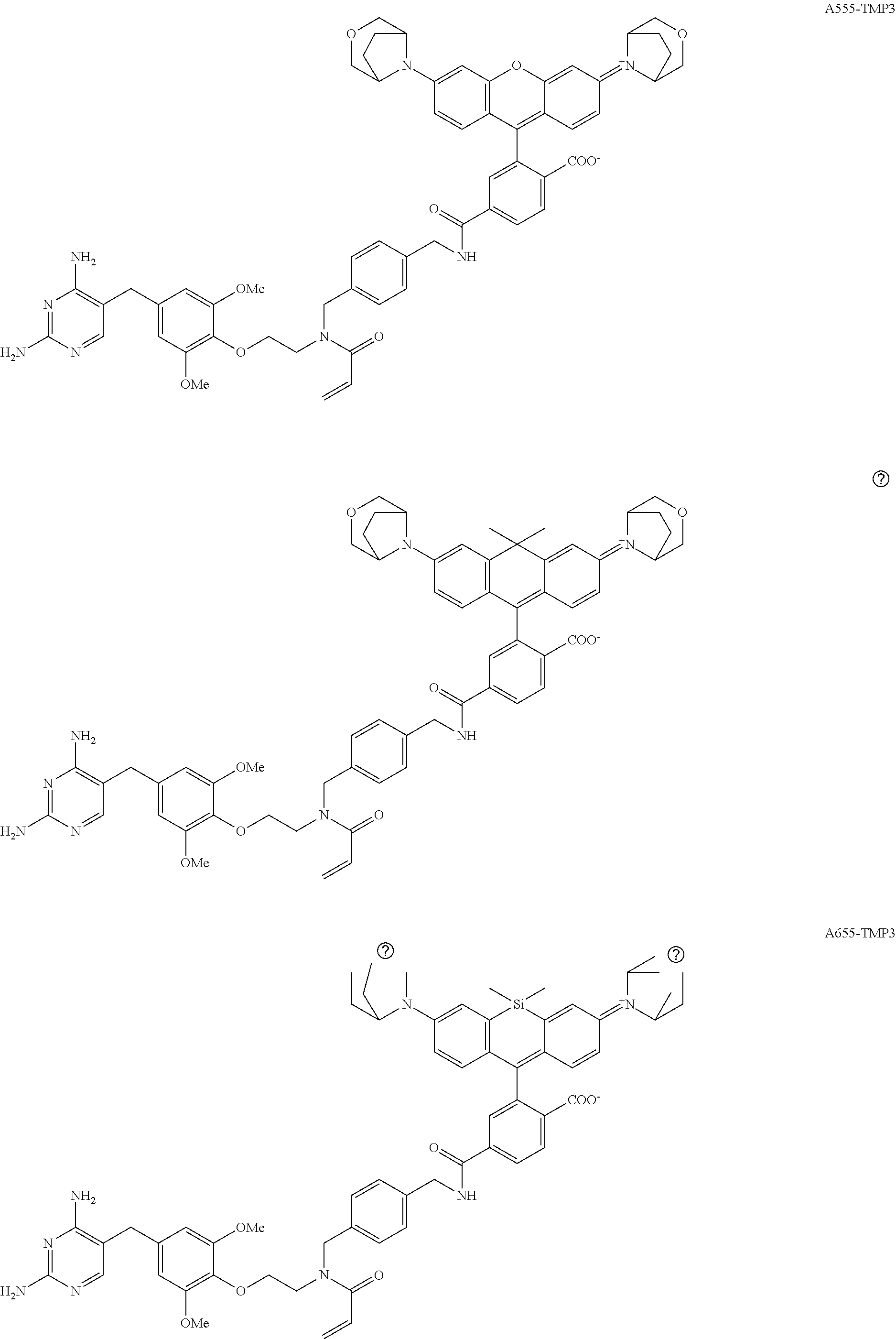

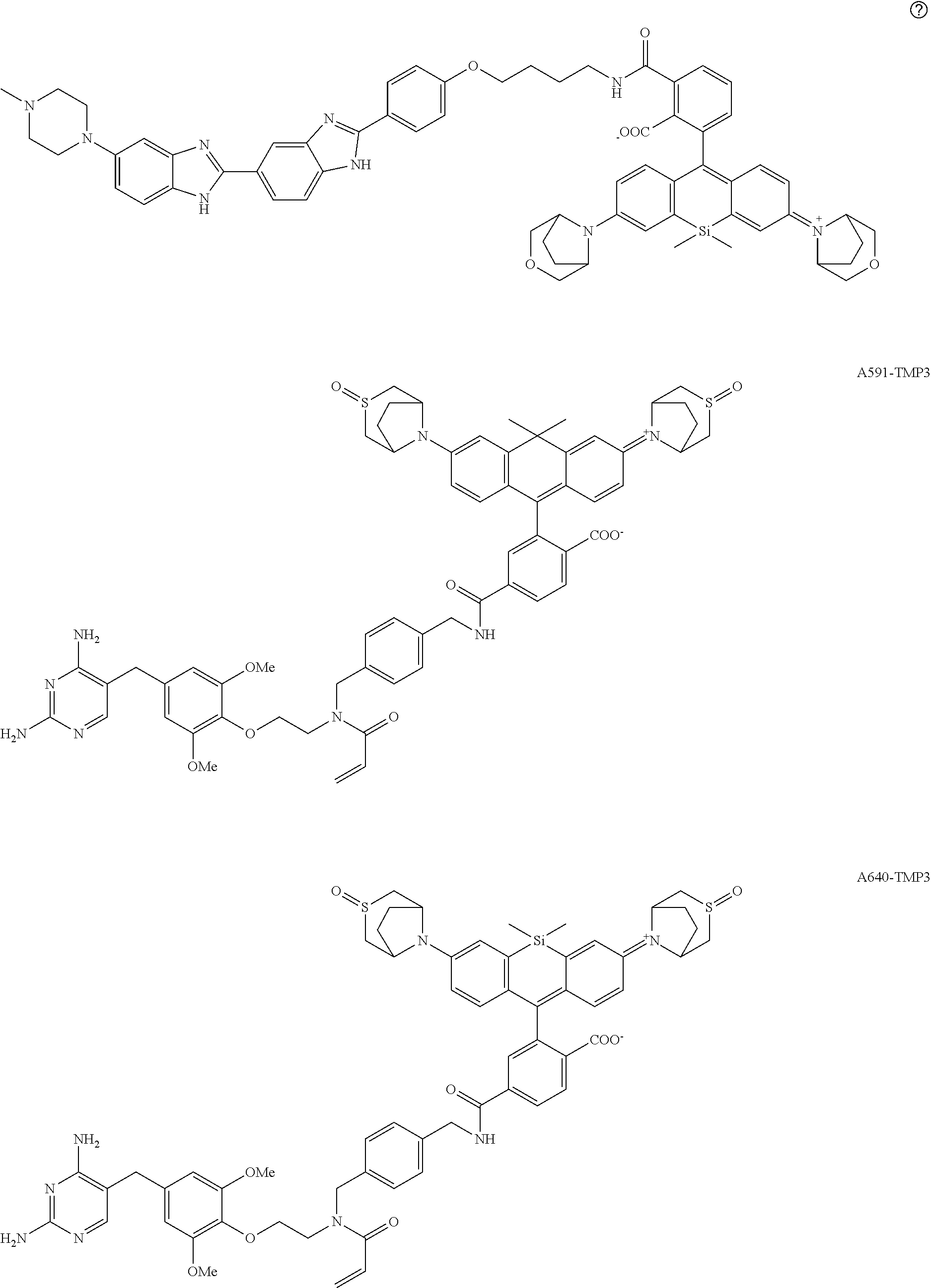

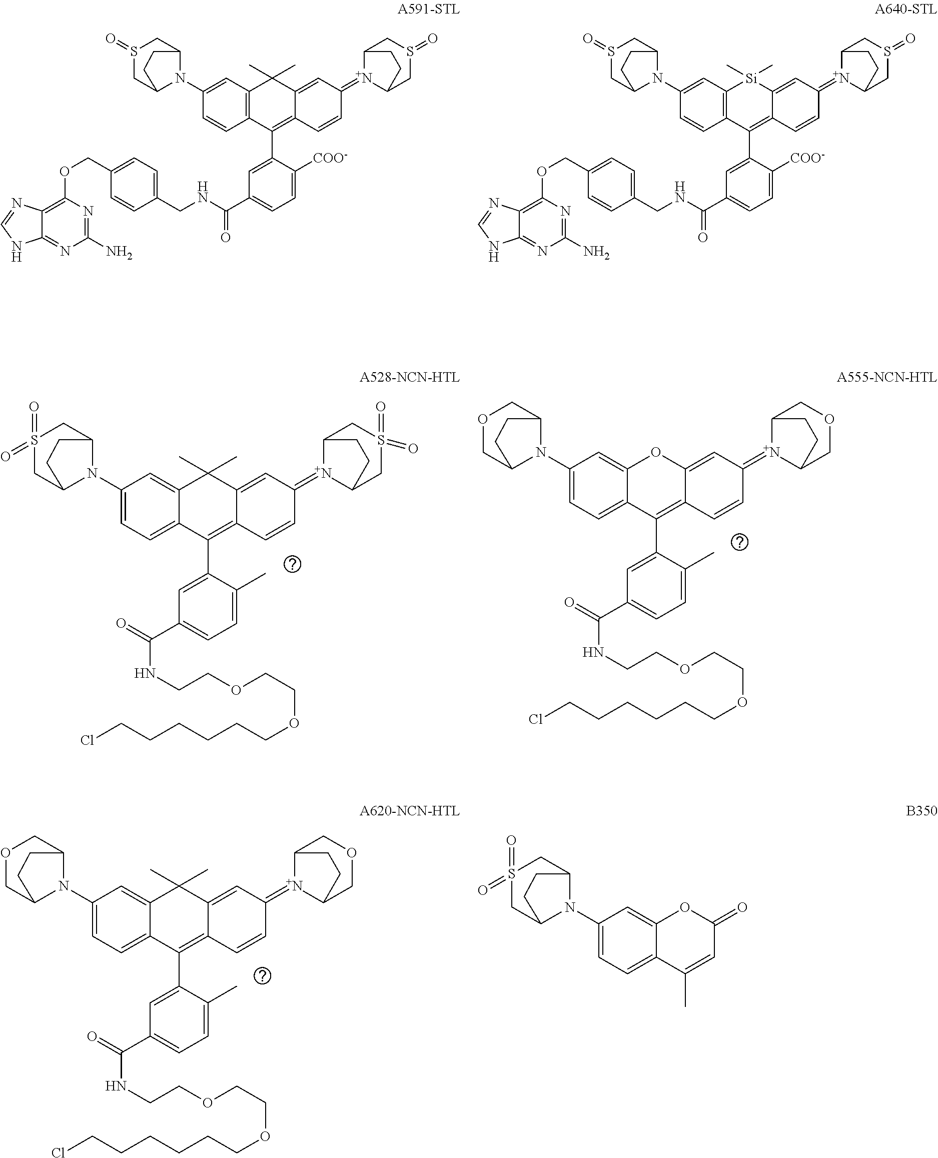

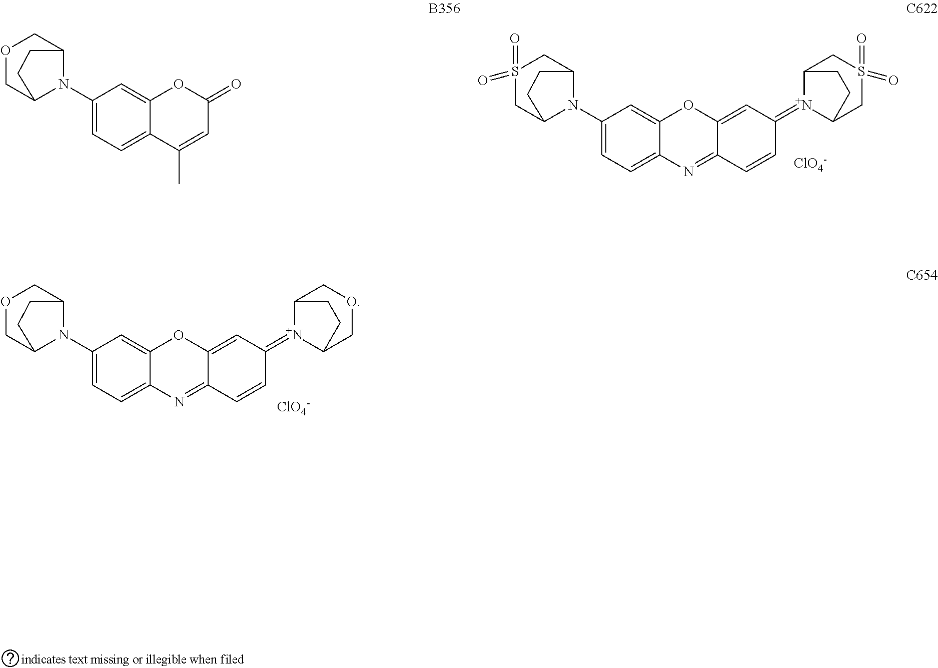

In order to achieve the above purpose, a method for preparing an aza[3.2.1]bridged ring substituted fluorescent dye is provided according to one aspect of the present disclosure. The preparation method includes: step S11, mixing a compound A, an aza[3.2.1]bridged ring compound and a first catalyst in a first reaction solvent to carry out a reaction so as to obtain a compound B; and step S12, mixing the compound B with an HZ aqueous solution to carry out a reaction so as to obtain the aza[3.2.1]bridged ring substituted fluorescent dye shown in Formula (I). The compound A has a structure shown in Formula (V) below, and the aza[3.2.1]bridged ring compound has a structure shown in Formula (VI) below:

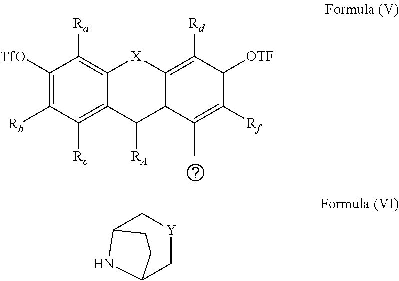

where R_{A}, Rₐ, R_{b}, R_{c}, Rₐ, Rₑ, R_{f}, X and Y independently have the same meaning as defined in the first aspect, Z⁻ in the HZ aqueous solution has the same meaning as defined in the first aspect, and OTf represents a trifluoromethanesulfonate group.

Further, the first catalyst includes a palladium catalyst, a palladium catalyst ligand and an alkaline catalyst.

Further, the palladium catalyst includes at least one of tetrakis(triphenylphosphine)palladium, palladium acetate, tris(dibenzylideneacetone)dipalladium, [(diphenylphosphino)ferrocene]dichloropalladium, dichlorobis(triphenylphosphine)palladium and palladium chloride.

Further, the palladium catalyst includes at least one of tetrakis(triphenylphosphine)palladium, palladium acetate or tris(dibenzylideneacetone)dipalladium.

Further, the palladium catalyst ligand includes at least one of bis(dibenzylideneacetone)palladium, 2,2′-bis(diphenylphosphino)-1,1′-binaphthyl, 2-dicyclohexylphosphino-2′,4′,6′-triisopropylbiphenyl and 2-dicyclohexylphosphino-2′,6′-dimethoxybiphenyl.

Further, the palladium catalyst ligand includes at least one of bis(dibenzylideneacetone)palladium and 2,2′-bis(diphenylphosphino)-1,1′-binaphthyl.

Further, the alkaline catalyst includes at least one of cesium carbonate, sodium tert-butoxide, potassium carbonate, sodium carbonate, sodium methoxide, sodium ethoxide and potassium tert-butoxide.

Further, the alkaline catalyst includes at least one of cesium carbonate, sodium tert-butoxide and potassium tert-butoxide.

Further, the first reaction solvent includes at least one of n-hexane, tetrahydrofuran, 1,4-dioxane, ethyl ether and toluene.

In order to achieve the above purpose, a method for preparing an aza[3.2.1]bridged ring substituted fluorescent dye is provided according to one aspect of the present disclosure. The preparation method includes: mixing a compound C, an aza[3.2.1]bridged ring compound and a second catalyst in a second reaction solvent to carry out a reaction so as to obtain the aza[3.2.1]bridged ring substituted fluorescent dye shown in Formula (II).

The compound C has a structure shown in Formula (VII) below:

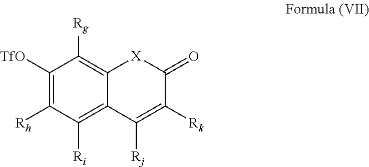

where R_{g}, Rₕ, Rᵢ, Rⱼ, Rₖ and X independently have the same meaning as defined in the first aspect.

Further, the second catalyst has the same meaning as the first catalyst.

Further, the second reaction solvent has the same meaning as the first reaction solvent.

In order to achieve the above purpose, a method for preparing an aza[3.2.1]bridged ring substituted fluorescent dye is provided according to one aspect of the present disclosure. The preparation method includes: step S31, mixing a compound D, an aza[3.2.1]bridged ring compound and a third catalyst in a third reaction solvent to carry out a reaction so as to obtain a compound E; step S32, mixing the compound E with an oxidizing agent in a fourth reaction solvent to carry out a reaction so as to obtain a compound F; and step S33, mixing the compound F with an HZ aqueous solution to carry out a reaction so as to obtain the aza[3.2.1]bridged ring substituted fluorescent dye shown in Formula (III).

The compound D has a structure shown in Formula (VIII) below:

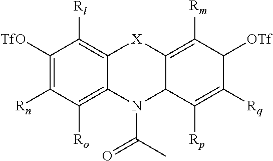

where Rₗ, Rₘ, Rₙ, Rₒ, Rₚ, R_{q}, X and Y independently have the same meaning as defined in the first aspect, and Z⁻ in the HZ aqueous solution has the same meaning as defined in the first aspect.

Further, the third catalyst has the same meaning as the first catalyst.

Further, the third reaction solvent has the same meaning as the first reaction solvent.

Further, in step S32, the fourth reaction solvent is a mixed solvent of dichloromethane and water, and the volume ratio of the dichloromethane to the water is (10-30):1.

Further, in step S32, the oxidizing agent includes at least one of 2,3-dichloro-5,6-dicyano-1,4-benzoquinone, potassium permanganate, hydrogen peroxide and m-chloroperoxybenzoic acid.

Further, the oxidizing agent is 2,3-dichloro-5,6-dicyano-1,4-benzoquinone.

Further, the molar ratio of the oxidizing agent to the compound E is (1.0-2.0):1.

In order to achieve the above purpose, use of the aza[3.2.1]bridged ring substituted fluorescent dye or the aza[3.2.1]bridged ring substituted fluorescent dye prepared by the method for preparing an aza[3.2.1]bridged ring substituted fluorescent dye in the fields of fluorescence analysis, fluorescence detection and fluorescence imaging as a fluorescent marker is provided according to one aspect of the present disclosure.

Further, use of the aza[3.2.1]bridged ring substituted fluorescent dye in fluorescence analysis, fluorescence detection, biomolecular labeling, immunofluorescence imaging, live-cell imaging, tissue imaging, in vivo imaging, single-molecule imaging and super-resolution fluorescence imaging as a fluorescent marker is provided.

According to use of the aza[3.2.1]bridged ring substituted fluorescent dye provided by the present disclosure in technical schemes of the present disclosure, by adopting an aza[3.2.1]bridged ring as a substituent of the fluorescent dye, not only are the brightness and photostability of the fluorescent dye obviously improved, but also the phenomenon of spectral blue shift caused by irradiation of the dye for a long time is completely avoided. Meanwhile, the water solubility of the fluorescent dye can be effectively improved, and good membrane permeability of the fluorescent dye is maintained.

### BRIEF DESCRIPTION OF THE DRAWINGS

Drawings attached to the specification that form a part of the present disclosure are used to provide further understanding of the present disclosure, and schematic embodiments of the present disclosure and description thereof are used to interpret the present disclosure and are not intended to constitute improper limitations to the present disclosure.

FIG. 1 shows absorption spectra of aza[3.2.1]bridged ring substituted fluorescent dyes according to Examples 1-4, 6, 7, 9, 18, 20 and 21 of the present disclosure;

FIG. 2 shows emission spectra of the aza[3.2.1]bridged ring substituted fluorescent dyes according to Examples 1-4, 6, 7, 9, 18, 20 and 21 of the present disclosure;

FIG. 3 shows comparison curves of differences of the photostability in orange channel according to Examples 3 and 10 as well as Comparative Examples 1~4 of the present disclosure;

FIG. 4 shows comparison curves of differences of the photostability in a far-red channel according to Example 7 and Comparative Example 5 of the present disclosure;

FIG. 5 shows a single molecule imaging photo of fixed U2OS cells according to Example 27 of the present disclosure;

FIG. 6 shows comparison of the single molecule brightness of fixed U2OS cells according to Example 27 and Comparative Examples 6 and 7 of the present disclosure;

FIG. 7 shows comparison of the photostability of fixed U2OS cells according to Example 27 and Comparative Examples 6 and 7 of the present disclosure;

FIG. 8 shows a confocal fluorescence imaging photo of labeled TMP-Halo-SNAP-GFP-H2B proteins in living Hela cells according to Example 27 of the present disclosure;

FIG. 9 shows a confocal fluorescence imaging photo of labeled TMP-Halo-SNAP-GFP-H2B proteins in living Hela cells according to Example 31 of the present disclosure;

FIG. 10 shows a confocal fluorescence imaging photo of labeled TMP-Halo-SNAP-GFP-H2B proteins in living Hela cells according to Example 34 of the present disclosure;

FIG. 11 shows comparison of the relative fluorescence intensity of labeled TMP-Halo-SNAP-GFP-H2B proteins in living Hela cells according to Example 27 and Comparative Examples 6 and 8 of the present disclosure;

FIG. 12 shows comparison of the relative fluorescence intensity of labeled TMP-Halo-SNAP-GFP-H2B proteins in living Hela cells according to Example 31 and Comparative Example 9 of the present disclosure;

FIG. 13 shows a confocal fluorescence imaging photo of labeled TMP-Halo-SNAP-GFP-H2B proteins in living Hela cells according to Example 28 of the present disclosure;

FIG. 14 shows comparison of the relative fluorescence intensity of labeled TMP-Halo-SNAP-GFP-H2B proteins in living Hela cells according to Example 28 and Comparative Example 10 of the present disclosure;

FIG. 15 shows a wash-free super-resolution fluorescence imaging photo of labeled Lifeact-Halo proteins in living Hela cells under an STED microscope according to Example 34 of the present disclosure;

FIG. 16 shows comparison of the photostability of labeled Lifeact-Halo proteins in living Hela cells under super-resolution fluorescence imaging conditions of an STED microscope according to Example 34 and Comparative Example 11 of the present disclosure.

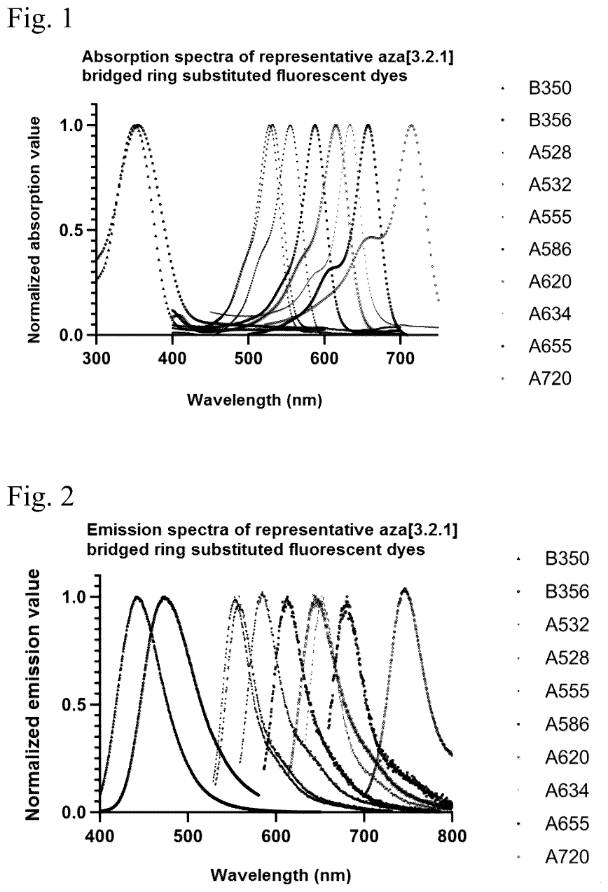

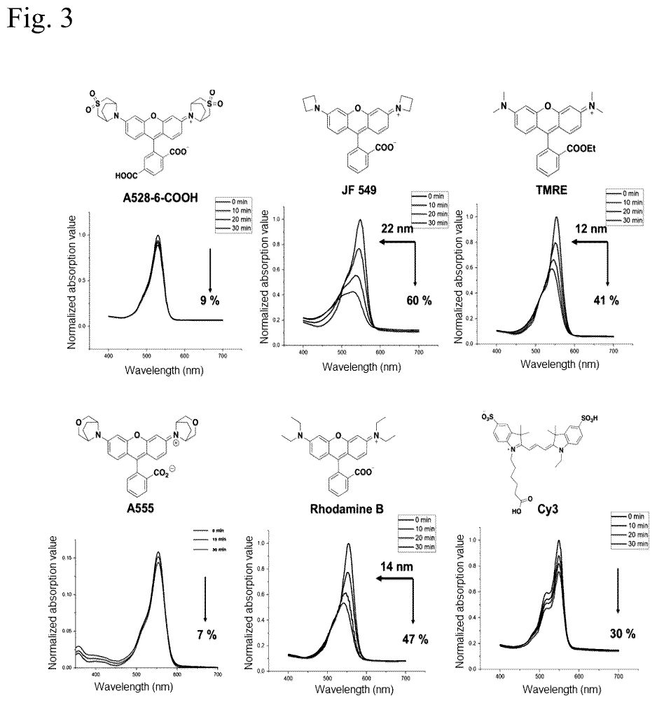

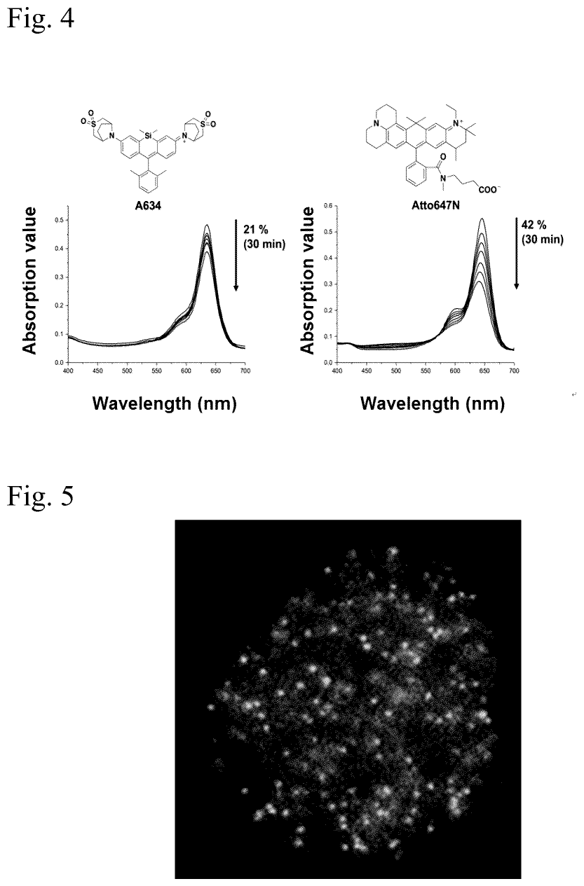

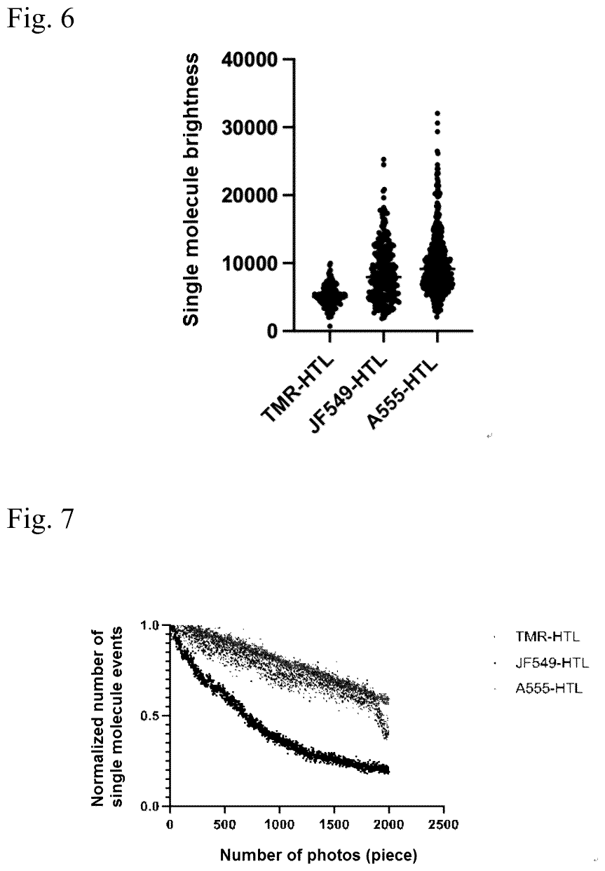

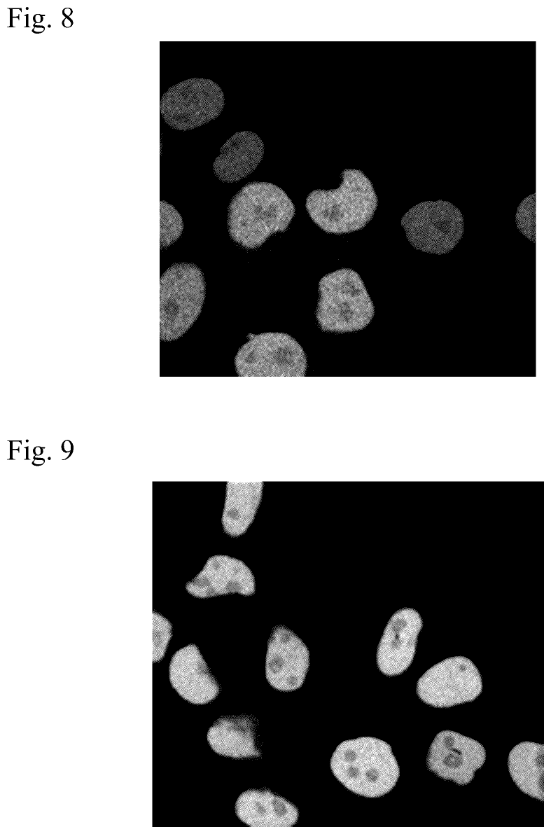

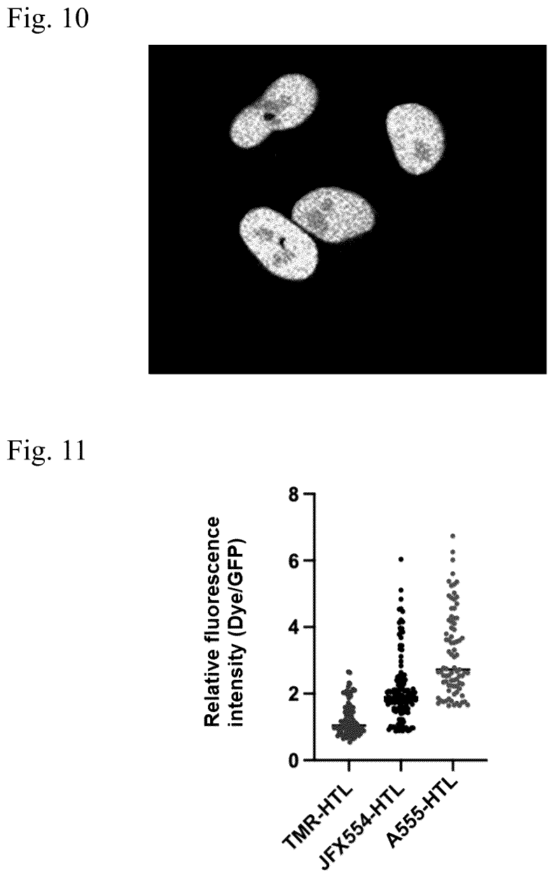

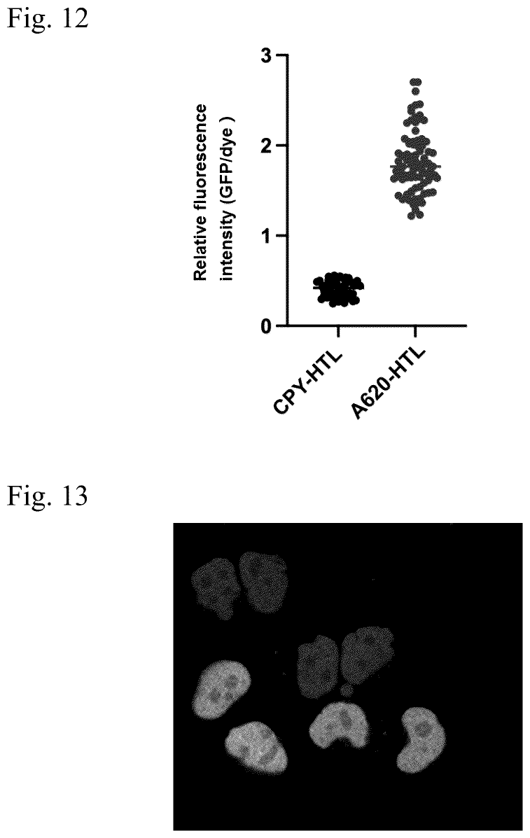

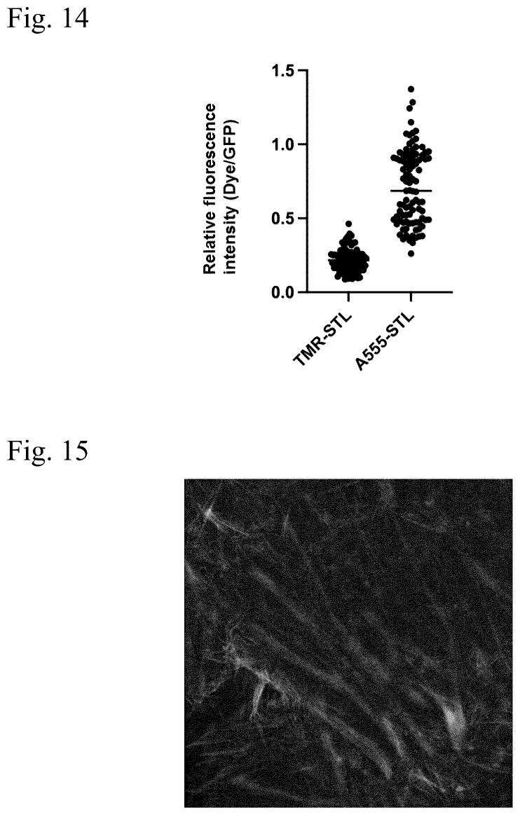

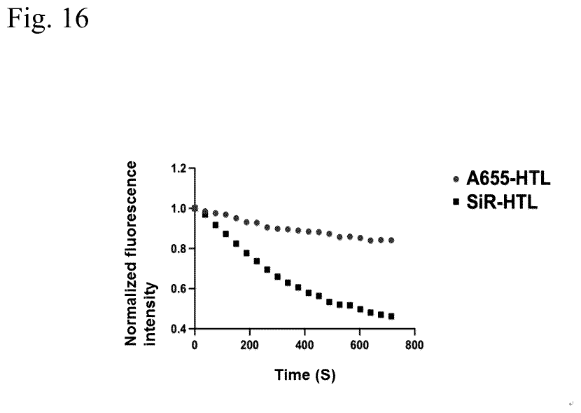

### DETAILED DESCRIPTION OF THE EMBODIMENTS

Notably, embodiments in the present disclosure and features in the embodiments may be combined with each other without conflict. The present disclosure is described in detail below with reference to the attached drawings and in conjunction with the embodiments.

As described in the background of the present disclosure, by introducing an azacyclobutane group into a matrix of a fluorescent dye in the prior art, the brightness of the fluorescent dye can be improved, but the fluorescent dye has poor photostability and no resistance to photooxidation dealkylation (spectral blue shift). In addition, by adopting a method of introducing azabicyclo[2.2.1]heptane into a matrix of a fluorescent dye to substitute rhodamine, the fluorescence quantum yield, photostability and resistance to photooxidation dealkylation (spectral blue shift) of the dye are improved, but the dye has poor water solubility and membrane permeability, so that use of the dye in living cell imaging is hindered. By introducing a sulfonic acid group into a matrix of a fluorescent dye, the water solubility of the dye can be improved, the antibody labeling efficiency is improved, and non-specific dyeing is reduced, but the dye is also prevented from permeating a cell membrane, so that the sulfonated dye cannot be used in living cells. In order to solve the problems, the present disclosure provides an aza[3.2.1]bridged ring substituted fluorescent dye and a preparation method and use thereof.

In a first typical embodiment of the present disclosure, an aza[3.2.1]bridged ring substituted fluorescent dye is provided. The aza[3.2.1]bridged ring substituted fluorescent dye has a structure shown in Formula (I), Formula (II) or Formula (III) below:

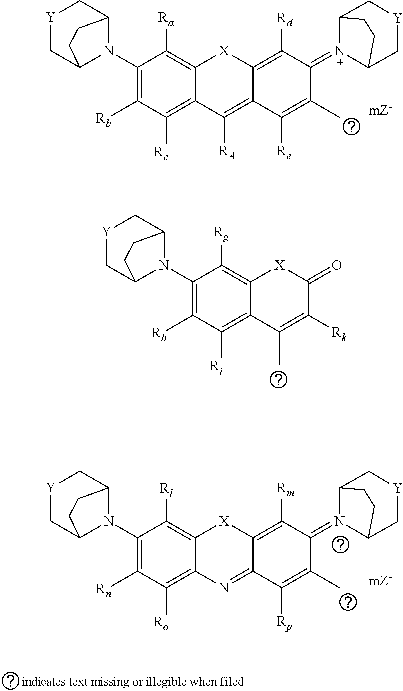

Rₐ, R_{b}, R_{c}, R_{d}, Rₑ, R_{f}, R_{g}, Rₕ, Rᵢ, Rⱼ, Rₖ, Rₗ, Rₘ, Rₙ, Rₒ, Rₚ and R_{q} are independently selected from hydrogen, halogen, carboxyl, substituted or unsubstituted C₁-C₂₀ alkyl, substituted or unsubstituted C₃-C₂₀ cycloalkyl, substituted or unsubstituted C₄-C₂₀ cycloalkylalkyl, C₄-C₂₀ alkylcycloalkyl, substituted or unsubstituted C₁-C₂₀ alkoxyl, substituted or unsubstituted C₁-C₂₀ ester group, and substituted or unsubstituted C₁-C₂₀ acylamino;
R_{A} is selected from substituted or unsubstituted C₆-C₄₀ aryl, substituted or unsubstituted C₆-C₄₀ aralkyl, and substituted or unsubstituted C₅-C₄₀ heteroaryl;
X is selected from oxygen, sulfur, substituted or unsubstituted C₁-C₂₀ alkylene,

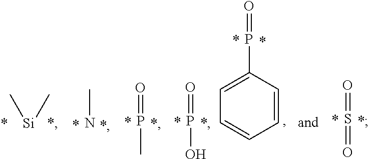

Y is selected from oxygen, sulfur, nitrogen, substituted or unsubstituted C₁-C₂₀ alkylene, substituted or unsubstituted C₁-C₂₀ alkylenoxyl, substituted or unsubstituted C₁-C₂₀ acyl, substituted or unsubstituted C₁-C₂₀ ester group,

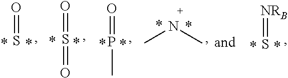

where R_{B} is selected from substituted or unsubstituted C₁-C₂₀ alkyl, substituted or unsubstituted C₁-C₂₀ alkoxyl, substituted or unsubstituted C₁-C₂₀ acyl, and substituted or unsubstituted C₁-C₂₀ ester group;
Z⁻ is selected from F⁻, Cl⁻, Br⁻, I⁻, OAc⁻, HSO₄⁻, H₂PO₄⁻, ClO₄⁻, CF₃COO⁻, CF₃SO₃⁻, CH₃SO₃⁻, NO₃⁻, C₅H₇O₅COO⁻, and PF₆⁻;
and in the Formula (I), the Z⁻ may exist in an ionic form and may also be connected with the R_{A} through a chemical bond, the Z⁻ may exist in the two forms simultaneously when m is equal to or greater than 2, and the m is an integer ranged from 1 to 3.

In the present disclosure, the “aryl” refers to a monocyclic ring or fused polycyclic ring derived from an aromatic hydrocarbon, and includes phenyl, biphenyl, bitriphenyl, naphthyl, binaphthyl, phenylnaphthyl, naphthylphenyl, fluorenyl, phenylfluorenyl, benzofluorenyl, dibenzofluorenyl, phenanthryl, phenylphenanthryl, anthryl, indenyl, bitriphenylenyl, pyrenyl, fused tetraphenyl, perylo, boryl, naphthonaphthyl, allenylfluorenyl and the like.

The “heteroaryl” refers to heteroaryl, and the heteroaryl refers to aryl containing at least one heteroatom selected from the group consisting of N, O and S as a main chain atom of a ring, which may be a monocyclic ring, such as furyl, thienyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazyl, pyridyl, pyrazinyl, pyrimidyl or pyridazinyl, or may be a fused ring condensed with at least one benzene ring, such as benzofuryl, benzothienyl, isobenzofuryl, dibenzofuryl, dibenzothienyl, benzoimidazolyl, benzothiazolyl, benzoisothiazolyl, benzoisoxazolyl, quinolyl, isoquinolyl, cinolinyl, quinazolinyl, quinoxalinyl, carbazolyl, phenoxazolyl, phenanthridinyl, benzodiacenaphthenyl or dihydroacridinyl.

The “substituted” in the “substituted or unsubstituted” indicates that a hydrogen atom in a functional group is substituted with another atom or group (namely, substituent). The substituent is independently select from at least one of the following groups: deuterium, halogen, C₁-C₁₀ alkyl, C₁-C₁₀ alkoxyl, C₆-C₁₀ aryl, C₅-C₁₀ heteroaryl, C₅-C₁₀ heteroaryl substituted with C₆-C₁₀ aryl, imidazolyl, benzoimidazolyl, C₃-C₁₀ cycloalkyl, C₅-C₇ heterocycloalkyl, tri-(C₁-C₁₀) alkylsilyl, tri-(C₁-C₁₀) arylsilyl, di-(C₁-C₁₀) alkyl-(C₆-C₁₀) arylsilyl, C₁-C₁₀ alkyl-di-(C₆-C₁₀) arylsilyl, C₂-C₁₀ alkenyl, C₂-C₃₀ alkynyl, cyano, di-(C₁-C₃₀) alkylamino, di-(C₆-C₃₀) arylboryl, di-(C₁-C₃₀) alkylboryl, C₁-C₃₀ alkyl, C₆-C₁₀ arylC₁-C₁₀ alkyl, C₁-C₁₀ alkyl C₆-C₁₀ aryl, carboxyl, nitro, hydroxyl,

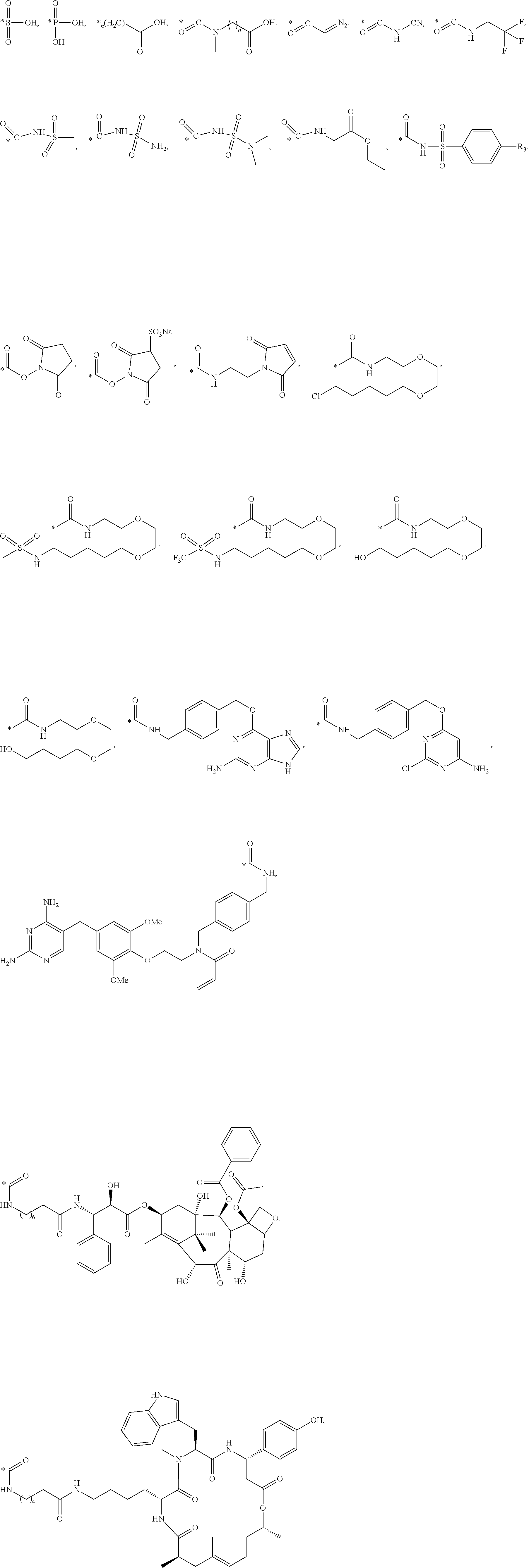

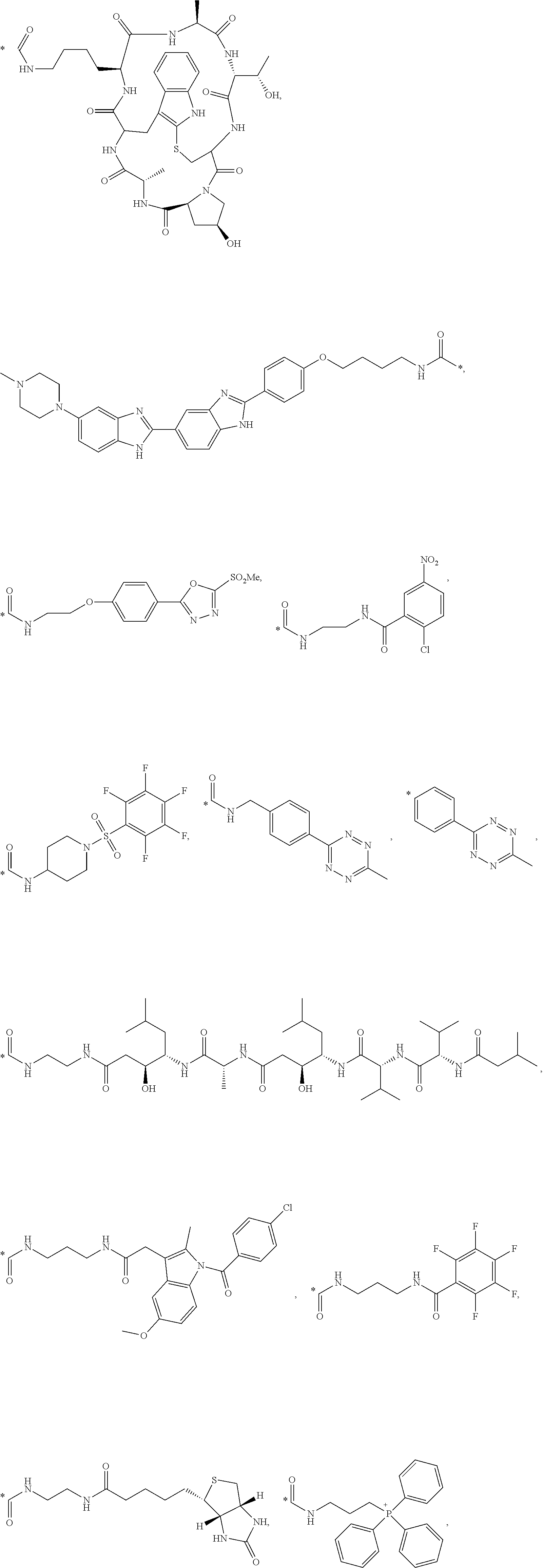

or any other functional groups used for biological coupling or subcellular localization, where R_{D} is selected from hydrogen, halogen, cyano, substituted or unsubstituted C₁-C₄ alkyl, and substituted or unsubstituted C₁-C₆ alkoxyl, and n is a positive integer ranged from 1 to 6. In addition, the number of carbon atoms described herein may be ranged from a lower limit to an upper limit. For example, C₆-C₁₀ indicates that the number of carbon atoms may be 6, 7, 8, 9 or 10.

In the present disclosure, “*” represents a connecting bond.

According to use of the aza[3.2.1]bridged ring substituted fluorescent dye provided by the present disclosure in technical schemes of the present disclosure, by adopting an aza[3.2.1]bridged ring as a substituent of the fluorescent dye, not only are the brightness and photostability of the fluorescent dye obviously improved, but also the phenomenon of spectral blue shift caused by irradiation of the dye for a long time is completely avoided. Meanwhile, the water solubility of the fluorescent dye can be effectively improved, and good membrane permeability of the fluorescent dye is maintained.

In order to further improve properties of the aza[3.2.1]bridged ring substituted fluorescent dye, preferably, the Rₐ, the R_{b}, the R_{c}, the Rₐ, the Rₑ, the R_{f}, the R_{g}, the Rₕ, the Rᵢ, the Rⱼ, the Rₖ, the Rₗ, the Rₘ, the Rₙ, the Rₒ, the Rₚ and the R_{q} are independently selected from hydrogen, halogen, carboxyl, substituted or unsubstituted C₁-C₁₀ alkyl, substituted or unsubstituted C₃-C₁₀ cycloalkyl, substituted or unsubstituted C₄-C₁₀ cycloalkylalkyl, C₄-C₁₀ alkylcycloalkyl, substituted or unsubstituted C₁-C₁₀ alkoxyl, substituted or unsubstituted C₁-C₁₀ ester group, and substituted or unsubstituted C₁-C₁₀ acylamino.

In some embodiments, preferably, the R_{A} is selected from substituted or unsubstituted C₆-C₃₀ aryl, substituted or unsubstituted C₆-C₃₀ aralkyl, and substituted or unsubstituted C₅-C₃₀ heteroaryl.

In other embodiments, preferably, the X is selected from oxygen, sulfur, substituted or unsubstituted C₁-C₁₀ alkyl,

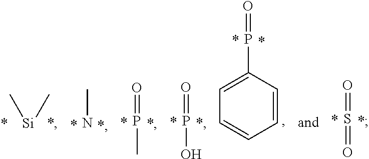

preferably, the Y is selected from oxygen, sulfur, nitrogen, substituted or unsubstituted C₁-C₁₀ alkyl, substituted or unsubstituted C₁-C₁₀ alkoxyl, substituted or unsubstituted C₁-C₁₀ acyl, substituted or unsubstituted C₁-C₁₀ ester group,

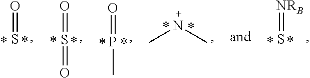

where the R_{B} is selected from substituted or unsubstituted C₁-C₂₀ alkyl, substituted or unsubstituted C₁-C₂₀ alkoxyl, substituted or unsubstituted C₁-C₂₀ acyl, and substituted or unsubstituted C₁-C₂₀ ester group.

In some other embodiments, preferably, the Z⁻ is selected from F⁻, Cl⁻, Br⁻, I⁻, OAc⁻, HSO₄⁻, H₂PO₄⁻, ClO₄⁻, CF₃COO⁻, CF₃SO₃⁻, CH₃SO₃⁻, NO₃⁻, C₅H₇O₅COO⁻, and PF₆⁻; m is a positive integer ranged from 1 to 3; and OAc⁻ represents an acetate ion.

In order to further improve properties of the aza[3.2.1]bridged ring substituted fluorescent dye, preferably, the aza[3.2.1]bridged ring substituted fluorescent dye has a structure shown in Formula (IV) below:

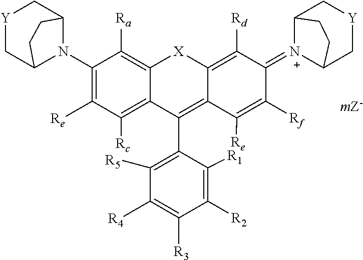

R₁ and R₅ are independently selected from hydrogen, carboxyl, halogen, substituted or unsubstituted C₁-C₆ alkyl, substituted or unsubstituted C₁-C₆ alkoxyl, nitro, amino, hydroxyl,

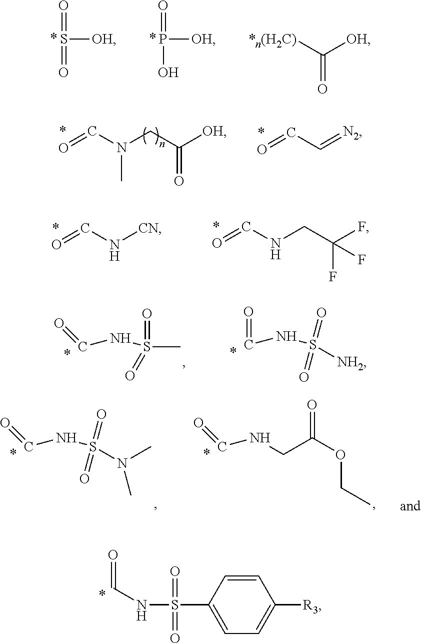

where R_{D} is selected from hydrogen, halogen, cyano, substituted or unsubstituted C₁-C₄ alkyl, and substituted or unsubstituted C₁-C₆ alkoxyl, and n is an integer ranged from 1 to 6.

R₂, R₃ and R₄ are independently selected from hydrogen, halogen, carboxyl, substituted or unsubstituted C₁-C₁₀ alkyl, substituted or unsubstituted C₃-C₁₀ cycloalkyl, substituted or unsubstituted C₄-C₁₀ cycloalkylalkyl, C₄-C₁₀ alkylcycloalkyl, substituted or unsubstituted C₁-C₁₀ alkoxyl, substituted or unsubstituted C₁-C₁₀ ester group,

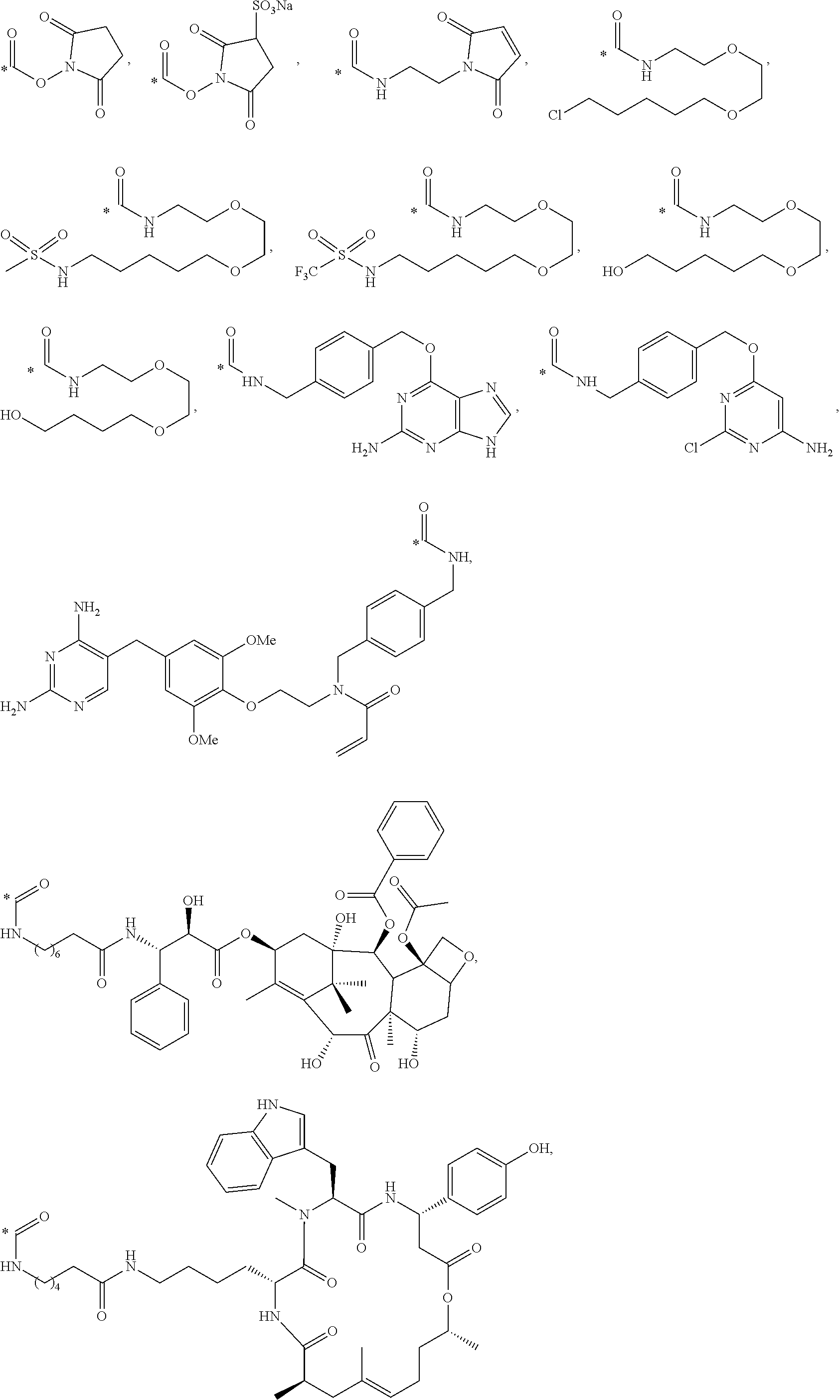

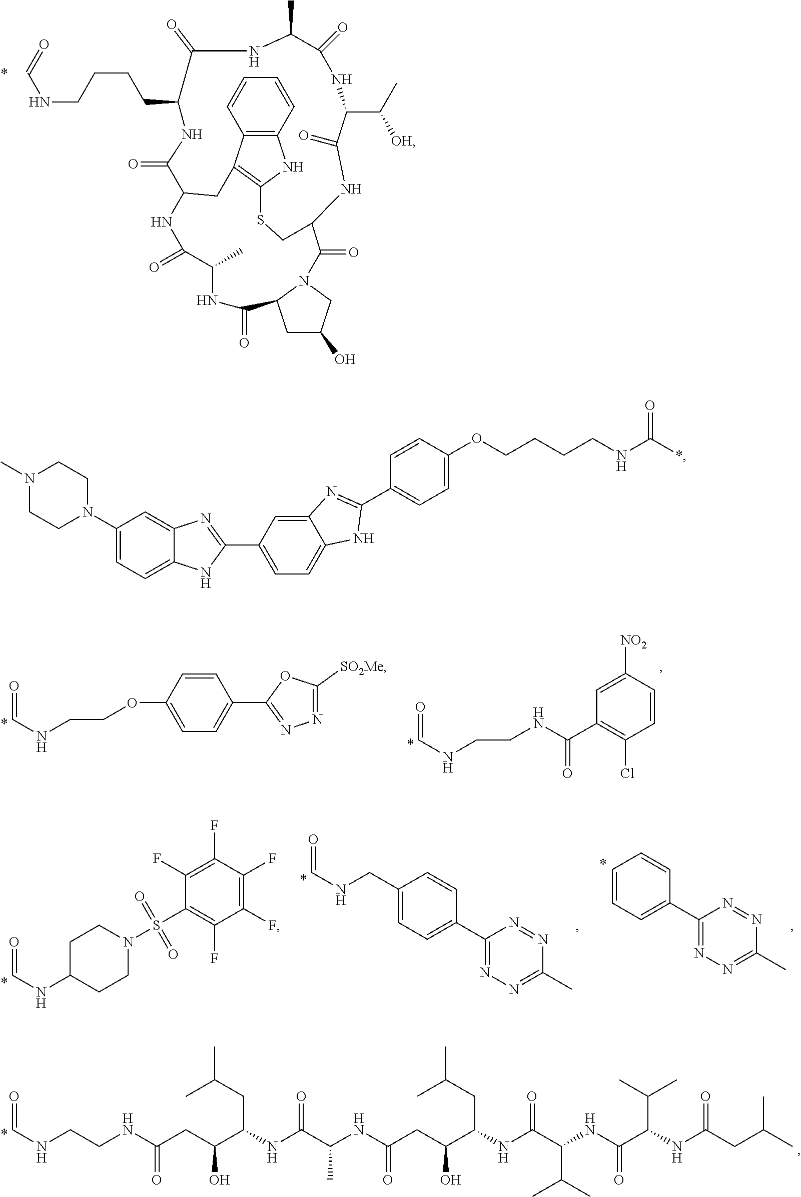

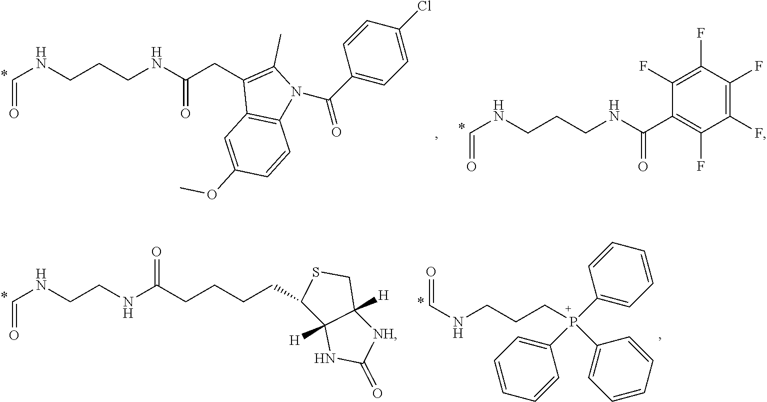

or any other functional groups used for biological coupling or subcellular localization.

In some specific embodiments of the present disclosure, the aza[3.2.1]bridged ring substituted fluorescent dye is selected from at least one of the following formulas IA1-IA3, IIA1-IIA3, IIIA1-IIIA3, IB1-IB3, IC1-IC3, ID1-ID3, IE1-IE3, IF1-IF3, and IG1-IG3, where Ph is phenyl:

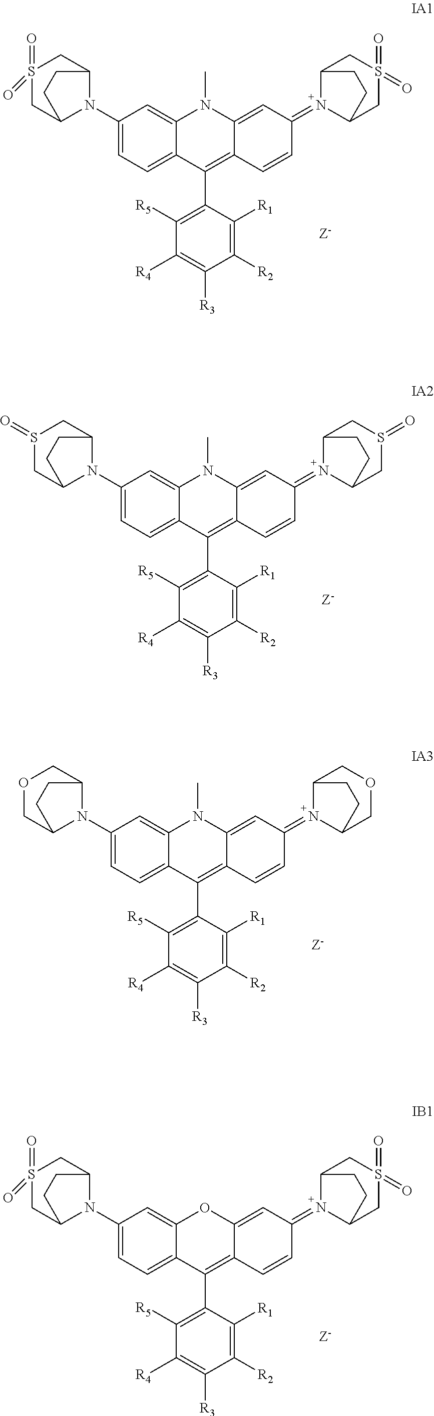

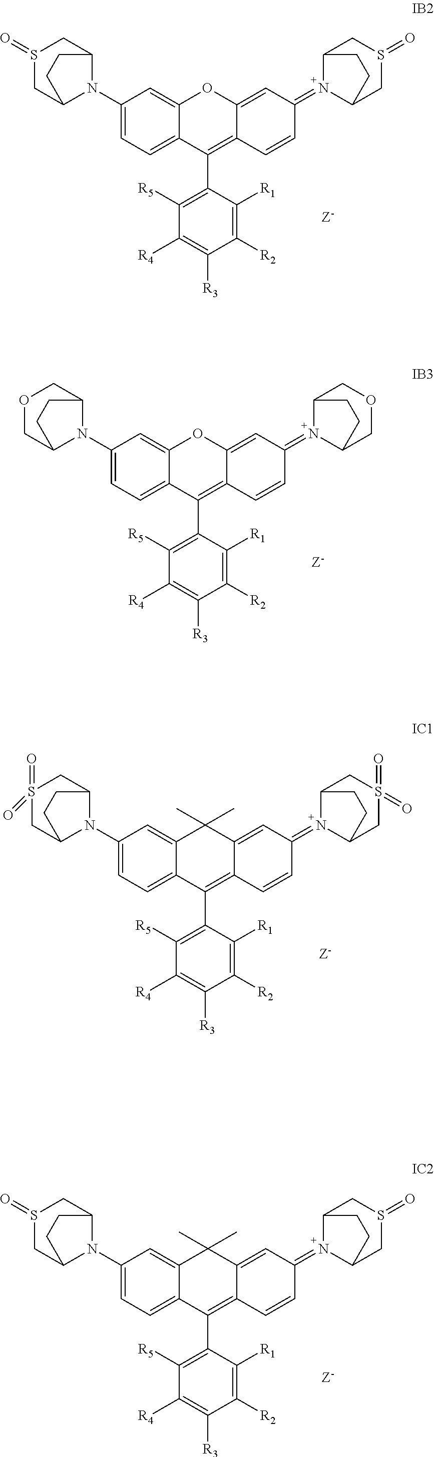

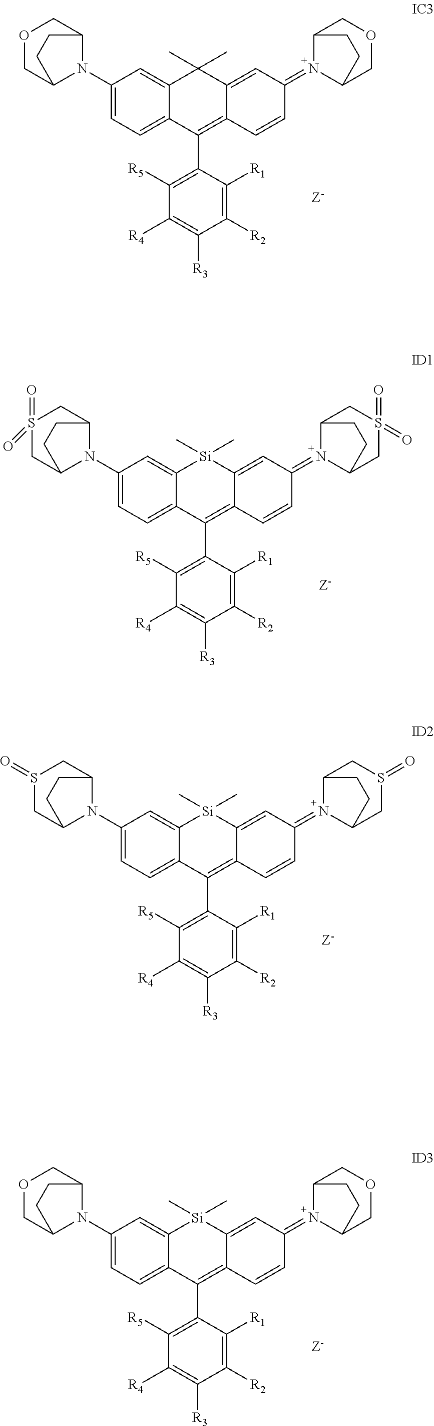

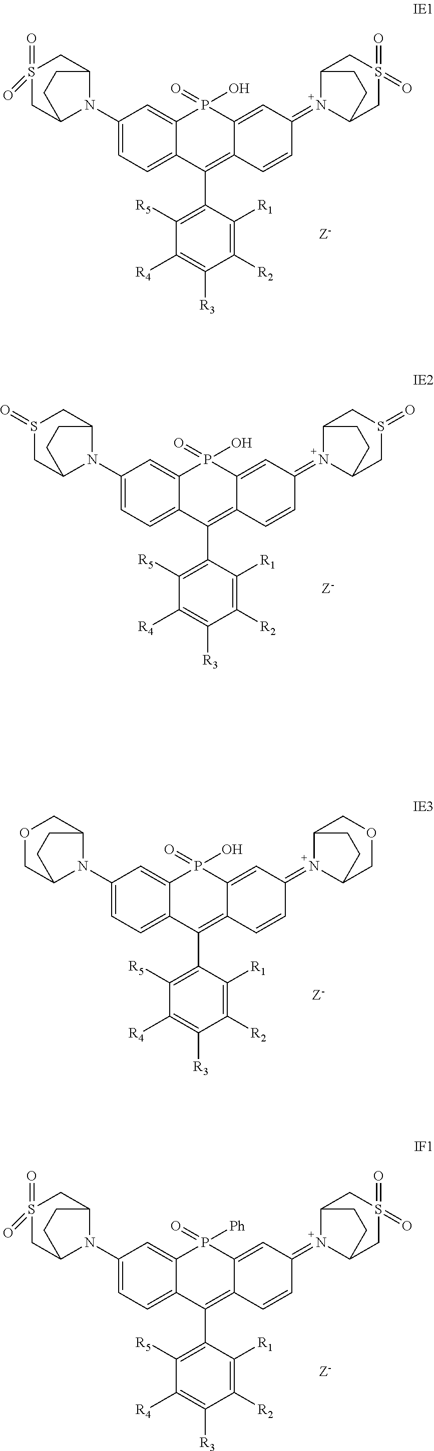

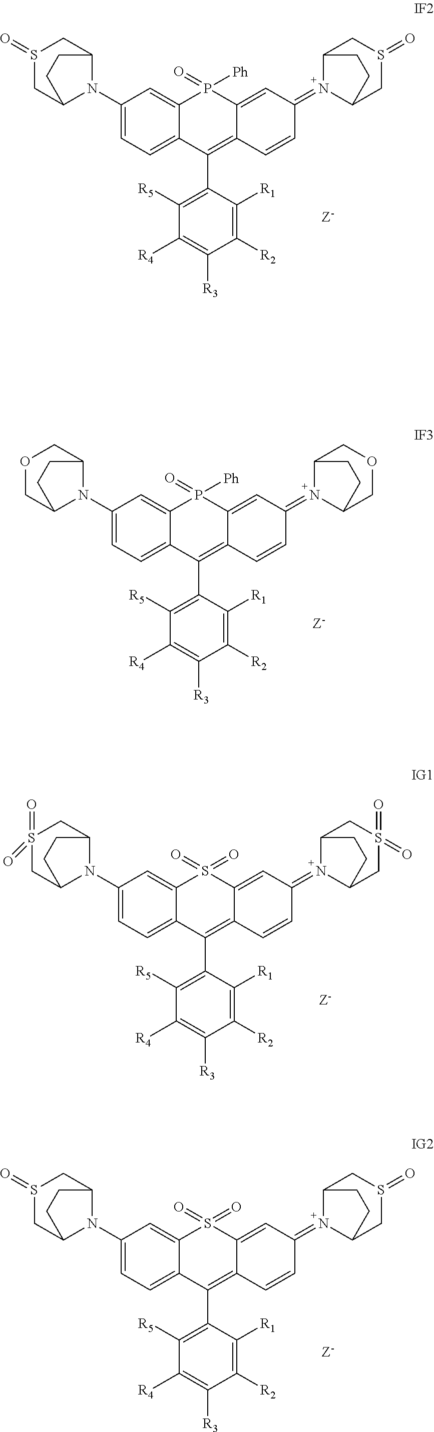

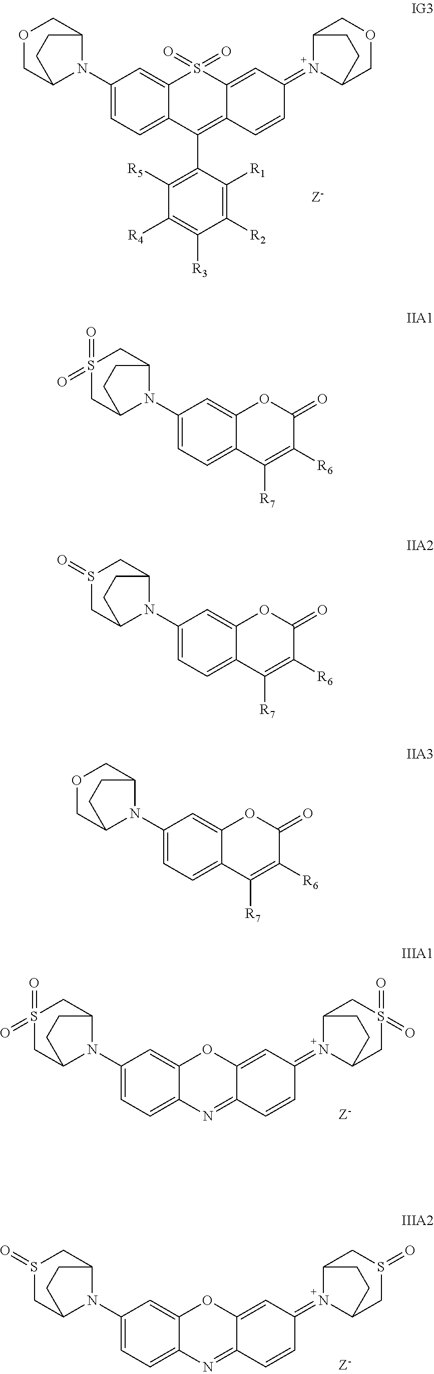

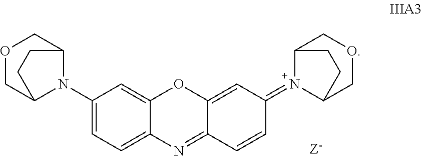

In other specific embodiments of the present disclosure, the aza[3.2.1]bridged ring substituted fluorescent dye is selected from at least one of the following formulas A528, A532, A555, A586, A591, A620, A634, A640, A655, A720, A586-2Me, A586-2Me-5-COOH), A528-6-COOH, A532-6-COOH), A555-6-COOH), A586-6-COOH), A591-6-COOH), A620-6-COOH), A634-5-COOH), A640-6-COOH), A655-6-COOH), A528-HTL, A532-HTL, A555-HTL, A586-HTL, A620-HTL, A655-HTL, A555-STL, A528-NHS, A620-STL, A655-STL, A634-NHS, A586-NHS, A591-HTL, A640-HTL, A555-TMP3, A620-TMP3, A655-TMP3, A655-4-Hoechst, A591-TMP3, A640-TMP3, A591-STL, A640-STL, A528-NCN-HTL, A555-NCN-HTL, A620-NCN-HTL, B350, B356, C622, and C654:

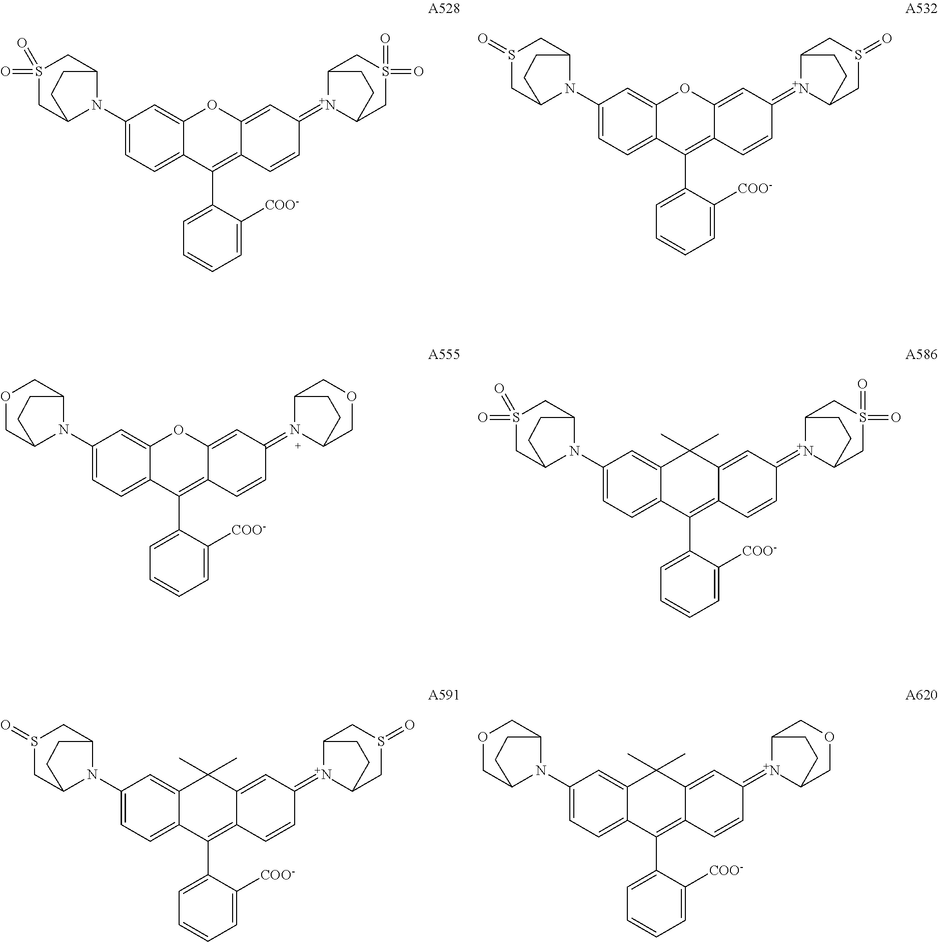

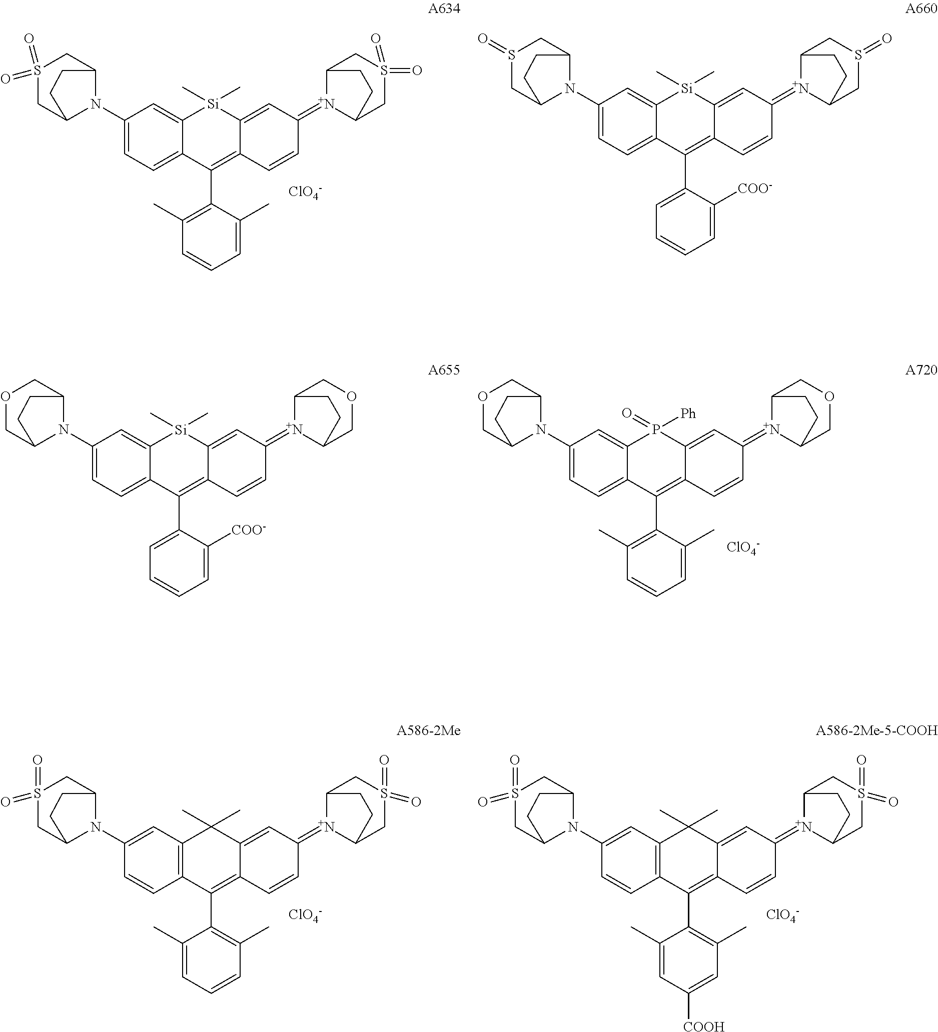

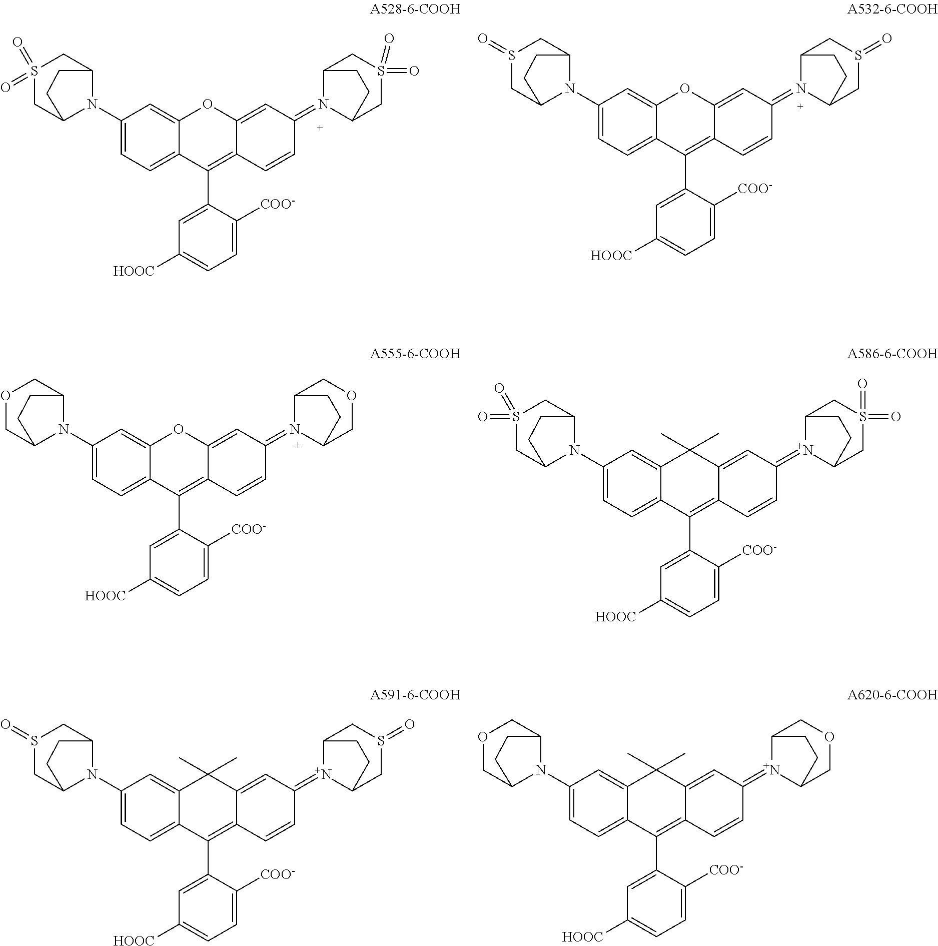

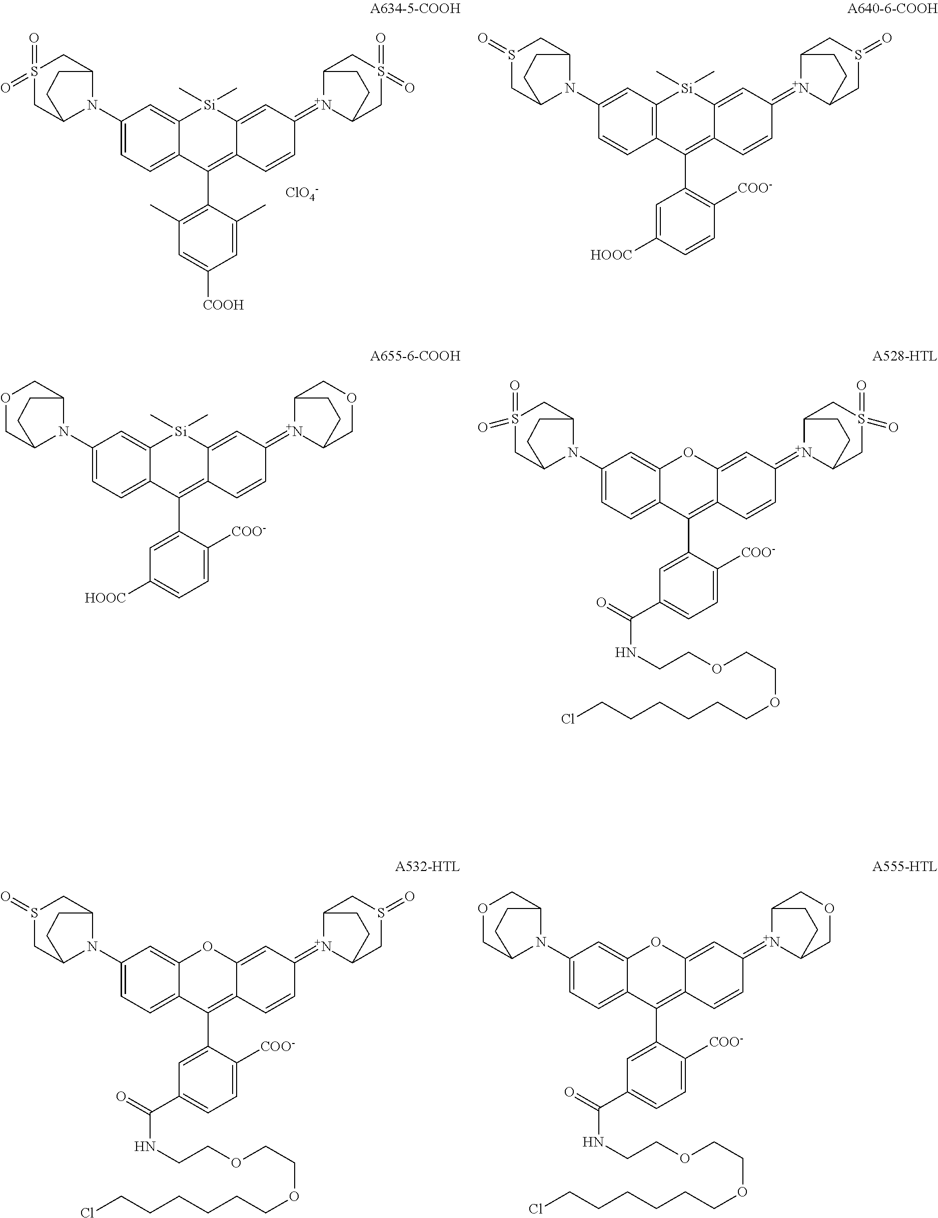

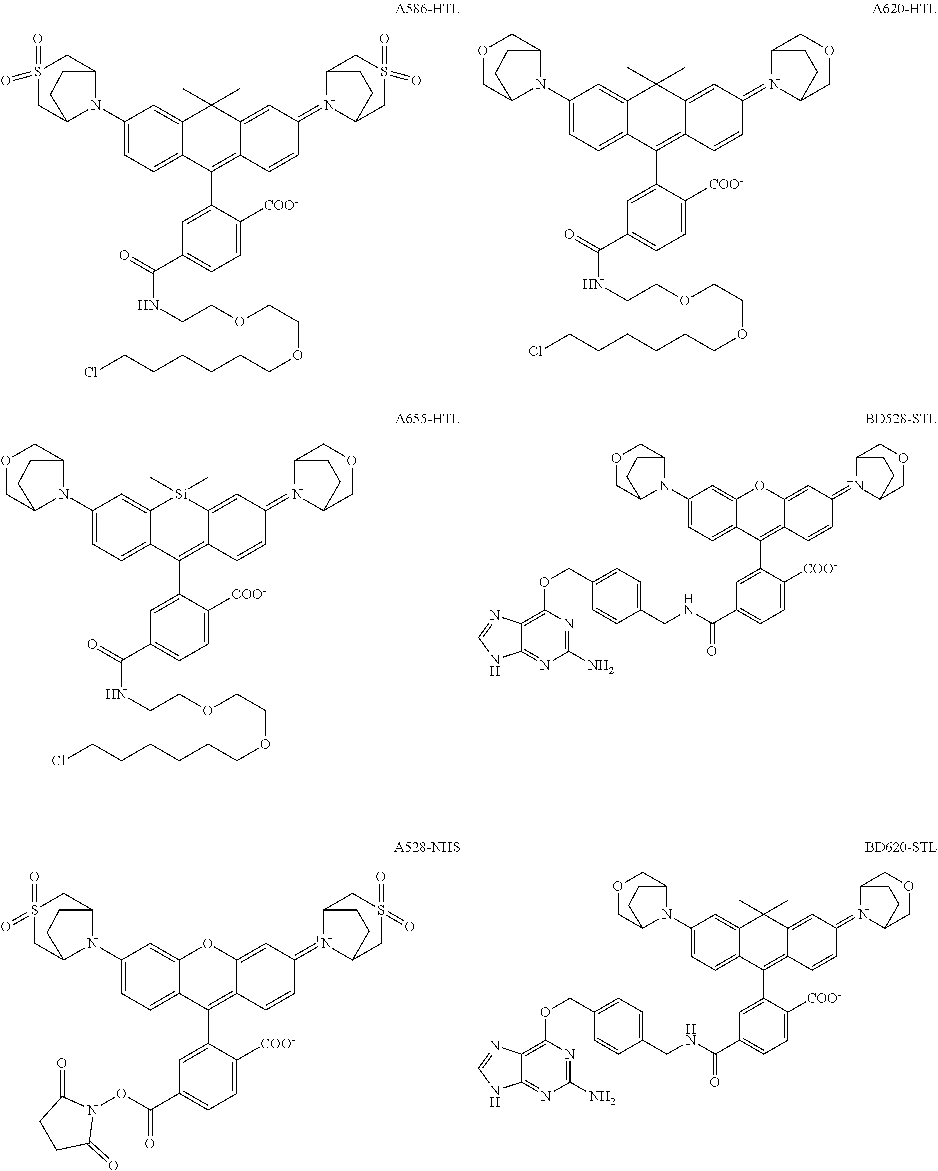

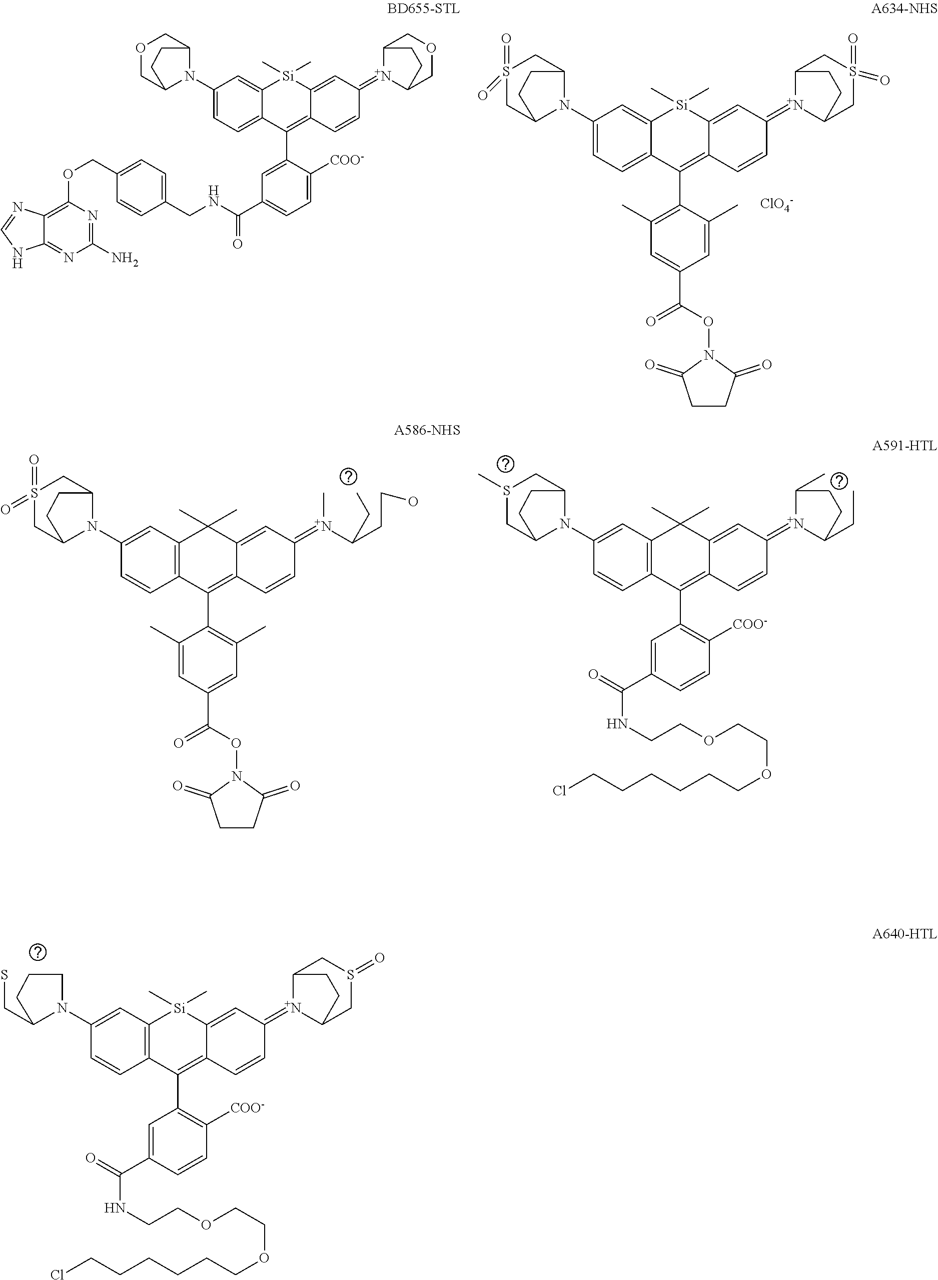

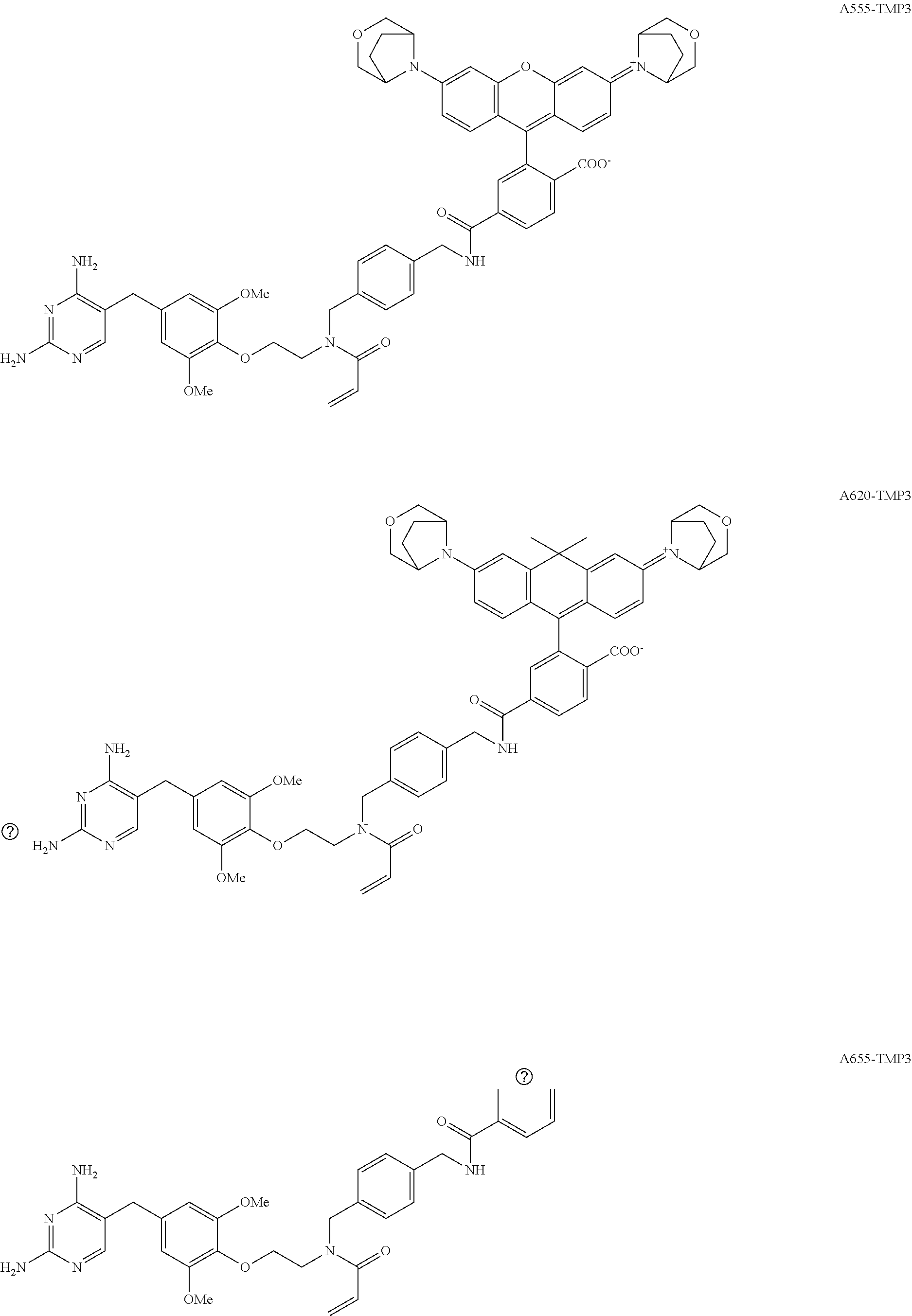

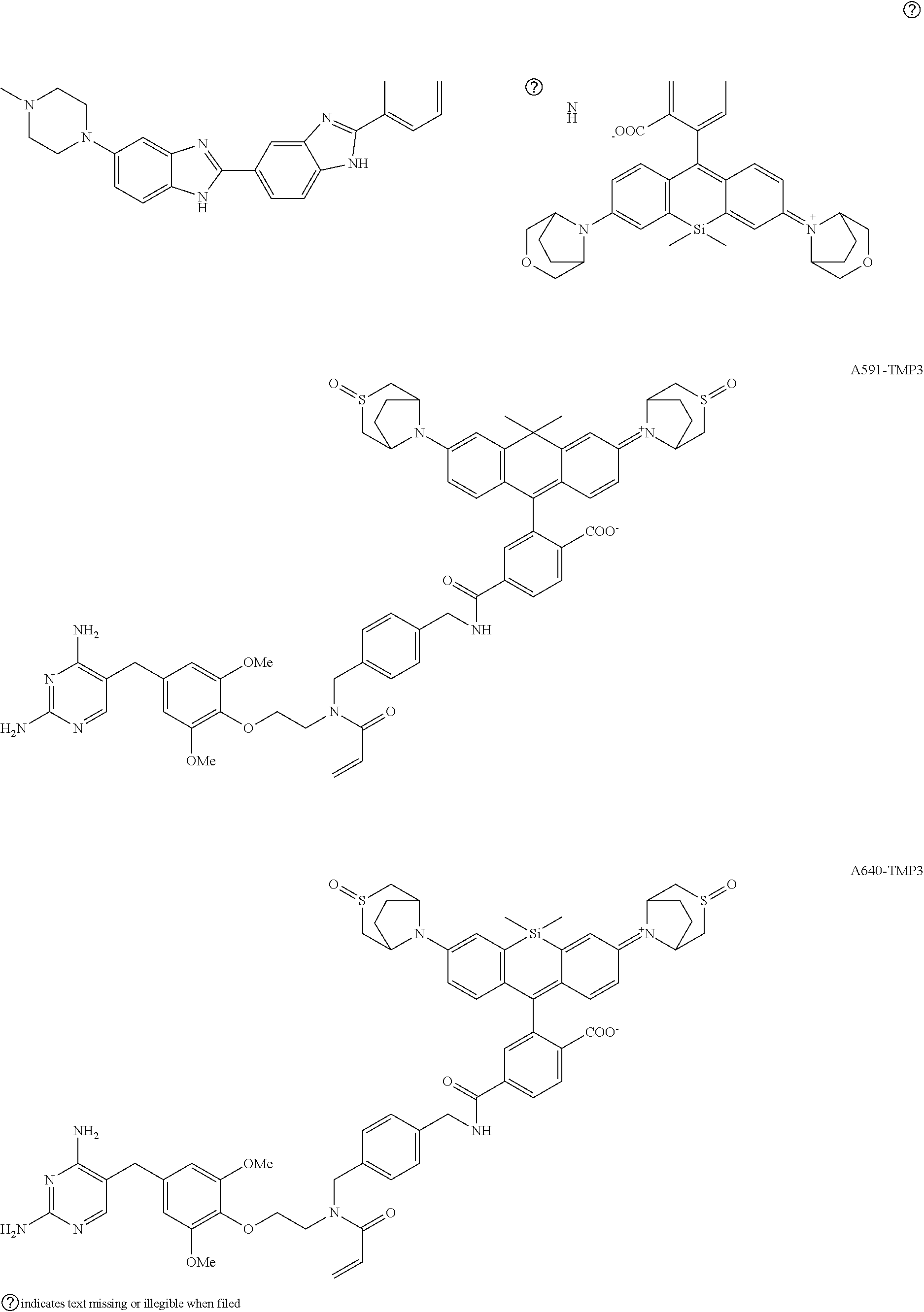

1-10. (canceled)

11. An aza[3.2.1]bridged ring substituted fluorescent dye, wherein the aza[3.2.1]bridged ring substituted fluorescent dye has a structure shown in Formula (I), Formula (II) or Formula (III) below: 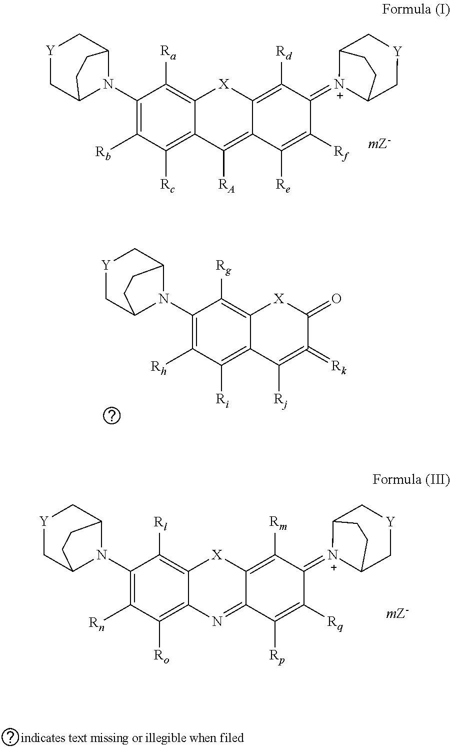
Rₐ, R_{b}, R_{c}, Rₐ, Rₑ, R_{f}, R_{g}, Rₕ, Rᵢ, Rⱼ, Rₖ, Rₗ, Rₘ, Rₙ, Rₒ, Rₚ and R_{q} are independently selected from hydrogen, halogen, carboxyl, substituted or unsubstituted C₁-C₂₀ alkyl, substituted or unsubstituted C₃-C₂₀ cycloalkyl, substituted or unsubstituted C₄-C₂₀ cycloalkylalkyl, C₄-C₂₀ alkylcycloalkyl, substituted or unsubstituted C₁-C₂₀ alkoxyl, substituted or unsubstituted C₁-C₂₀ ester group, and substituted or unsubstituted C₁-C₂₀ acylamino;
R_{A} is selected from substituted or unsubstituted C₆-C₄₀ aryl, substituted or unsubstituted C₆-C₄₀ aralkyl, and substituted or unsubstituted C₅-C₄₀ heteroaryl;
X is selected from oxygen, sulfur, substituted or unsubstituted C₁-C₂₀ alkylene, 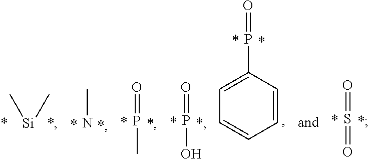
Y is selected from oxygen, sulfur, nitrogen, substituted or unsubstituted C₁-C₂₀ alkylene, substituted or unsubstituted C₁-C₂₀ alkylenoxyl, substituted or unsubstituted C₁-C₂₀ acyl, substituted or unsubstituted C₁-C₂₀ ester group, 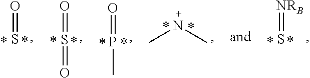
wherein R_{B} is selected from substituted or unsubstituted C₁-C₂₀ alkyl, substituted or unsubstituted C₁-C₂₀ alkoxyl, substituted or unsubstituted C₁-C₂₀ acyl, and substituted or unsubstituted C₁-C₂₀ ester group;
Z⁻ is selected from F⁻, Cl⁻, Br⁻, I⁻, OAc⁻, HSO₄⁻, H₂PO₄⁻, ClO₄⁻, CF₃COO⁻, CF₃SO₃⁻, CH₃ SO₃⁻, NO₃⁻, C₅H₇O₅COO⁻, and PF₆⁻; and m is an integer ranged from 1 to 3.

12. The aza[3.2.1]bridged ring substituted fluorescent dye according to claim 11, wherein the Rₐ, the R_{b}, the R_{c}, the Rₐ, the Rₑ, the R_{f}, the R_{g}, the Rₕ, the Rᵢ, the Rⱼ, the Rₖ, the Rᵢ, the Rₘ, the Rₙ, the Rₒ, the Rₚ and the R_{q} are independently selected from hydrogen, halogen, carboxyl, substituted or unsubstituted C₁-C₁₀ alkyl, substituted or unsubstituted C₃-C₁₀ cycloalkyl, substituted or unsubstituted C₄-C₁₀ cycloalkylalkyl, C₄-C₁₀ alkylcycloalkyl, substituted or unsubstituted C₁-C₁₀ alkoxyl, substituted or unsubstituted C₁-C₁₀ ester group, and substituted or unsubstituted C₁-C₁₀ acylamino;
and/or, the R_{A} is selected from substituted or unsubstituted C₆-C₃₀ aryl, substituted or unsubstituted C₆-C₃₀ aralkyl, and substituted or unsubstituted C₅-C₃₀ heteroaryl;
and/or, the X is selected from oxygen, sulfur, substituted or unsubstituted C₁-C₁₀ alkyl, 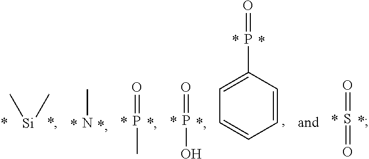
and/or, the Y is selected from oxygen, sulfur, nitrogen, substituted or unsubstituted C₁-C₁₀ alkyl, substituted or unsubstituted C₁-C₁₀ alkoxyl, substituted or unsubstituted C₁-C₁₀ acyl, substituted or unsubstituted C₁-C₁₀ ester group, 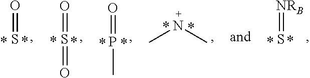
wherein the R_{B} is selected from substituted or unsubstituted C₁-C₂₀ alkyl, substituted or unsubstituted C₁-C₂₀ alkoxyl, substituted or unsubstituted C₁-C₂₀ acyl, and substituted or unsubstituted C₁-C₂₀ ester group;
and/or, the Z⁻ is selected from F⁻, Cl⁻, Br⁻, I⁻, OAc⁻, HSO₄⁻, H₂PO₄⁻, ClO₄⁻, CF₃COO⁻, CF₃SO₃⁻, CH₃SO₃⁻, NO₃⁻, C₅H₇O₅COO⁻, and PF₆⁻;
and/or, the m is an integer ranged from 1 to 3.

13. The aza[3.2.1]bridged ring substituted fluorescent dye according to claim 11, wherein the aza[3.2.1]bridged ring substituted fluorescent dye shown in Formula (I) has a structure shown in Formula (IV) below: 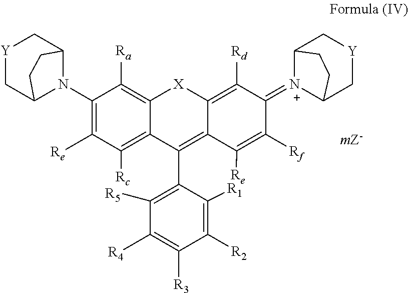
R₁ and R₅ are independently selected from hydrogen, carboxyl, halogen, substituted or unsubstituted C₁-C₆ alkyl, substituted or unsubstituted C₁-C₆ alkoxyl, nitro, amino, hydroxyl, 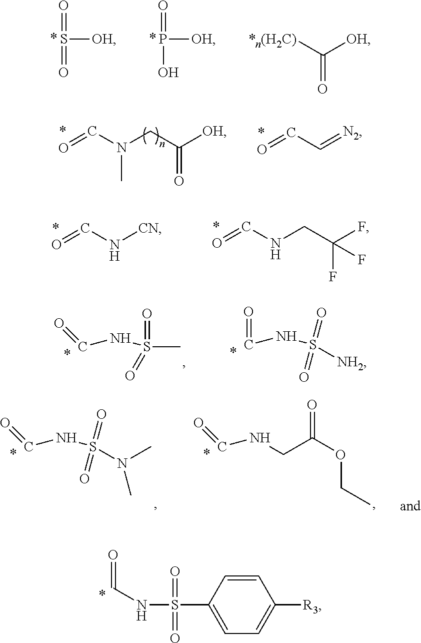
wherein R_{D} is selected from hydrogen, halogen, cyano, substituted or unsubstituted C₁-C₆ alkyl, and substituted or unsubstituted C₁-C₆ alkoxyl, and n is an integer ranged from 1 to 6; and R₂, R₃ and R₄ are independently selected from hydrogen, halogen, carboxyl, substituted or unsubstituted C₁-C₁₀ alkyl, substituted or unsubstituted C₃-C₁₀ cycloalkyl, substituted or unsubstituted C₄-C₁₀ cycloalkylalkyl, C₄-C₁₀ alkylcycloalkyl, substituted or unsubstituted C₁-C₁₀ alkoxyl, substituted or unsubstituted C₁-C₁₀ ester group,

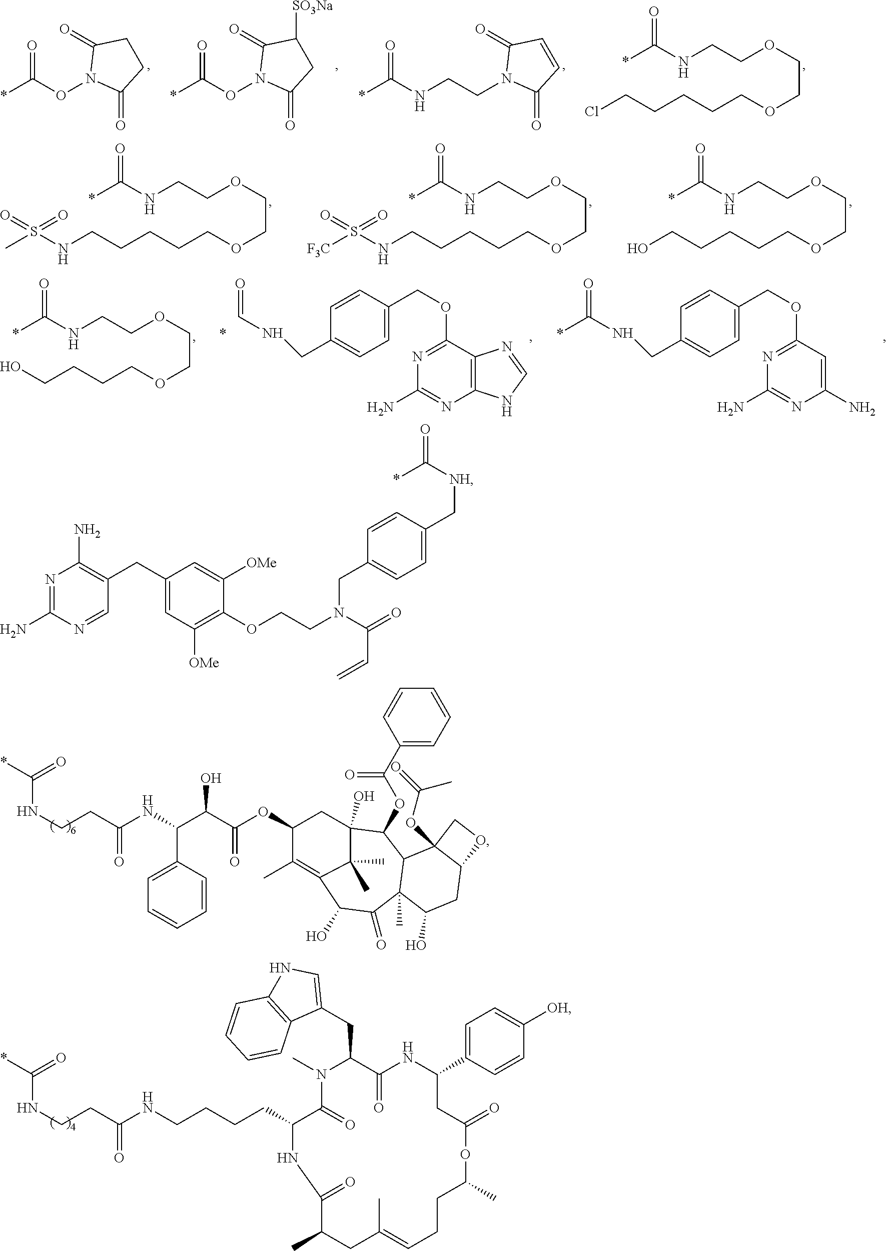

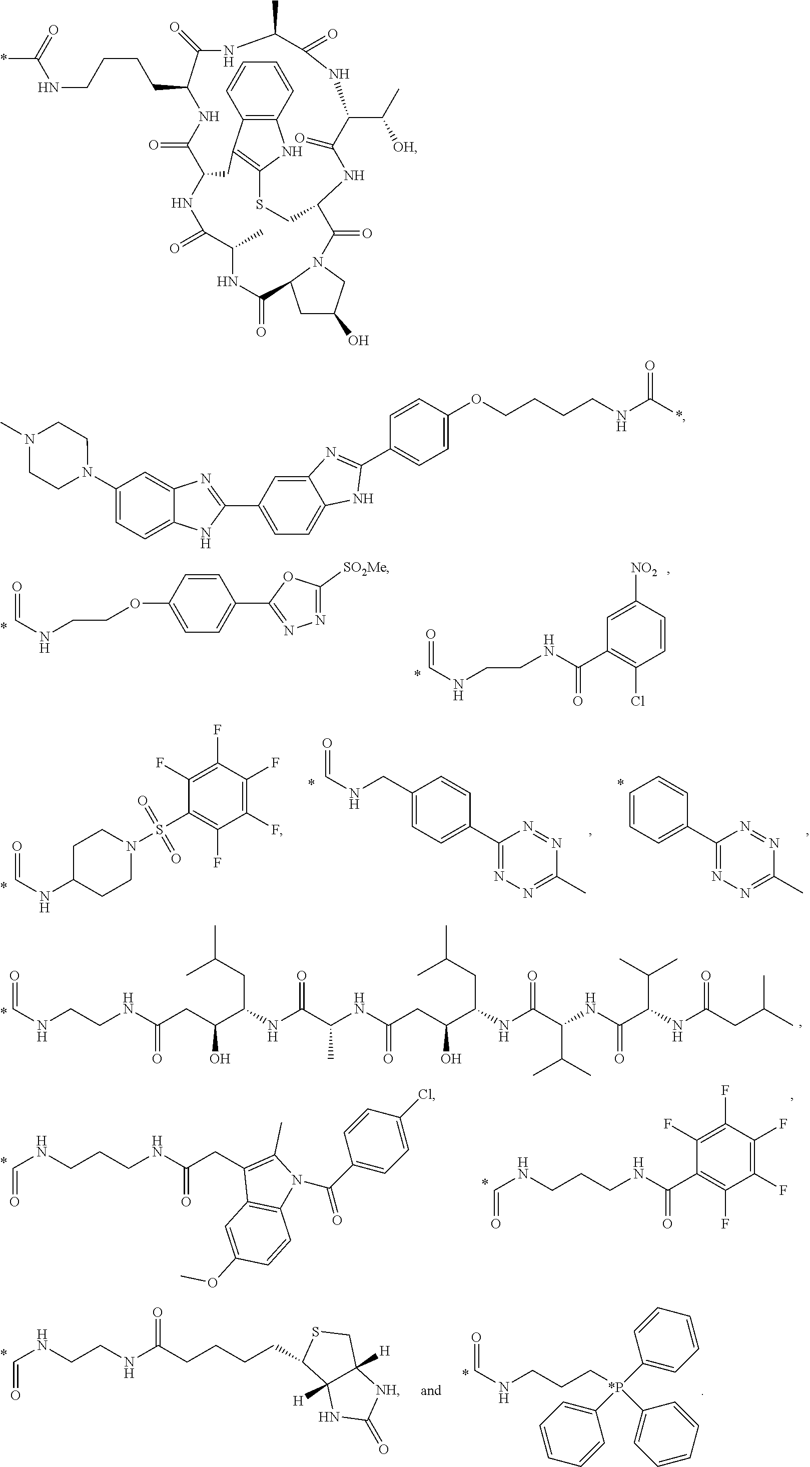

14. The aza[3.2.1]bridged ring substituted fluorescent dye according to claim 13, wherein the aza[3.2.1]bridged ring substituted fluorescent dye is selected from at least one of the following formulas IA1-IA3, IIA1-IIA3, IIIA1-IIIA3, IB1-IB3, IC1-IC3, ID1-ID3, IE1-IE3, IF1-IF3, and IG1-IG3:

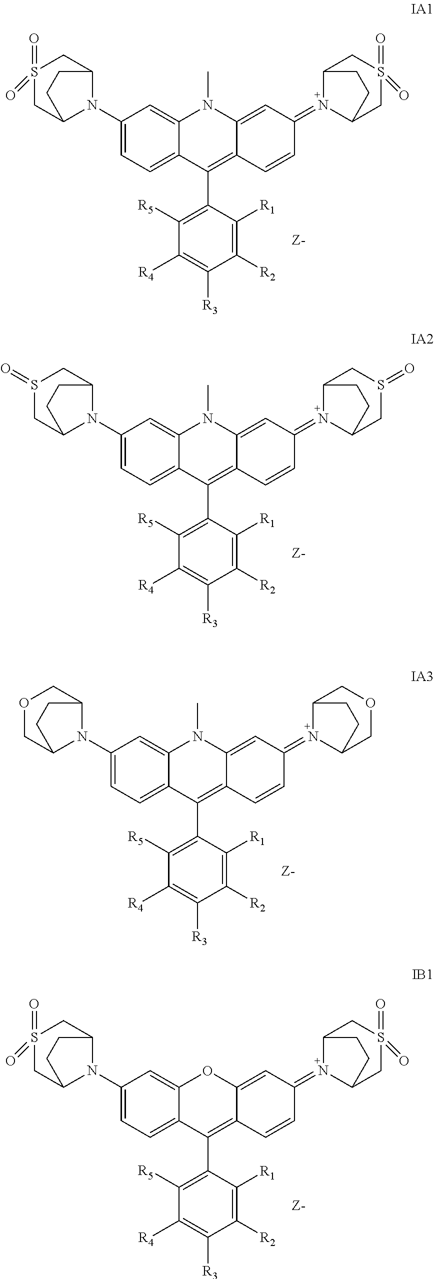

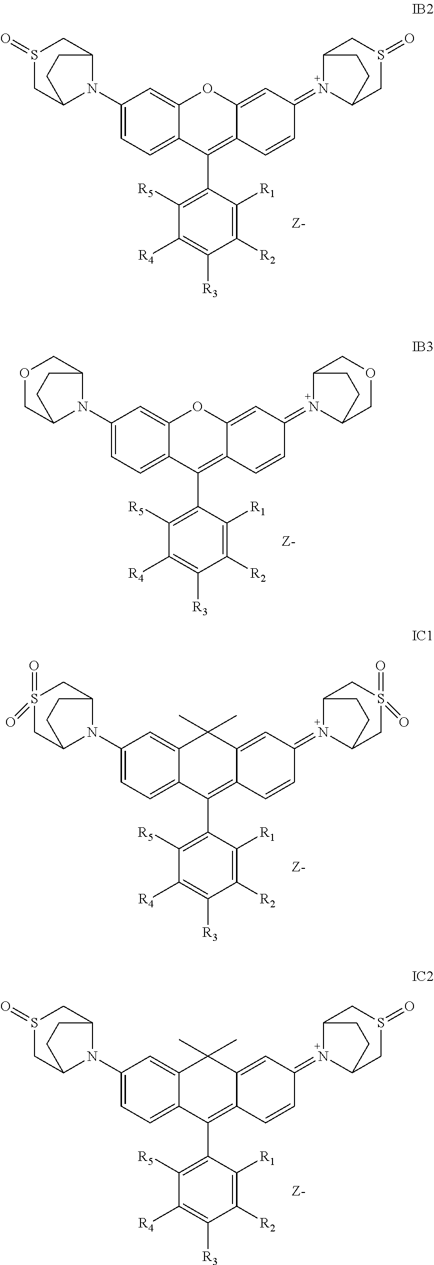

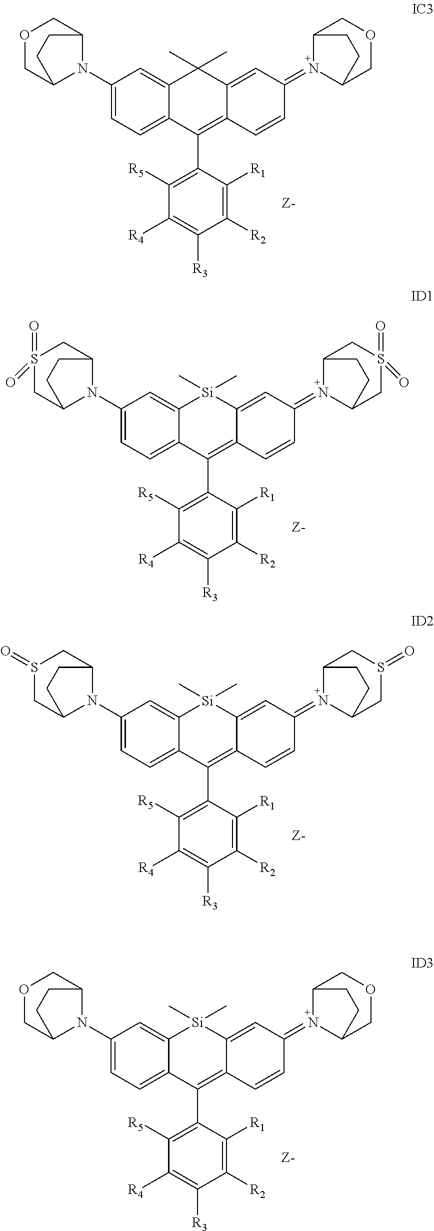

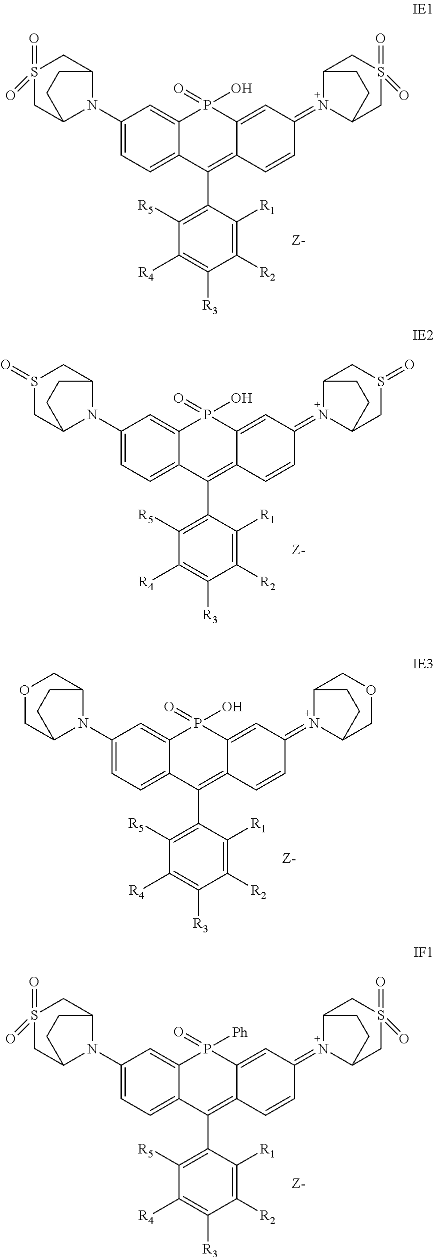

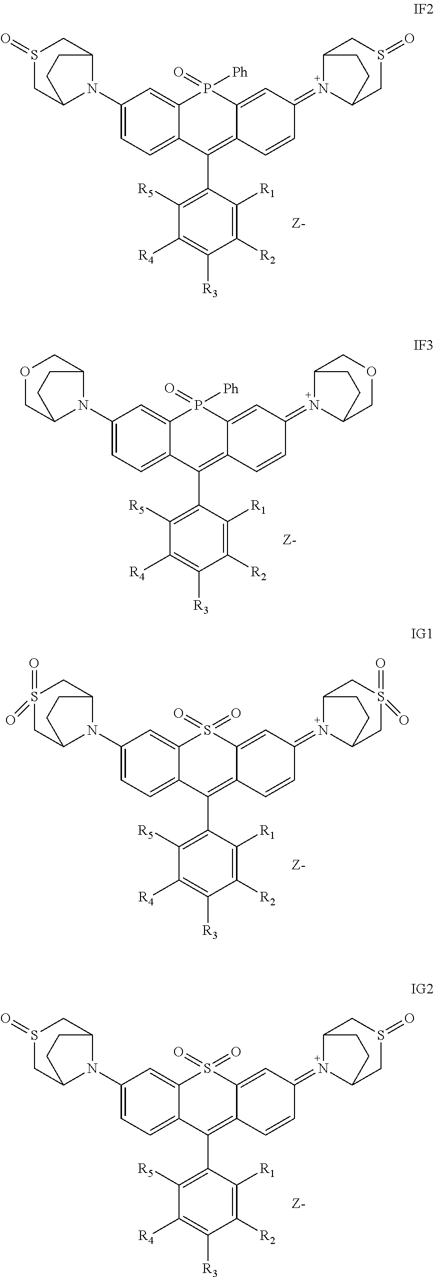

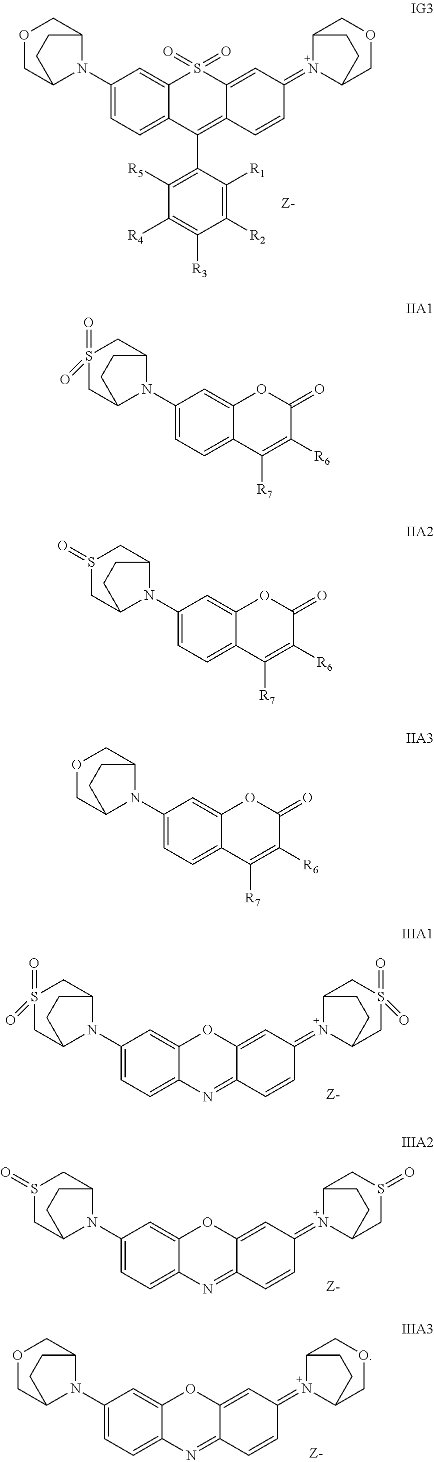

15. The aza[3.2.1]bridged ring substituted fluorescent dye according to claim 11, wherein the aza[3.2.1]bridged ring substituted fluorescent dye is selected from at least one of the following formulas A528, A532, A555, A586, A591, A620, A634, A640, A655, A720, A586-2Me, A586-2Me-5-COOH), A528-6-COOH), A532-6-COOH), A555-6-COOH), A586-6-COOH), A591-6-COOH), A620-6-COOH), A634-5-COOH), A640-6-COOH), A655-6-COOH), A528-HTL, A532-HTL, A555-HTL, A586-HTL, A620-HTL, A655-HTL, A555-STL, A528-NHS, A620-STL, A655-STL, A634-NHS, A586-NHS, A591-HTL, A640-HTL, A555-TMP3, A620-TMP3, A655-TMP3, A655-4-Hoechst, A591-TMP3, A640-TMP3, A591-STL, A640-STL, A528-NCN-HTL, A555-NCN-HTL, A620-NCN-HTL, B350, B356, C622, and C654:

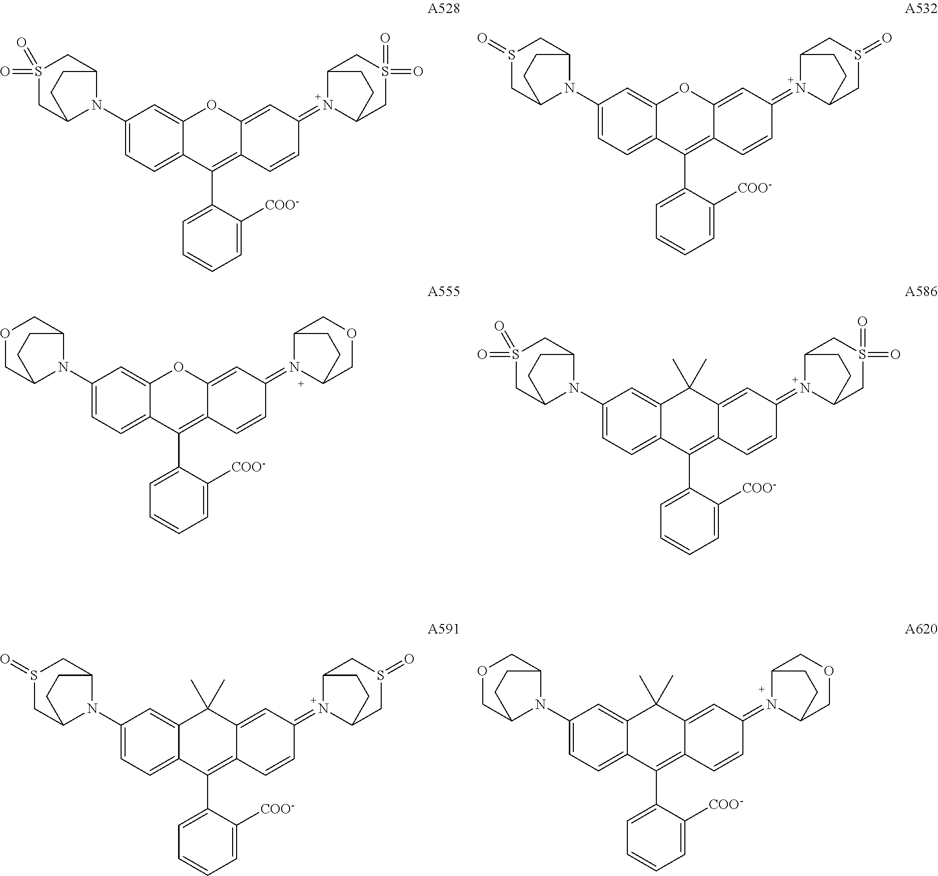

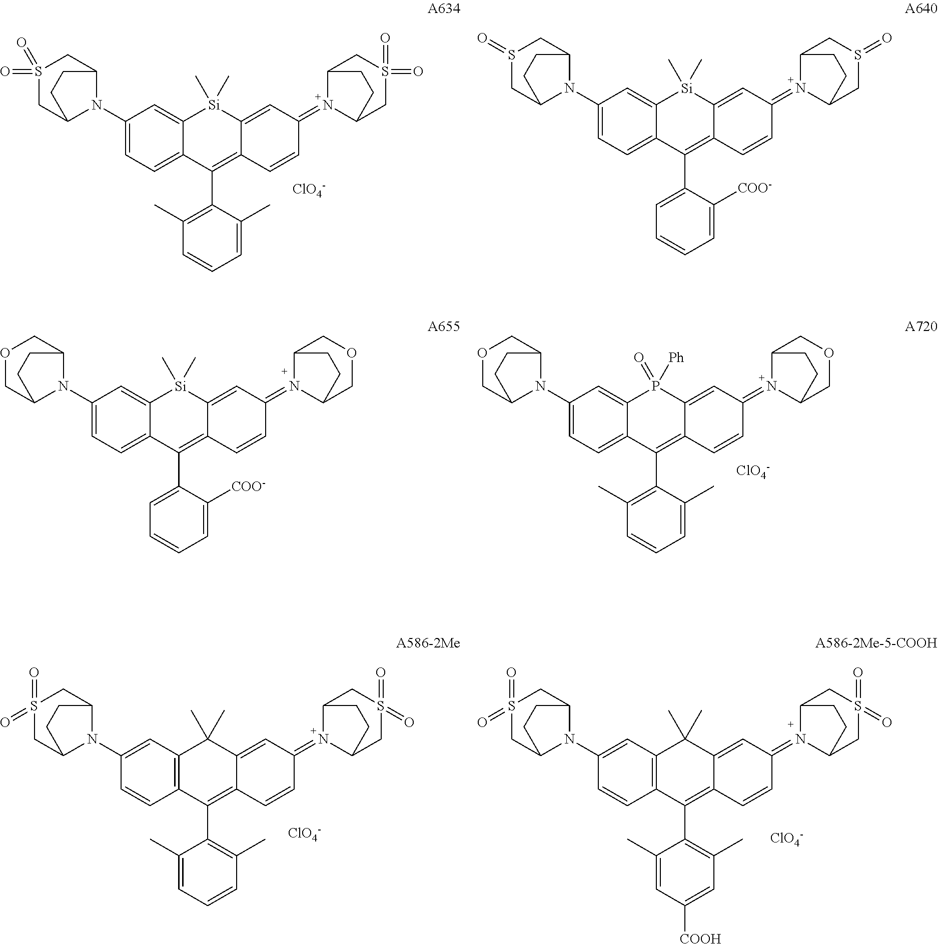

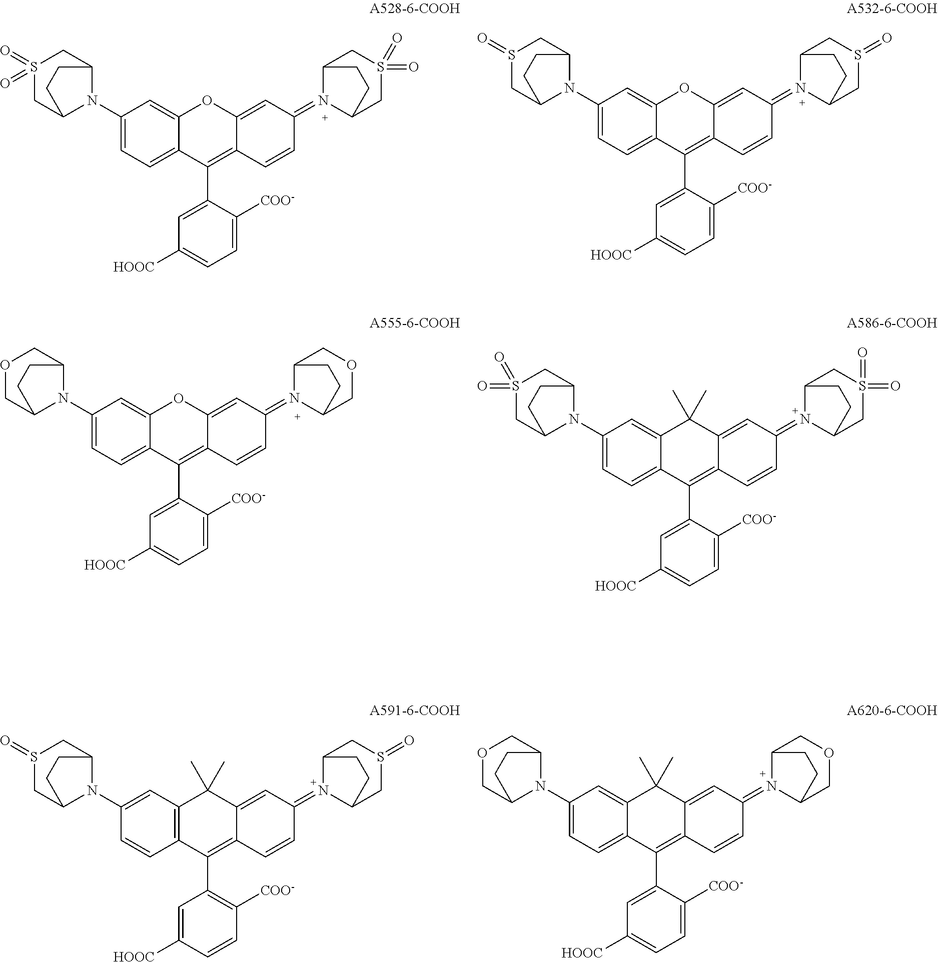

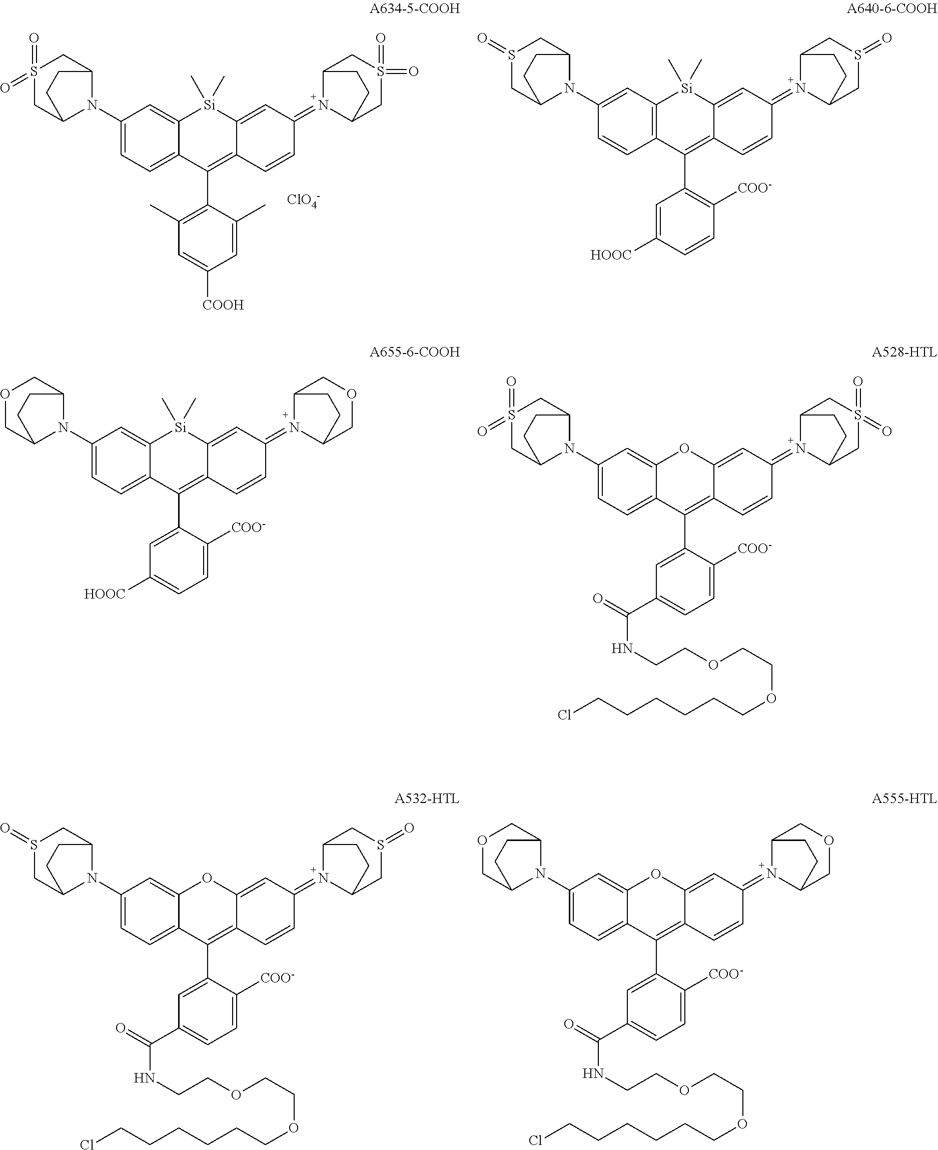

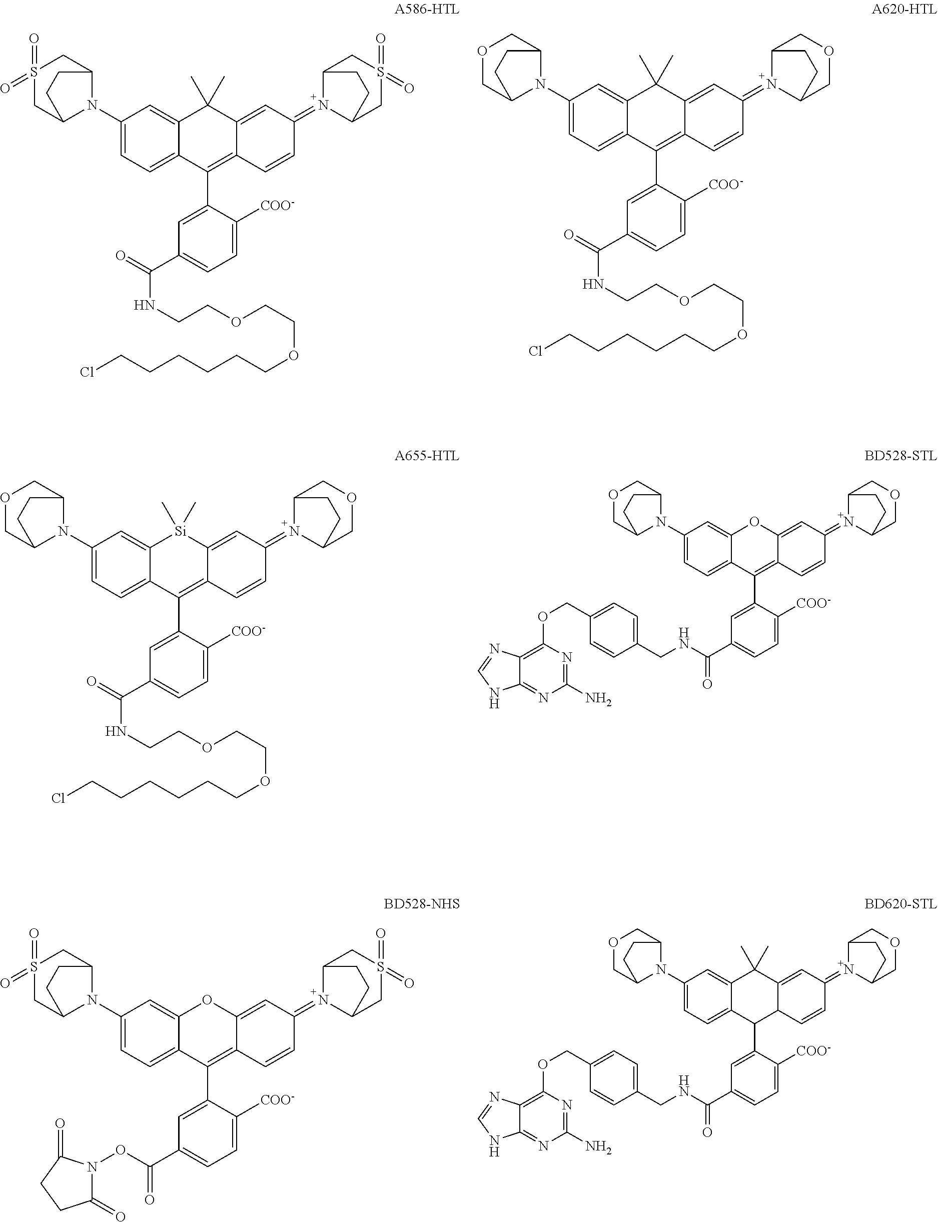

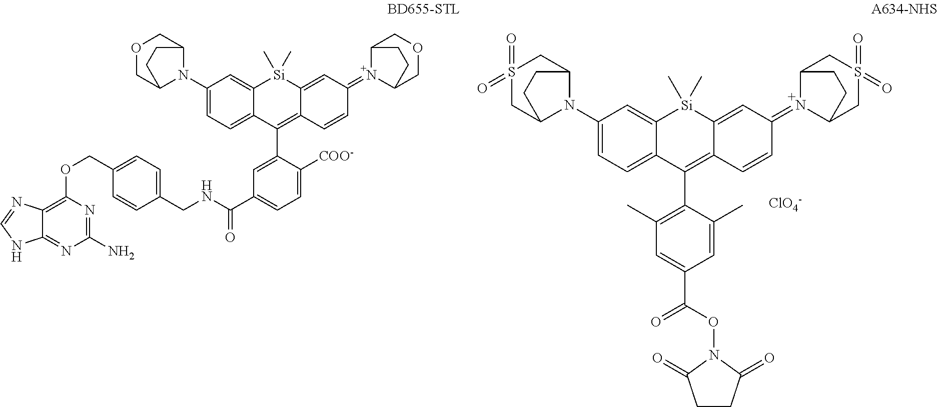

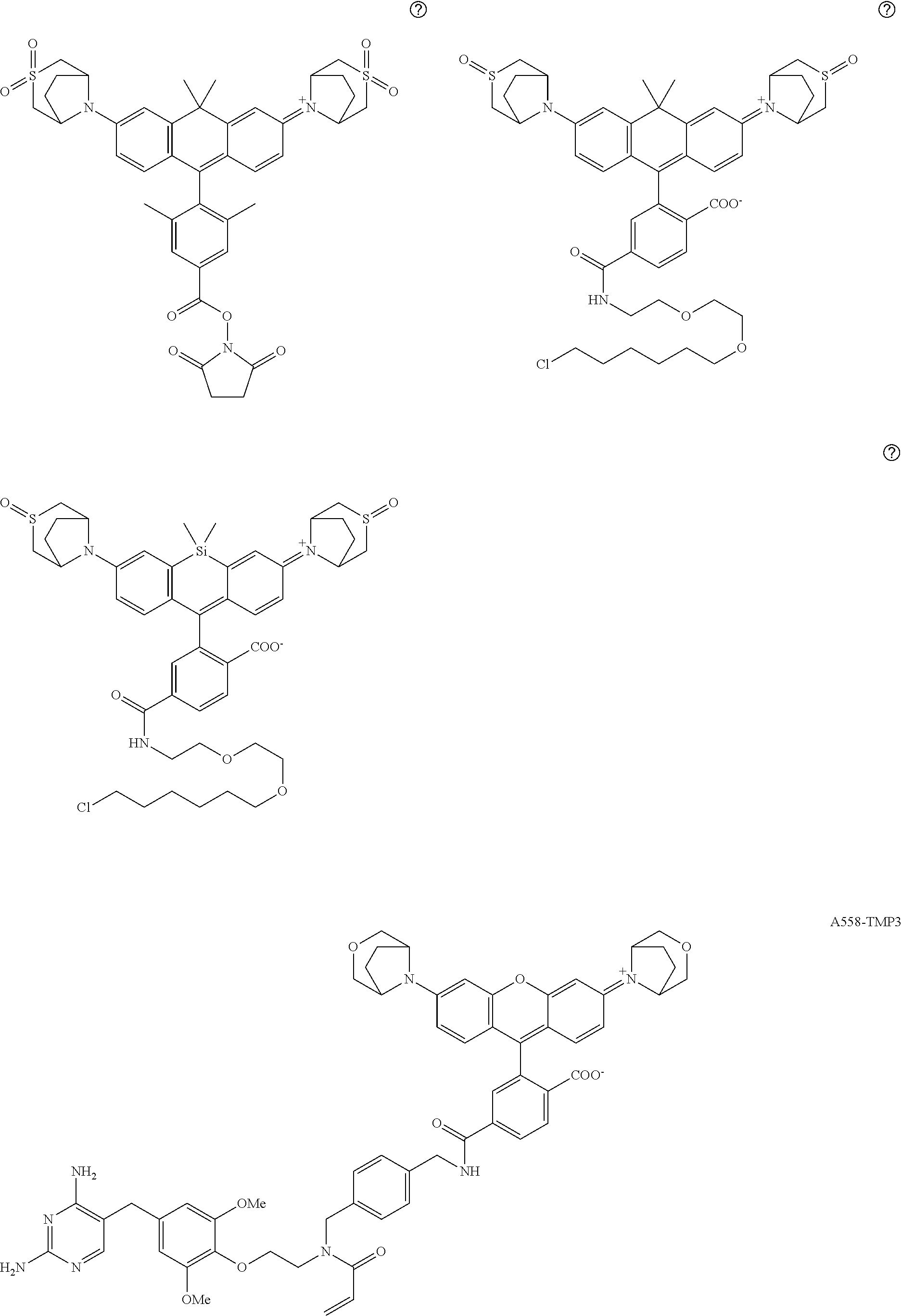

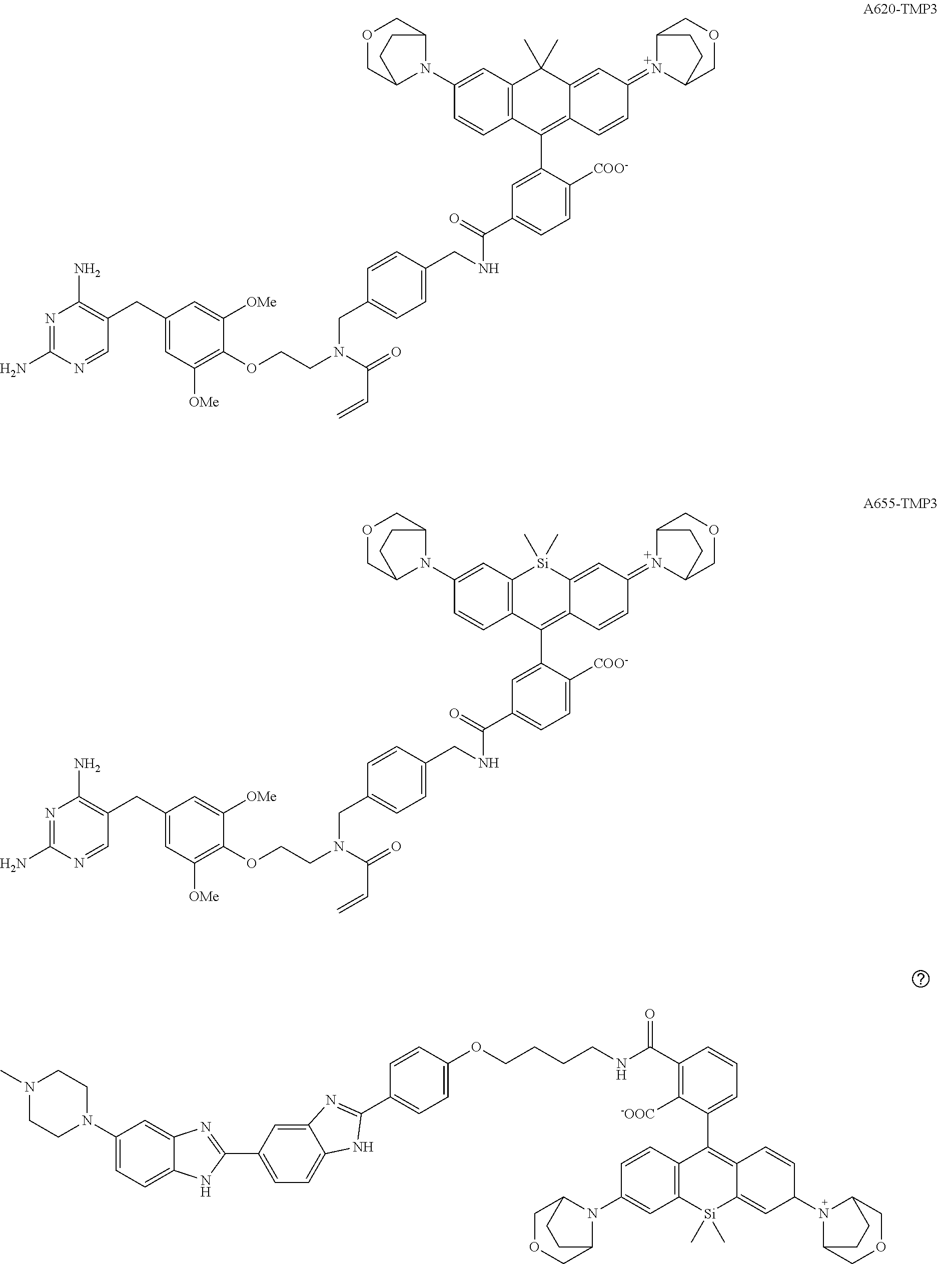

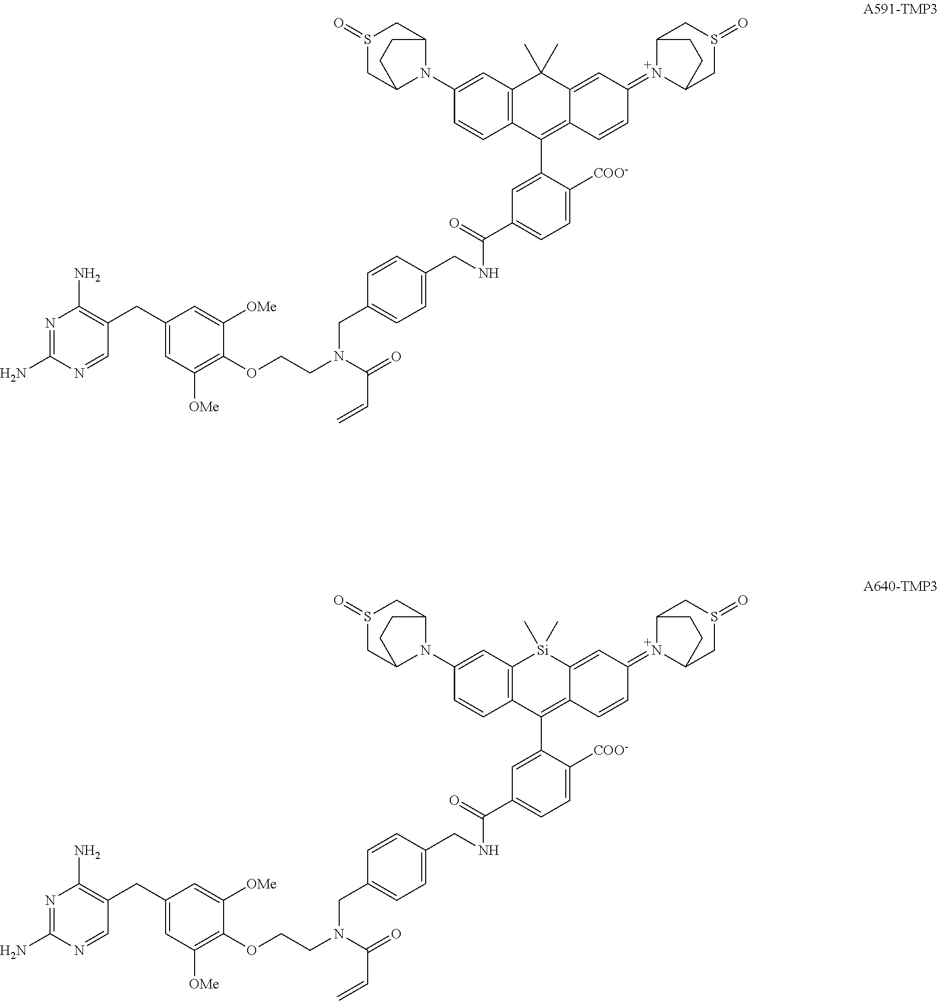

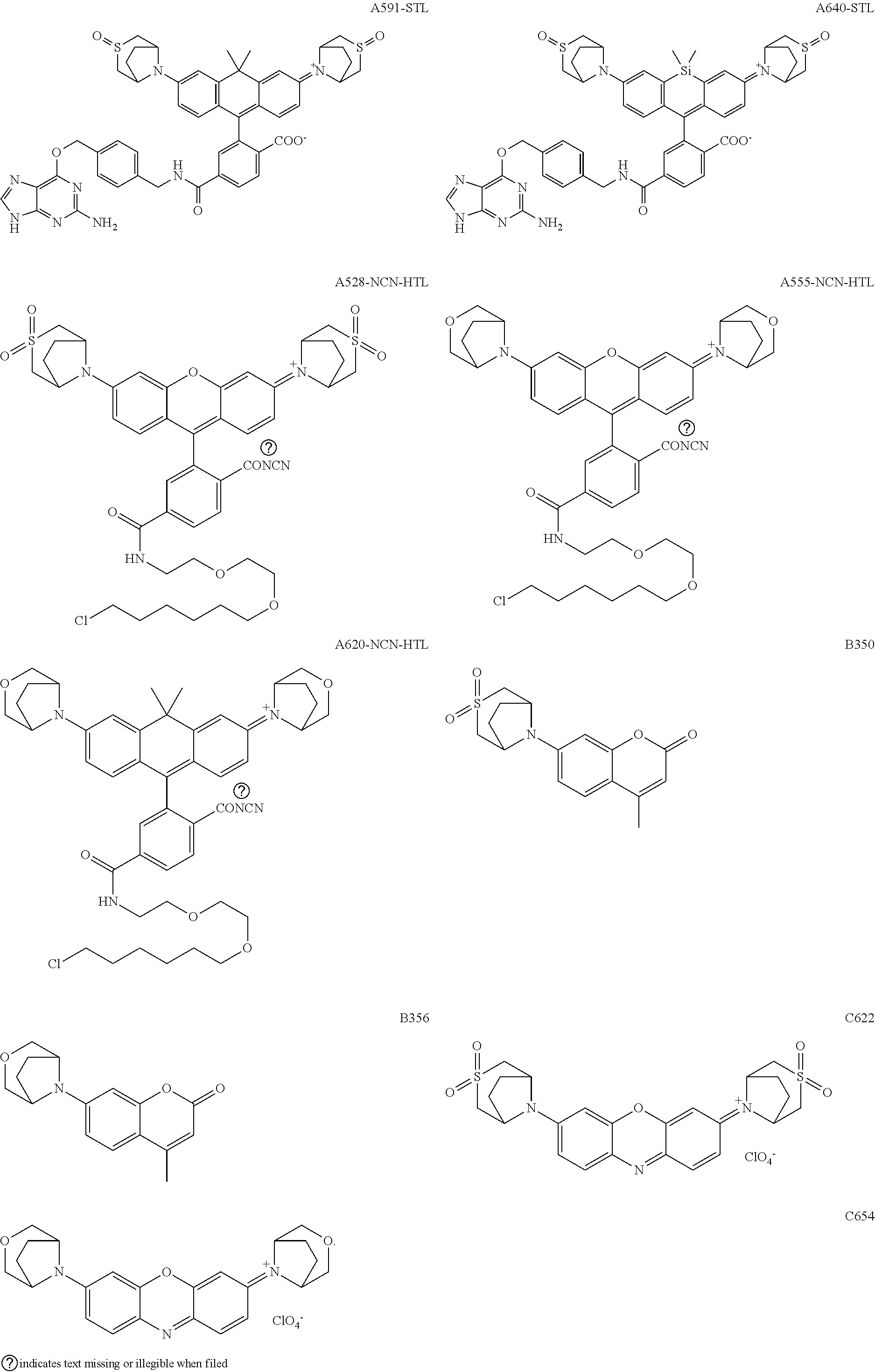

16. A method for preparing an aza[3.2.1]bridged ring substituted fluorescent dye, wherein the preparation method comprises:
step S11, mixing a compound A, an aza[3.2.1]bridged ring compound and a first catalyst in a first reaction solvent to carry out a reaction so as to obtain a compound B; and
step S12, mixing the compound B with an HZ aqueous solution to carry out a reaction so as to obtain the aza[3.2.1]bridged ring substituted fluorescent dye shown in Formula (I);
the compound A has a structure shown in Formula (V) below, and the aza[3.2.1]bridged ring compound has a structure shown in Formula (VI) below: 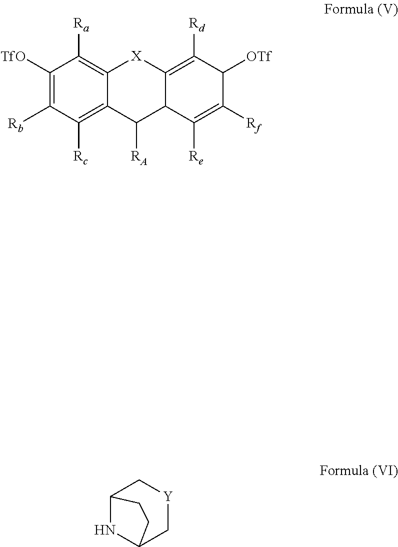
wherein R_{A}, Rₐ, R_{b}, R_{c}, R_{d}, Rₑ, R_{f}, X and Y independently have the same meaning as defined in claim 11, and Z⁻ in the HZ aqueous solution has the same meaning as defined in claim 11.

17. A method for preparing an aza[3.2.1]bridged ring substituted fluorescent dye, wherein the preparation method comprises:
mixing a compound C, an aza[3.2.1]bridged ring compound and a second catalyst in a second reaction solvent to carry out a reaction so as to obtain the aza[3.2.1]bridged ring substituted fluorescent dye shown in Formula (II);
the compound C has a structure shown in Formula (VII) below: 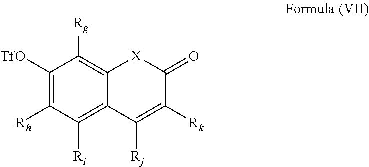
wherein R_{g}, Rₕ, Rᵢ, Rⱼ, Rₖ and X independently have the same meaning as defined in claim 11;
preferably, the second catalyst has the same meaning as the first catalyst;
and preferably, the second reaction solvent has the same meaning as the first reaction solvent.

18. A method for preparing an aza[3.2.1]bridged ring substituted fluorescent dye, wherein the preparation method comprises:
step S31, mixing a compound D, an aza[3.2.1]bridged ring compound and a third catalyst in a third reaction solvent to carry out a reaction so as to obtain a compound E;
step S32, mixing the compound E with an oxidizing agent in a fourth reaction solvent to carry out a reaction so as to obtain a compound F; and
step S33, mixing the compound F with an HZ aqueous solution to carry out a reaction so as to obtain the aza[3.2.1]bridged ring substituted fluorescent dye shown in Formula (III);
the compound D has a structure shown in Formula (VIII) below: 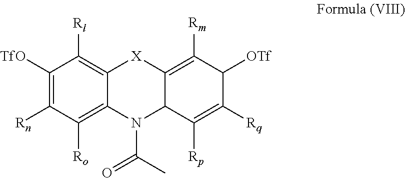
wherein Rₗ, Rₘ, Rₙ, Rₒ, Rₚ, R_{q}, X and Y independently have the same meaning as defined in claim 11, and Z⁻ in the HZ aqueous solution has the same meaning as defined in claim 11;
preferably, the third catalyst has the same meaning as the first catalyst;
preferably, the third reaction solvent has the same meaning as the first reaction solvent.

19. A use of the aza[3.2.1]bridged ring substituted fluorescent dye according to claim 11 in fields of fluorescence analysis, fluorescence detection and fluorescence imaging as a fluorescent marker.

20. The use of the aza[3.2.1]bridged ring substituted fluorescent dye according to claim 19, wherein the aza[3.2.1]bridged ring substituted fluorescent dye is used in fluorescence analysis, fluorescence detection, biomolecular labeling, immunofluorescence imaging, live-cell imaging, tissue imaging, in vivo imaging, single-molecule imaging and super-resolution fluorescence imaging as a fluorescent marker.

21. The method for preparing an aza[3.2.1]bridged ring substituted fluorescent dye according to claim 16, wherein the first catalyst comprises a palladium catalyst, a palladium catalyst ligand and an alkaline catalyst.

22. The method for preparing an aza[3.2.1]bridged ring substituted fluorescent dye according to claim 21, wherein the palladium catalyst comprises at least one of tetrakis(triphenylphosphine)palladium, palladium acetate, tris(dibenzylideneacetone)dipalladium, [(diphenylphosphino)ferrocene]dichloropalladium, dichlorobis(triphenylphosphine)palladium and palladium chloride, preferably at least one of tetrakis(triphenylphosphine)palladium, palladium acetate or tris(dibenzylideneacetone)dipalladium.

23. The method for preparing an aza[3.2.1]bridged ring substituted fluorescent dye according to claim 21, wherein the palladium catalyst ligand comprises at least one of bis(dibenzylideneacetone)palladium, 2,2′-bis(diphenylphosphino)-1,1′-binaphthyl, 2-dicyclohexylphosphino-2′,4′,6′-triisopropylbiphenyl and 2-dicyclohexylphosphino-2′,6′-dimethoxybiphenyl, preferably at least one of bis(dibenzylideneacetone)palladium and 2,2′-bis(diphenylphosphino)-1,1′-binaphthyl.

24. The method for preparing an aza[3.2.1]bridged ring substituted fluorescent dye according to claim 21, wherein the alkaline catalyst comprises at least one of cesium carbonate, sodium tert-butoxide, potassium carbonate, sodium carbonate, sodium methoxide, sodium ethoxide and potassium tert-butoxide, preferably at least one of cesium carbonate, sodium tert-butoxide and potassium tert-butoxide.

25. The method for preparing an aza[3.2.1]bridged ring substituted fluorescent dye according to claim 21, wherein the first reaction solvent comprises at least one of n-hexane, tetrahydrofuran, 1,4-dioxane, ethyl ether and toluene.

26. The method for preparing an aza[3.2.1]bridged ring substituted fluorescent dye according to claim 18, wherein, in step S32, the fourth reaction solvent is a mixed solvent of dichloromethane and water, and the volume ratio of the dichloromethane to the water is (10-30):1.

26. The method for preparing an aza[3.2.1]bridged ring substituted fluorescent dye according to claim 18, wherein, in step S32, the oxidizing agent comprises at least one of 2,3-dichloro-5,6-dicyano-1,4-benzoquinone, potassium permanganate, hydrogen peroxide and m-chloroperoxybenzoic acid, preferably 2,3-dichloro-5,6-dicyano-1,4-benzoquinone.

28. The method for preparing an aza[3.2.1]bridged ring substituted fluorescent dye according to claim 18, wherein, the molar ratio of the oxidizing agent to the compound E is (1.0-2.0):1.

29. The use of the aza[3.2.1]bridged ring substituted fluorescent dye according to claim 19, wherein the Rₐ, the R_{b}, the R_{c}, the Rₐ, the Rₑ, the R_{f}, the R_{g}, the Rₕ, the Rᵢ, the Rⱼ, the Rₖ, the Rₗ, the Rₘ, the Rₙ, the Rₒ, the Rₚ and the R_{q} are independently selected from hydrogen, halogen, carboxyl, substituted or unsubstituted C₁-C₁₀ alkyl, substituted or unsubstituted C₃-C₁₀ cycloalkyl, substituted or unsubstituted C₄-C₁₀ cycloalkylalkyl, C₄-C₁₀ alkylcycloalkyl, substituted or unsubstituted C₁-C₁₀ alkoxyl, substituted or unsubstituted C₁-C₁₀ ester group, and substituted or unsubstituted C₁-C₁₀ acylamino;
and/or, the R_{A} is selected from substituted or unsubstituted C₆-C₃₀ aryl, substituted or unsubstituted C₆-C₃₀ aralkyl, and substituted or unsubstituted C₅-C₃₀ heteroaryl;
and/or, the X is selected from oxygen, sulfur, substituted or unsubstituted C₁-C₁₀ alkyl, 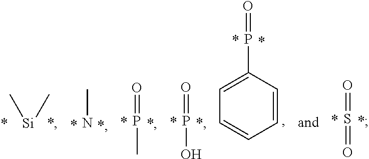
and/or, the Y is selected from oxygen, sulfur, nitrogen, substituted or unsubstituted C₁-C₁₀ alkyl, substituted or unsubstituted C₁-C₁₀ alkoxyl, substituted or unsubstituted C₁-C₁₀ acyl, substituted or unsubstituted C₁-C₁₀ ester group, 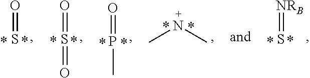
wherein the R_{B} is selected from substituted or unsubstituted C₁-C₂₀ alkyl, substituted or unsubstituted C₁-C₂₀ alkoxyl, substituted or unsubstituted C₁-C₂₀ acyl, and substituted or unsubstituted C₁-C₂₀ ester group;
and/or, the Z⁻ is selected from F⁻, Cl⁻, Br⁻, I⁻, OAc⁻, HSO₄⁻, H₂PO₄⁻, ClO₄⁻, CF₃COO⁻, CF₃SO₃⁻, CH₃SO₃⁻, NO₃⁻, C₅H₇O₅COO⁻, and PF₆⁻;
and/or, the m is an integer ranged from 1 to 3.

30. The use of the aza[3.2.1]bridged ring substituted fluorescent dye according to claim 19, wherein, the aza[3.2.1]bridged ring substituted fluorescent dye shown in Formula (I) has a structure shown in Formula (IV) below: 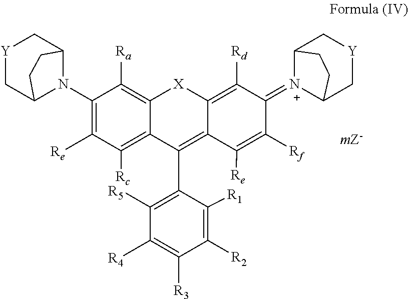
R₁ and R₅ are independently selected from hydrogen, carboxyl, halogen, substituted or unsubstituted C₁-C₆ alkyl, substituted or unsubstituted C₁-C₆ alkoxyl, nitro, amino, hydroxyl, 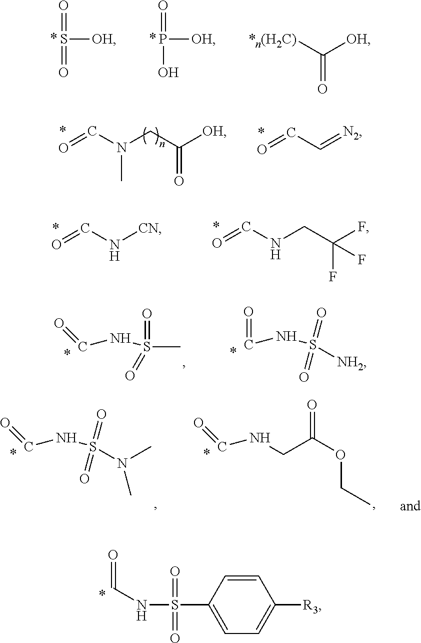
wherein R_{D} is selected from hydrogen, halogen, cyano, substituted or unsubstituted C₁-C₆ alkyl, and substituted or unsubstituted C₁-C₆ alkoxyl, and n is an integer ranged from 1 to 6; and R₂, R₃ and R₄ are independently selected from hydrogen, halogen, carboxyl, substituted or unsubstituted C₁-C₁₀ alkyl, substituted or unsubstituted C₃-C₁₀ cycloalkyl, substituted or unsubstituted C₄-C₁₀ cycloalkylalkyl, C₄-C₁₀ alkylcycloalkyl, substituted or unsubstituted C₁-C₁₀ alkoxyl, substituted or unsubstituted C₁-C₁₀ ester group, [chemical structure]
